(19) United States
(12) Patent Application Publication
(10) Pub. No.: US 2006/0247272 A1
(43) Pub. Date: Nov. 2, 2006

(54) 4-Amino Substituted-2-Substituted-1,2,3,4-tetrahydroquinoline Compounds

(75) Inventors: Roger B. Ruggeri, Waterford, CT (US); George Magnus-Aryitey, Ledyard, CT (US); Ravi M. Shanker, Groton, CT (US); Douglas A. Lorenz, Bend, OR (US); Cheryl D. Garr, Redmond, OR (US)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 11/424,601

(22) Filed: Jun. 16, 2006

Related U.S. Application Data
(62) Division of application No. 11/187,854, filed on Jul. 25, 2005.
(60) Provisional application No. 60/658,704, filed on Mar. 3, 2005.
(60) Provisional application No. 60/612,860, filed on Sep. 23, 2004.

(51) Int. Cl. A61K 31/4706; C07D 215/38

(52) U.S. Cl. 514/313; 546/159

(57) ABSTRACT

4-Amino substituted-2-substituted-1,2,3,4-tetrahydroquinoline compounds, pharmaceutical compositions containing such compounds and the use of such compounds to elevate certain plasma lipid levels, including high density lipoprotein-cholesterol and to lower certain other plasma lipid levels, such as LDL-cholesterol and triglycerides and accordingly to treat diseases which are exacerbated by low levels of HDL cholesterol and/or high levels of LDL-cholesterol and triglycerides, such as atherosclerosis and cardiovascular diseases in some mammals, including humans.

### BACKGROUND OF INVENTION

This invention relates to 4-amino substituted-2-substituted-1,2,3,4-tetrahydroquinoline compounds, pharmaceutical compositions containing such compounds and the use of such compounds to elevate certain plasma lipid levels, including high density lipoprotein (HDL)-cholesterol and to lower certain other plasma lipid levels, such as low density lipoprotein (LDL)-cholesterol and triglycerides and accordingly to treat diseases which are affected by low levels of HDL cholesterol and for high levels of LDL-cholesterol and triglycerides, such as atherosclerosis and cardiovascular diseases in certain mammals (i.e., those which have CETP in their plasma), including humans.

Atherosclerosis and its associated coronary artery disease (CAD) is the leading cause of mortality in the industrialized world. Despite attempts to modify secondary risk factors (smoking, obesity, lack of exercise) and treatment of dyslipidemia with dietary modification and drug therapy, coronary heart disease (CHD) remains the most common cause of death in the U.S., where cardiovascular disease accounts for 44% of all deaths, with 53% of these associated with atherosclerotic coronary heart disease.

Risk for development of this condition has been shown to be strongly correlated with certain plasma lipid levels. While elevated LDL-C may be the most recognized form of dyslipidemia, it is by no means the only significant lipid associated contributor to CHD. Low HDL-C is also a known risk factor for CHD (Gordon, D. J., et al., “High-density Lipoprotein Cholesterol and Cardiovascular Disease”, Circulation, (1989), 79: 8-15).

High LDL-cholesterol and triglyceride levels are positively correlated, while high levels of HDL-cholesterol are negatively correlated with the risk for developing cardiovascular diseases. Thus, dyslipidemia is not a unitary risk profile for CHD but may be comprised of one or more lipid aberrations.

Among the many factors controlling plasma levels of these disease dependent principles, cholesteryl ester transfer protein (CETP) activity affects all three. The role of this 70,000 dalton plasma glycoprotein found in a number of animal species, including humans, is to transfer cholesteryl ester and triglyceride between lipoprotein particles, including high density lipoproteins (HDL), low density lipoproteins (LDL), very low density lipoproteins (LDL), and chylomicrons. The net result of CETP activity is a lowering of HDL cholesterol and an increase in LDL cholesterol. This effect on lipoprotein profile is believed to be pro-atherogenic, especially in subjects whose lipid profile constitutes an increased risk for CHD.

No wholly satisfactory HDL-elevating therapies are on the market today. Niacin can significantly increase HDL, but has serious toleration issues which reduce compliance. Fibrates and the HMG CoA reductase inhibitors raise HDL-C, but in some patients, the result is an increase of modest porportions (˜10-12%). As a result, there is an unmet medical need for an approved therapeutic agent that elevates plasma HDL levels, thereby reversing or slowing the progression of atherosclerosis.

CETP inhibitors, particularly those that have high binding activity, are generally hydrophobic and are difficult to isolate in a pharmaceutically acceptable crystalline form for manufacturing. In addition, some CETP inhibitors are known to have some amount of hypertensive activity. Specific examples of CETP inhibitors include [2R,4S]4-[(3,5-bis-trifluoromethyl-benzyl)-methoxycarbonyl-amino]-2-ethyl-6-trifluoromethyl-3,4-dihydro-2H-quinoline-1-carboxylic acid ethyl ester (torcetrapib), [2R,4S]4-[acetyl-(3,5-bis-trifluoromethyl-benzyl-amino]-2-ethyl-6-trifluoromethyl-3,4-dihydro-2H-quinoline-1-carboxylic acid isopropyl ester, [2R,4S]4-[(3,5-Bis-trifluoromethyl-benzyl)-methoxycarbonyl-amino-2-ethyl-6-trifluoromethyl-3,4-dihydro-2H-quinoline-1-carboxylic acid isopropyl ester, (2R)-3-[[3-(4-chloro-3-ethylphenoxy)phenyl][[3-(1,1,2,2-tetrafluoroethoxy)phenyl]methyl]amino]-1,1,1-trifluoro-2-propanol, S-[2-([[1-(2-ethylbutyl)cyclohexyl]carbonyl]amino)phenyl]2-methylpropanethioate, trans-4-[[[2-[[[3,5-bis(trifluoromethyl)phenyl]methyl](2-methyl-2H-tetrazol-5-yl)amino]methyl]-4-(trifluoromethyl)phenyl]ethylamino]methyl]-cyclohexaneacetic acid, trans-4-[[[2-[[[[3,5-bis(trifluoromethyl)phenyl]methyl](2-methyl-2H-tetrazol-5-yl)amino]methyl]-5-methyl-4-(trifluoromethyl)phenyl]ethylamino]methyl]-cyclohexaneacetic acid, the drugs disclosed in the commonly owned U.S. Patent Application Ser. No. 60/612,863 filed Sep. 23, 2004, the disclosure of which is incorporated herein by reference for all purposes, and the drugs disclosed in the following patents and published applications, the disclosures of all of which are incorporated herein by reference for all purposes: DE 19741400 A1; DE 19741399 A1; WO 9914215A1; WO 9914174; DE 19709125 A1; DE 19704244 A1; DE 19704243 A1; EP 818448 A1; WO 9804528A2; DE 19627431 A1; DE 19627430 A1; DE 19627419 A1; EP 796846 A1, DE 19832159; DE 818197; DE 19741051; WO 9941237 A1; WO 9914204 A1: WO 9836937 A1; JP 11049743; WO 200018721; WO 200018723; WO 200018724; WO 200017164; WO 200017165; WO 200017166; WO 2004020393; WO 2004085401; EP 992496; and EP 987251.

Thus, although there are a variety of anti-atherosclerosis therapies, there is a continuing need and a continuing search in this field of art for alternative therapies.

### SUMMARY OF THE INVENTION

This invention is directed to compounds of the Formula I 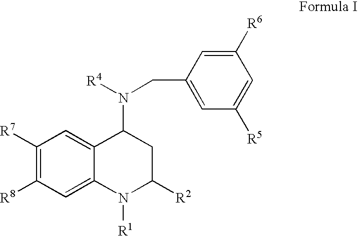
or a pharmaceutically acceptable salt of said compounds wherein;

R¹ is Y, W—O—Y or W—Y; wherein W is a carbonyl; Y for each occurrence is independently Z or (C₁-C₁₀)alkyl wherein one of the carbons may be replaced with S, O or N, and when Y is (C₁-C₁₀)alkyl then Y is optionally substituted with one to nine substitutents independently selected from: halo, hydroxy, oxo, amino, amido, carboxy, and Z; wherein Z is a partially saturated, fully saturated or fully unsaturated three to eight membered ring or bicyclic ring system optionally having one to four heteroatoms selected from O, S and N wherein Z is optionally substituted with one, two or three substitutents independently selected from halo, (C₁-C₆) alkyl, hydroxy, (C₁-C₆)alkoxy, amino, amido, cyano, oxo, carboxy, (C₁-C₆)alkyloxycarbonyl, mono-N-and di-N,N-(C₁-C₆)alkylamino wherein said (C₁-C₆)alkyl substituent is optionally substituted with one, two or three substituents independently selected from halo, hydroxy, (C₁-C₆)alkoxy, cyano, oxo, amino, amido, carboxy, mono-N- and di-N,N-(C₁-C₆)alkylamino, and (C₁-C₆)alkyloxycarbonyl, said (C₁-C₆)alkyl or (C₁-C₆)alkoxy substituent is also optionally substituted with from one to nine fluorines;

R² is (C₁-C₄)alkyl or (C₁-C₆)cycloalkyl;

R⁴ is V⁰, —COO(C₁-C₄)alkyl, cyano, —CHO, —CONH₂, or —CO(C₃-C₄)alkyl; wherein V⁰ is tetrazolyl, triazolyl, imidazolyl, pyrazolyl, oxadiazolyl, isoxazolyl, furanyl, thiadiazolyl, isothiazolyl, thiophenyl, pyrimidinyl, or pyridinyl: wherein V⁰ is optionally substituted with (R⁰)ₙ wherein n is 1, 2, 3 or 4 and each R⁰ is independently halo, (C₁-C₆)alkyl, hydroxy, (C₁-C₆)alkoxy, amino, amido, cyano, oxo, carboxamoyl, carboxy, or (C₁-C₆)alkyloxycarbonyl, wherein said (C₁-C₆)alkyl or (C₁-C₆)alkoxy substituent is optionally independently substituted with one or two oxo, one or two hydroxy, or one to nine halo; and

R⁵, R⁶, R⁷, and R⁸ are independently hydrogen, cyano, halo, (C₁-C₄)alkoxy or (C₁-C₄)alkyl wherein said (C₁-C₄)alkyl and (C₁-C₄)alkoxy are optionally substituted independently with from one to seven halo; with the proviso that when R⁴ is other than V⁰ then R¹ is not (C₁-C₆)alkyl and R¹ has an amido substituent or carboxy substituent.

The present invention is further directed to compounds of the Formula II 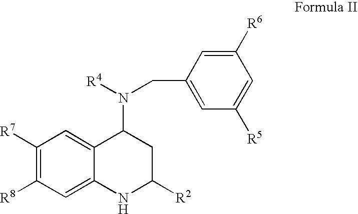
or a pharmaceutically acceptable salt of said compound, wherein

R² is (C₁-C₄) or (C₁-C₆)cycloalkyl;

R⁴ is tetrazolyl optionally substituted with (R⁰)ₙ wherein n is 1, 2, 3 or 4 and each R⁰ is independently halo, (C₁-C₅)alkyl, hydroxy, (C₁-C₆)alkoxy, amino, amido, cyano, oxo, carboxamoyl, carboxy, or (C₁-C₆)alkyloxycarbonyl, wherein said (C₁-C₆)alkyl or (C₁-C₆)alkoxy substituent is optionally independently substituted with one or two oxo, one or two hydroxy, or one to nine halo; and

R⁵, R⁶, R⁷, and R⁵ are independently hydrogen, cyano, halo, (C₁-C₄)alkoxy or (C₁-C₄)alkyl wherein said (C₁-C₄)alkyl and (C₁-C₄)alkoxy are optionally substituted independently with from one to seven halo.

The present invention is further directed to 2-(4-{4-[(3,5-Bis-trifluoromethyl-benzyl)-(2-methyl-2H-tetrazol-5-yl)-amino]-2-ethyl-6-trifluoromethyl-3,4-dihydro-2H-quinoline-1 carbonyl}-cyclohexyl)-acetamide or a pharmaceutically acceptable salt of said compound; further to (2R,4S)-2-(4-{4-[(3,5-Bis-trifluoromethyl-benzyl)-(2-methyl-2H-tetrazol-5-yl)-amino]-2-ethyl-6-trifluoromethyl-3,4-dihydro-2H-quinoline-1-carbonyl}-cyclohexyl)-acetamide; and further to Trans-(2R,4S)-2-(4-{4-[(3,5-Bis-trifluoromethyl-benzyl)-(2-methyl-2H-tetrazol-5-yl)-amino]-2-ethyl-6-trifluoromethyl-3,4-dihydro-2H-quinoline-1-carbonyl}-cyclohexyl)-acetamide and Cis-(2R,4S)-2-{4-(4-[(3,5-Bis-trifluoromethyl-benzyl)-(2-methyl-2H-tetrazol-5-yl)-amino]-2-ethyl-6-trifluoromethyl-3,4-dihydro-2H-quinoline-1-carbonyl}-cyclohexyl)-acetamide, and pharmaceutically acceptable salts of said compounds.

Moreover, the present invention is directed to compounds of Formulas III and IV:

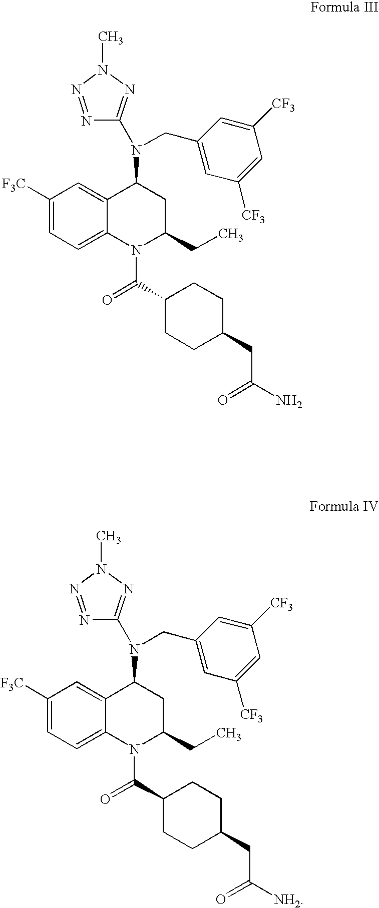

In addition, the present invention provides methods for treating atherosclerosis, coronary artery disease, coronary heart disease, coronary vascular disease, peripheral vascular disease, dyslipidemia, hyperbetalipoproteinemia, hypoalphalipoproteinemia, hypercholesterolemia, hypertriglyceridemia, familial-hypercholesterolemia or myocardial infarction in a mammal by administering to a mammal in need of such treatment an atherosclerosis, coronary artery disease, coronary heart disease, coronary vascular disease, peripheral vascular disease, dyslipidemia, hyperbetalipoproteinemia, hypoalphalipoproteinemia, hypercholesterolemia, hypertriglyceridemia, familial-hypercholesterolemia or myocardial infarction treating amount of a compound of the present invention, or a pharmaceutically acceptable form of said compound.

In addition, the present invention provides pharmaceutical compositions which comprise a therapeutically effective amount of a compound of the present invention, or a pharmaceutically acceptable form of said compound and a pharmaceutically acceptable vehicle, diluent or carrier.

In addition, the present invention provides pharmaceutical compositions for the treatment of atherosclerosis, coronary artery disease, coronary heart disease, coronary vascular disease, peripheral vascular disease, dyslipidemia, hyperbetalipoproteinemia, hypoalphalipoproteinemia, hypercholesterolemia, hypertriglyceridemia, familial-hypercholesterolemia or myocardial infarction in a mammal which comprise a therapeutically effective amount of a compound of the present invention, or a pharmaceutically acceptable form of said compound and a pharmaceutically acceptable vehicle, diluent or carrier.

Moreover, the present invention provides pharmaceutical combination compositions comprising a therapeutically effective amount of a composition comprising

a first compound, said first compound being a compound of the present invention, or a pharmaceutically acceptable form of said compound;

at least one second compound, said second compound being an HMG CoA reductase inhibitor, an MTP/Apo B secretion inhibitor, a PPAR modulator, an antihypertensive, a bile acid reuptake inhibitor, a cholesterol absorption inhibitor, a cholesterol synthesis inhibitor, a fibrate, niacin, slow-release niacin, a combination of niacin and lovastatin, a combination of niacin and simvastatin, a combination of niacin and atorvastatin, a combination of amlodipine and atorvastatin, an ion-exchange resin, an antioxidant, an ACAT inhibitor or a bile acid sequestrant, or a pharmaceutically acceptable salt of said second compound (preferably an HMG-CoA reductase inhibitor, a PPAR modulator, niacin, fenofibrate, lovastatin, simvastatin, pravastatin, fluvastatin, atorvastatin, rivastatin, rosuvastatin or pitavastatin), and

a pharmaceutical vehicle, diluent or carrier. This composition may be used to treat the aforementioned diseases, including atherosclerosis.

Also, the present invention provides a kit for achieving a therapeutic effect in a mammal comprising packaged in association a first therapeutic agent comprising a therapeutically effective amount of a compound of the present invention, a prodrug thereof, or a pharmaceutically acceptable salt of said compound or of said prodrug and a pharmaceutically acceptable carrier, at least one second therapeutic agent comprising a therapeutically effective amount of an HMG CoA reductase inhibitor, an MTP/Apo B secretion inhibitor, a PPAR modulator, an antihypertensive, a bile acid reuptake inhibitor, a cholesterol absorption inhibitor, a cholesterol synthesis inhibitor, a fibrate, niacin, slow-release niacin a combination of niacin and lovastatin, a combination of niacin and simvastatin, a combination of niacin and atorvastatin, a combination of amlodipine and atorvastatin, an ion-exchange resin, an antioxidant, an ACAT inhibitor or a bile acid sequestrant, or a pharmaceutically acceptable salt of said second therapeutic agent; and a pharmaceutically acceptable carrier and directions for administration of said first and second agents to achieve the therapeutic effect.

It is to be understood that both the foregoing general description and the following detailed description are exemplary and explanatory only and are not restrictive of the invention, as claimed.

### BRIEF DESCRIPTION OF THE DRAWINGS

FIG. 1 is a representative differential scanning calorimetry thermogram of trans-(2R,4S)-2-(4-{4-[(3,5-bis-trifluoromethyl-benzyl)-(2-methyl-2H-tetrazol-5-yl)-amino]-2-ethyl-6-trifluoro methyl-3,4-dihydro-2H-quinoline-1-carbonyl}-cyclohexyl)-acetamide, form A, (Scan Rate: 5° C. per minute; Vertical Axis: Heat Flow (mW); Horizontal Axis: Temperature (° C.)).

FIG. 2 is a representative powder X-ray diffraction pattern for trans-(2R,4S)-2-(4-{4-[(3,5-bis trifluoromethyl-benzyl)-(2-methyl-2H-tetrazol-5-yl)-amino]-2-ethyl-6-trifluoromethyl-3,4-dihydro-2H-quinoline-1-carbonyl}-cyclohexyl)-acetamide, form A, (Vertical Axis: Intensity (counts): Horizontal Axis: Two Theta (Degrees)).

### DETAILED DESCRIPTION OF THE INVENTION

The present invention may be understood more readily by reference to the following detailed description of exemplary embodiments of the invention and the examples included therein.

Before the present compounds, compositions and methods are disclosed and described, it is to be understood that this invention is not limited to specific synthetic methods of making that may of course vary. It is also to be understood that the terminology used herein is for the purpose of describing particular embodiments only and is not intended to be limiting.

The present invention also relates to the pharmaceutically acceptable acid addition salts of compounds of the present invention. The acids which are used to prepare the pharmaceutically acceptable acid addition salts of the aforementioned base compounds of this invention are those which form non-toxic acid addition salts, (i.e., salts containing pharmacologically acceptable anions, such as the hydrochloride, hydrobromide, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate (i.e., 1,1′-methylene-bis-(2-hydroxy-3-naphthoate)) salts.

The invention also relates to base addition salts of the compounds of the present invention. The chemical bases that may be used as reagents to prepare pharmaceutically acceptable base salts of those compounds of the present invention that are acidic in nature are those that form non-toxic base salts with such compounds. Such non-toxic base salts include, but are not limited to those derived from such pharmacologically acceptable cations such as alkali metal cations (e.g., potassium and sodium) and alkaline earth metal cations (e.g., calcium and magnesium), ammonium or water-soluble amine addition salts such as N-methylglucamine-(meglumine), and the lower alkanolammonium and other base salts of pharmaceutically acceptable organic amines.

The chemist of ordinary skill will recognize that certain compounds of this invention will contain one or more atoms which may be in a particular stereochemical or geometric configuration, giving rise to stereoisomers and configurational isomers. All such isomers and mixtures thereof are included in this invention. Hydrates and solvates of the compounds of this invention are also included.

Where the compounds of the present invention possess two or more stereogenic centers and the absolute or relative stereochemistry is given in the name, the designations R and S refer respectively to each stereogenic center in ascending numerical order (1, 2, 3, etc.) according to the conventional IUPAC number schemes for each molecule. Where the compounds of the present invention possess one or more stereogenic centers and no stereochemistry is given in the name or structure, it is understood that the name or structure is intended to encompass all forms of the compound, including the racemic form.

The compounds of this invention may contain olefin-like double bonds. When such bonds are present, the compounds of the invention exist as cis and trans configurations and as mixtures thereof. The term “cis” refers to the orientation of two substituents with reference to each other and the plane of the ring (either both “up” or both “down”). Analogously, the term “trans” refers to the orientation of two substituents with reference to each other and the plane of the ring (the substituents being on opposite sides of the ring).

Alpha and Beta refer to the orientation of a substituent with reference to the plane of the ring. Beta is above the plane of the ring and Alpha is below the plane of the ring.

This invention also includes isotopically-labeled compounds, which are identical to those described by formulas I and II, except for the fact that one or more atoms are replaced by one or more atoms having specific atomic mass or mass numbers. Examples of isotopes that can be incorporated into compounds of the invention include isotopes of hydrogen, carbon, nitrogen, oxygen, sulfur, fluorine, and chlorine such as ²H, ³H, ¹³C, ¹⁴C, ¹⁵N, ¹⁸O, ¹⁷O, ¹⁸F, and ³⁵Cl respectively. Compounds of the present invention, prodrugs thereof, and pharmaceutically acceptable salts of the compounds or of the prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated (i.e., ³H), and carbon-14 (i.e., ¹⁴C), isotopes are particularly preferred for their ease of preparation and detectability. Further, substitution with heavier isotopes such as deuterium (i.e., ²H), can afford certain therapeutic advantages resulting from greater metabolic stability, for example increased in vivo half-life or reduced dosage requirements and, hence, may be preferred in some circumstances. Isotopically labeled compounds of this invention and prodrugs thereof can generally be prepared by carrying out the procedures disclosed in the schemes and/or in the Examples below, by substituting a readily available isotopically labeled reagent for a non-isotopically labeled reagent.

In this specification and in the claims that follow, reference will be made to a number of terms that shall be defined to have the following meanings.

As used herein, the term mammals is meant to refer to all mammals which contain CETP in their plasma, for example, rabbits and primates such as monkeys and humans, including males and females. Certain other mammals e.g., dogs, cats, cattle, goats, sheep and horses do not contain CETP in their plasma and so are not included herein.

The term “treating”, “treat” or “treatment” as used herein includes preventative (e.g., prophylactic) and palliative treatment.

By “pharmaceutically acceptable” is meant the carrier, diluent, excipients, and/or salt must be compatible with the other ingredients of the formulation, and not deleterious to the recipient thereof.

“Compounds” when used herein includes any pharmaceutically acceptable derivative or variation, including conformational isomers (e.g., cis and trans isomers) and all optical isomers (e.g., enantiomers and diastereomers), racemic, diastereomeric and other mixtures of such isomers, as well as solvates, hydrates, isomorphs, polymorphs, tautomers, esters, salt forms, and prodrugs. By “tautomers” is meant chemical compounds that may exist in two or more forms of different structure (isomers) in equilibrium, the forms differing, usually, in the position of a hydrogen atom. Various types of tautomerism can occur, including keto-enol, ring-chain and ring-ring tautomerism. The expression “prodrug” refers to compounds that are drug precursors which following administration, release the drug in vivo via some chemical or physiological process (e.g., a prodrug on being brought to the physiological pH or through enzyme action is converted to the desired drug form). Exemplary prodrugs upon cleavage release the corresponding free acid, and such hydrolyzable ester-forming residues of the compounds of the present invention include but are not limited to those having a carboxyl moiety wherein the free hydrogen is replaced by (C₁-C₄)alkyl, (C₂-C₇)alkanoyloxymethyl, 1-(alkanoyloxy)ethyl having from 4 to 9 carbon atoms, 1-methyl-1-alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms. N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N-(C₁-C₂)alkylamino(C₂-C₃)alkyl (such as β-dimethylaminoethyl), carbamoyl-C₁-C₂)alkyl, N,N-di(C₁-C₂)alkylcarbamoyl-(C₁-C₂)alkyl and piperidino-, pyrrolidino- or morpholino(C₂-C₃)alkyl.

The following paragraphs describe exemplary ring(s) for the generic ring descriptions contained herein.

Exemplary partially saturated, fully saturated or fully unsaturated three to eight membered rings optionally having one to four heteroatoms selected independently from oxygen, sulfur and nitrogen include cyclopropyl, cyclobutyl, cyclopentyl, cyclohexyl, cycloheptyl, cyclooctyl and phenyl. Further exemplary five membered rings include tetrazolyl, triazolyl, 2H-pyrrolyl, 3H-pyrrolyl, 2-pyrrolinyl, 3-pyrrolinyl, pyrrolidinyl, 1,3-dioxolanyl, oxazolyl, thiazolyl, imidazolyl, 2H-imidazolyl, 2-imidazolinyl, imidazolidinyl, pyrazolyl, 2-pyrazolinyl, pyrazolidinyl, isoxazolyl, isothiazolyl, 1,2-dithiolyl, 1,3-dithiolyl, 3H-1,2-oxathiolyl, 1,2,3-oxadiazolyl, 1,2,4-oxadiazolyl, 1,2,5-oxadiazolyl, 1,3,4-oxadiazolyl, 1,2,3-triazolyl, 1,2,4-triazolyl, 1,3,4-thiadiazolyl, 1,2,3,4-oxatriazolyl, 1,2,3,5-oxatriazolyl, 3H-1,2,3-dioxazolyl, 1,2,4-dioxazolyl, 1,3,2-dioxazotyl, 1,3,4-dioxazolyl, 5H-1,2,5-oxathiazolyl and 1,3-oxathiolyl.

Further exemplary six membered rings include 2H-pyranyl, 4H-pyranyl, pyridinyl, piperidinyl, 1,2-dioxinyl, 1,3-dioxinyl, 1,4-dioxanyl, morpholinyl, 1,4-dithianyl, thiomorpholinyl, pyridazinyl, pyrimidinyl, pyrazinyl, piperazinyl, 1,3,5-triazinyl, 1,2,4-triazinyl, 1,2,3-triazinyl, 1,3,5-trithianyl, 4H-1,2-oxazinyl, 2H-1,3-oxazinyl, 6H-1,3-oxazinyl, 6H-1,2-oxazinyl, 1,4-oxazinyl, 2H-1,2-oxazinyl, 4H-1,4-oxazinyl, 1,2,5-oxathiazinyl, 1,4-oxazinyl, o-isoxazinyl, p-isoxazinyl, 1,2,5-oxathiazinyl, 1,2,6-oxathiazinyl, 1,4,2-oxadiazinyl and 1,3,5,2-oxadiazinyl.

Further exemplary seven membered rings include azepinyl, oxepinyl, and thiepinyl.

Further exemplary eight membered rings include cyclooctyl, cyclooctenyl and cyclooctadienyl.

Exemplary partially saturated, fully saturated or fully unsaturated three to eight membered bicyclic ring systems optionally having one to four heteroatoms selected independently from oxygen, sulfur and nitrogen include naphthyl, tetrahydronaphthyl, indane, biphenyl ndolizinyl, indolyl, isoindolyl, 3H-indolyl, 1H-isoindolyl, indolinyl, cyclopenta(b)pyridinyl, pyrano(3,4-b)pyrrolyl, benzofuryl, isobenzofuryl, benzo(b)thienyl, benzo(c)thienyl, 1H-indazolyl, indoxazinyl, benzoxazolyl, benzimidazolyl, benzthiazolyl, purinyl, 4H-quinolizinyl, quinolinyl, isoquinolinyl, cinnolinyl, phthalazinyl, quinazolinyl, quinoxalinyl, 1,8-naphthyridinyl, pteridinyl, indenyl, isoindenyl, naphthyl, tetralinyl, decalinyl, 2H-1-benzopyranyl, pyrido(3,4-b)-pyridinyl, pyrido(3,2-b)-pyridinyl, pyrido(4,3-b)-pyridinyl, 2H-1,3-benzoxazinyl, 2H-1,4-benzoxazinyl, 1H-2,3-benzoxazinyl, 4H-3,1-benzoxazinyl, 2H-1,2-benzoxazinyl and 4H-1,4-benzoxazinyl.

By “halo” or “halogen” is meant chloro, bromo, iodo, or fluoro.

By “alkyl” is meant straight chain saturated hydrocarbon or branched chain saturated hydrocarbon. Exemplary of such alkyl groups (assuming the designated length encompasses the particular example) are methyl, ethyl, propyl, isopropyl, butyl, sec-butyl, tertiary butyl, isobutyl, pentyl, isopentyl, neopentyl, tertiary pentyl, 1-methylbutyl, 2-methylbutyl, 3-methylbutyl, hexyl, isohexyl, heptyl and octyl.

“Alkenyl” referred to herein may be linear or branched, and they may also be cyclic (e.g., cyclobutenyl, cyclopentenyl, cyclohexenyl) or bicyclic or contain cyclic groups. They contain 1-3 carbon-carbon double bonds, which may be cis or trans.

By “alkoxy” is meant straight chain saturated alkyl or branched chain saturated alkyl bonded through an oxy. Exemplary of such alkoxy groups (assuming the designated length encompasses the particular example) are methoxy, ethoxy, propoxy, isopropoxy, butoxy, isobutoxy, tertiary butoxy, pentoxy, isopentoxy, neopentoxy, tertiary pentoxy, hexoxy, isohexoxy, heptoxy and octoxy.

As used herein the term “mono-N-” or “di-N,N-C₁-Cₓ)alkyaminol” refers to the (C₁-Cₓ)alkyl moiety taken independently when it is di-N,N-C₁-Cₓ)alkyl (x refers to integers).

References (e.g. claim 1) to “said carbon” in the phrase “said carbon is optionally mono-, di- or tri-substituted independently with halo, said carbon is optionally mono-substituted with hydroxy, said carbon is optionally mono-substituted with oxo” refers to each of the carbons in the carbon chain including the connecting carbon.

References to a “nitrogen is optionally mono-, or disubstituted with oxo” herein (e.g. claim 1) refer to a terminal nitrogen which constitutes a nitro functionality.

It is to be understood that if a carbocyclic or heterocyclic moiety may be bonded or otherwise attached to a designated substrate through differing ring atoms without denoting a specific point of attachment, then all possible points are intended, whether through a carbon atom or, for example a trivalent nitrogen atom. For example, the term “pyridyl” means 2-, 3 or 4-pyridyl, the term “thienyl” means 2 or 3-thienyl, and so forth.

As used herein, the expressions “reaction-inert solvent” and “inert solvent” refer to a solvent or a mixture thereof which does not interact with starting materials, reagents, intermediates or products in a manner which adversely affects the yield of the desired product.

In one embodiment of the compounds of the present invention, R² is methyl, ethyl 2-propyl cyclopropyl, tert-butyl, or cyclobutyl, R⁴ is V⁰ optionally substituted with (R⁰)ₙ; and R⁵, R⁶R⁷, and R⁸ are each independently hydrogen, halogen, methyl, cyano, OCF₃, or CF₃.

In another embodiment, R⁴ is tetrazole or oxadiazole each optionally substituted with (C₁-C₄)alkyl wherein the (C₁-C₄)alkyl is optionally substituted with one to six fluorines.

In another embodiment, R² is ethyl or methyl; and R⁴ is 2-methyl-tetrazol-5-yl.

In another embodiment, R¹ is W—O—Y; and Y is methyl, ethyl, 1-propyl, 2-propyl or tert-butyl.

In another embodiment, R¹ is W—Y; Y is Z or (C₁-C₁₀)alkyl wherein said (C₁-C₁₀)alkyl substituent is optionally substituted with one, two or three substituents independently selected from halo, oxo, amino, amido, (C₁-C₆)alkoxy, carboxy, hydroxy and (C₁-C₆)alkyloxycarbonyl; and Z is (C₃-C₆)cycloalkyl optionally substituted independently with one or two oxo, amino, amido, carboxy, (C₁-C₆)alkoxy, or (C₁-C₆)alkyl, wherein said (C₁-C₆)alkyl substituent is optionally substituted with one, two or three substituents independently selected from halo, oxo, amino, amido, (C₁-C₆)alkoxy, carboxy, hydroxy and (C₁-C₆)alkyloxycarbonyl.

In another embodiment, R⁴ is Y; Y is (C₁-C₆)alkyl substituted with Z; and Z is (C₃-C₆)cycloalkyl optionally substituted independently with one or two oxo, amino, amido, carboxy, (C₁-C₆)alkoxy, or (C₃-C₆alkyl, wherein said (C₁-C₆)alkyl substituent is optionally substituted with one, two or three substituents independently selected from halo, oxo, amino, amido, (C₁-C₆)alkoxy, carboxy, hydroxy and (C₁-C₆)alkyloxycarbonyl.

In another embodiment, R² is ethyl or methyl; and Z is cyclohexyl optionally substituted with one or two amido, carboxy, (C₁-C₆)alkoxy, or (C₁-C₆)alkyl, wherein said (C₁-C₆)alkyl substituent is optionally substituted with one, two or three substituents selected from halo, oxo, amino, amido, (C₁-C₆)alkoxy, carboxy, hydroxy and (C₁-C₆)alkyloxycarbonyl.

In second embodiment of the compounds of the present invention, R² is methyl, ethyl, 2-propyl, cyclopropyl, tert-butyl, or cyclobutyl; R⁴ is —COO(C₁-C₄)alkyl, cyano, —CHO, —CONH₂, or —CO(C₁-C₄)alkyl; and R⁶, R⁶R⁷, and R⁸ are each independently hydrogen, halogen, methyl, cyano, OCF₃ or CF₃.

In another embodiment, R¹ is Y; and Z is present and Z is (C₃-C₆)cycloalkyl optionally substituted independently with one, two or three halo, hydroxy, amido, carboxy, (C₁-C₆)alkoxy, (C₁-C₆)alkyl, or (C₁-C₆)alkyloxycarbonyl, wherein said (C₁-C₆)alkyl substituent is optionally substituted with one, two or three substituents independently selected from halo, oxo, hydroxyl, amino, amido, (C₁-C₈)alkoxy, carboxy, and (C₁-C₆)alkyloxycarbonyl.

In another embodiment, Y is methyl, ethyl, 1-propyl, 2-propyl or tert-butyl, and Y is substituted with Z, and Z is cyclobutyl, cyclopentyl, or cyclohexyl, and Z is optionally substituted independently with one or two oxo, amino, amido, carboxy, (C₁-C₆)alkoxy, or (C₁-C₆)alkyl, wherein said (C₁-C₆)alkyl substituent is optionally substituted with one, two or three substituents independently selected from halo, oxo, amino, amido, (C₁-C₆)alkoxy, carboxy, hydroxy and (C₁-C₆)alkyloxycarbonyl.

In another embodiment R² is ethyl or methyl; and 4 is —COOCH₃, cyano, —CHO, —CONH₂, or —COCH₃.

In another embodiment, R¹ is W—Y; and Z is present and Z is (C₃-C₆)cycloalkyl optionally substituted independently with one, two or three halo, hydroxy, amido, carboxy, (C₁-C₆)alkoxy, (C₁-C₆)alkyl, or (C₁-C₆)alkyloxycarbonyl, wherein said (C₁-C₆)alkyl substituent is optionally substituted with one, two or three substituents independently selected from halo, oxo, amino, amido, (C₁-C₆)alkoxy, carboxy, hydroxy and (C₁-C₆)alkyloxycarbonyl.

In another embodiment, R² is ethyl or methyl; and Z is cyclohexyl optionally substituted with one or two amido, carboxy, (C₁-C₆)alkoxy, or (C₁-C₆)alkyl, wherein said (C₁-C₆)alkyl substituent is optionally substituted with one, two or three substituents selected from halo, oxo, amino, amido, (C₁-C₆)alkoxy, carboxy, hydroxy and (C₃-C₆)alkyloxycarbonyl.

In another embodiment, Z is cyclohexyl substituted with amido, carboxy or (C₁-C₆)alkyl, wherein said (C₁-C₆)alkyl substituent is optionally substituted with halo, oxo, amino, amido, carboxy, hydroxy, or (C₁-C₆)alkyloxycarbonyl.

In yet another embodiment, V⁰ is

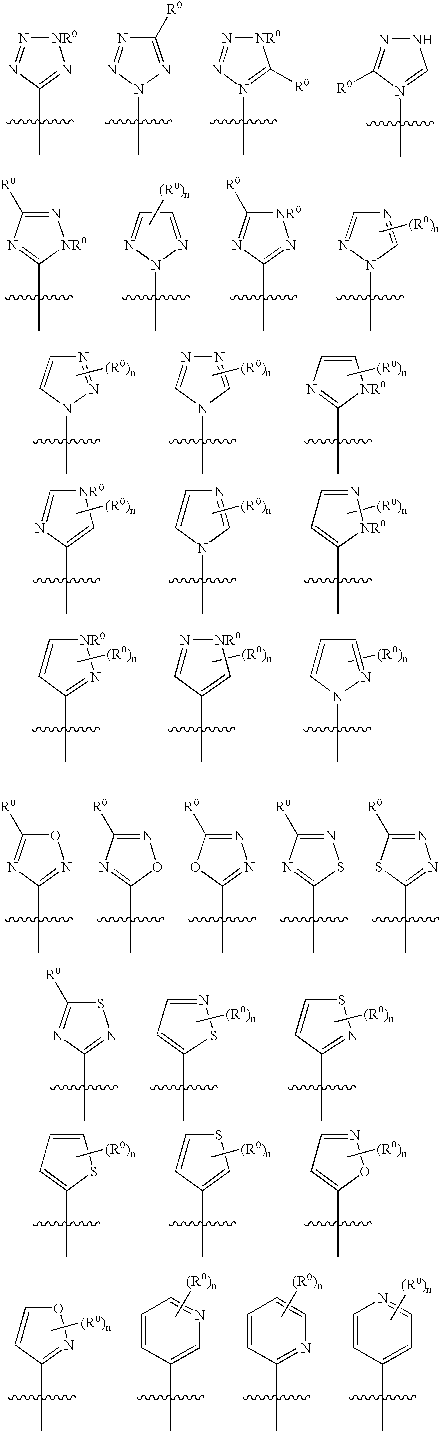

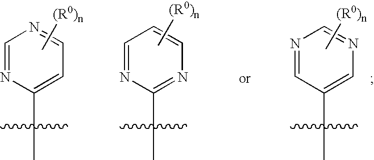

wherein each R⁰ is independently hydrogen, (C₁-C₃)alkyl, (C₁-C₃)alkoxy, hydroxy, or halo, wherein said (C₁-C₃)alkyl or (C₁-C₃)alkoxy is optionally independently substituted with one to nine halo or one hydroxy.

In another embodiment, V⁰ is

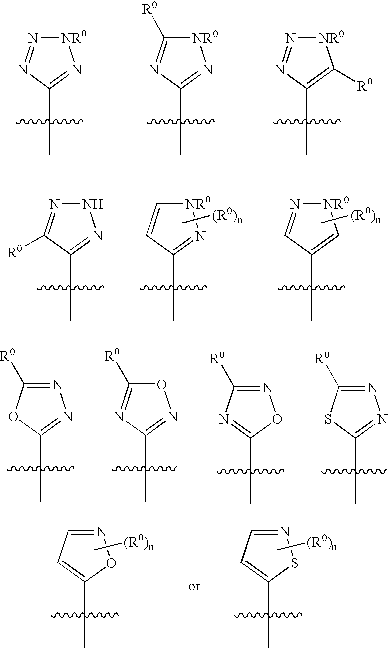

In another embodiment V⁰ is

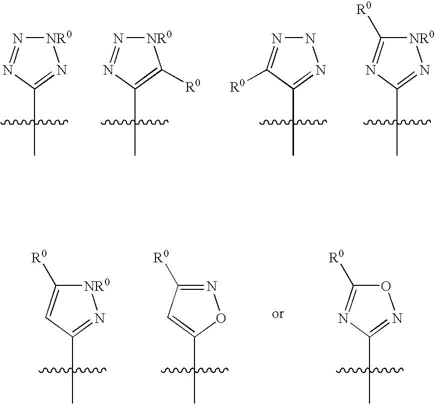

In another embodiment, V⁰ is 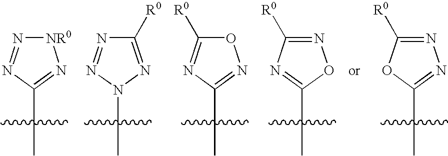
wherein each R⁰ independently hydrogen, (C₁-C₃)alkyl, (C₁-C₃)alkoxy, hydroxy, or halo, wherein said (C₁-C₃)alkyl or (C₁-C₃)alkoxy is optionally independently substituted with 1 to nine halo or one hydroxy.

In one embodiment of the method of the present invention, atherosclerosis is treated.

In another embodiment of the method of the present invention, peripheral vascular disease is treated.

In another embodiment of the method of the present invention, dyslipidemia is treated.

In another embodiment of the method of the present invention, hyperbetalipoproteinemia is treated.

In another embodiment of the method of the present invention, hypoalphalipoproteinemia is treated.

In another embodiment of the method of the present invention, familial-hypercholesterolemia is treated.

In another embodiment of the method of the present invention, coronary artery disease is treated.

In another embodiment of the method of the present invention, myocardial infarction is treated.

In one embodiment of the combination or kit of the present invention, the second compound is an HMG-CoA reductase inhibitor or a PPAR modulator.

In another embodiment of the combination or kit of the present invention, the second compound is fenofibrate, lovastatin, simvastatin, pravastatin, fluvastatin, atorvastatin, rivastatin, rosuvastatin or pitavastatin.

In another embodiment of the combination or kit of the present invention, the combination may comprise a cholesterol absorption inhibitor, wherein the cholesterol absorption inhibitor may be ezetimibe.

In another embodiment of the combination or kit of the present invention, said first compound is a compound of Formula III and said second compound is atorvastatin, or pharmaceutically acceptable salts thereof.

In one embodiment of the pharmaceutical composition, at least a major portion of the compound of claim 1 or 10 is amorphous, and the pharmaceutically acceptable vehicles diluent or carrier comprises at least one of a polymer and a substrate having a surface area of at least 20 m²/g. Moreover, the compound and the polymer may be in the form of a solid amorphous dispersion, or the compound is adsorbed onto said substrate. Furthermore, the polymer may comprise hydroxypropyl methylcellulose acetate succinate, hydroxypropyl methylcellulose, or polyvinylpyrrolidone.

The compounds of the present invention may have the advantage of having a pharmaceutically acceptable crystalline form. Furthermore, the compounds of the present invention may have the advantage of reduced hypertensive activity.

In general, the compounds of this invention may be made by processes which include processes analogous to those known in the chemical arts, particularly in light of the description contained herein. Certain processes for the manufacture of the compounds of this invention are provided as further features of the invention and are illustrated by the following reaction schemes. Other processes may be described in the experimental section.

Analogous processes are disclosed in the following U.S. patents, which are hereby incorporated by reference herein in their entirety; U.S. Pat. No. 6,140,342; U.S. Pat. No. 6,362,198; U.S. Pat. No. 6,147,090; U.S. Pat. No. 6,395,751; U.S. Pat. No. 6,147,089; U.S. Pat. No. 6,310,075. U.S. Pat. No. 6,197,786; U.S. Pat. No. 6,140,343. U.S. Pat. No. 6,489,478; and International Publication No. WO 00/17164.

The Reaction Schemes herein described are intended to provide a general description of the methodology employed in the preparation of many of the Examples given. However, it will be evident from the detailed descriptions given in the Experimental section that the modes of preparation employed extend further than the general procedures described herein. In particular, it is noted that the compounds prepared according to these Schemes may be modified further to provide new Examples within the scope of this invention. For example, an ester functionality may be reacted further using procedures well known to those skilled in the art to give another ester, an amide, a carbinol or a ketone.

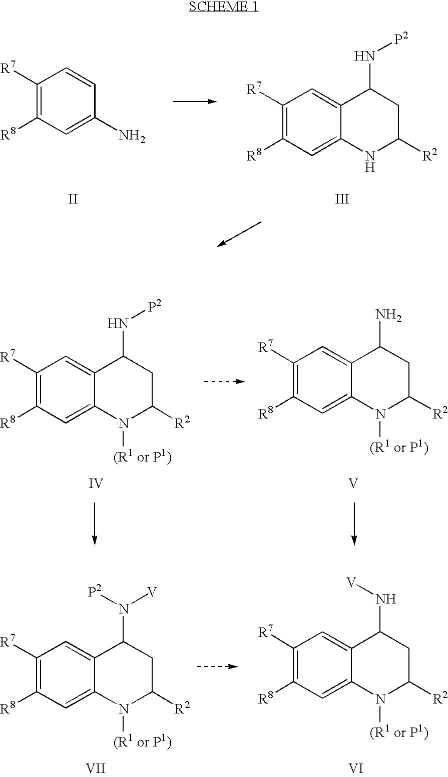

According to reaction Scheme 1, the desired compounds of Formula III wherein R², R⁷ and R⁸ are as described and P² is an appropriate protecting group may be prepared from the appropriate Formula II aromatic amine. The Formula III tetrahydroquinoline is prepared by treating the appropriate Formula II aromatic amine with the requisite carboxaldehyde in an inert solvent such as a hydrocarbon (e.g., hexanes, pentanes or cyclohexane), an aromatic hydrocarbon (e.g., benzene, toluene or xylene), a halocarbon (e.g., dichloromethane, chloroform, carbon tetrachloride or dichloroethane), an ether (e.g., diethyl ether, diisopropyl ether, tetrahydrofuran, tetrahydropyran, dioxane, dimethoxyethane, methyl tert-butyl ether, etc.), a nitrile (e.g., acetonitrile or propionitrile), a nitroalkane (e.g., nitromethane or nitrobenzene), preferably dichloromethane with a dehydrating agent (e.g., sodium sulfate or magnesium sulfate) at a temperature of about 0° C. to about 100° C. (preferably ambient temperature) for 1-24 hours (preferably 1 hour). The resulting solution is treated with a suitably substituted (e.g., benzyloxycarbonyl, t-butoxycarbonyl, methoxycarbonyl, formyl-, acetyl-, diallyl- or dibenzyl-), preferably carboxybenzyloxy-, N-vinyl species and with a Lewis acid (e.g., boron trifluoride, boron trifluoride etherate, zinc chloride, titanium tetrachloride, iron trichloride, aluminum trichloride, alkyl aluminum dichloride, dialkyl aluminum chloride or ytterbium (III) triflate; preferably boron trifluoride etherate) or a protic acid such as a hydrohalogenic acid (e.g., fluoro, chloro, bromo or iodo), an alkyl sulfonic acid (e.g.: p-toluene, methane or trifloromethane) or carboxylic acid (e.g., formic, acetic, trifluoroacetic or benzoic) at a temperature of from about −78° C. to about 50° C. (preferably ambient temperature) for 0.1 to 24 hours (preferably 1 hour).

Alternatively, the Formula II amine and appropriate carboxaldehyde may be condensed by treating a solution of the amine and an alkyl amine base (preferably triethylamine) in a polar aprotic solvent (preferably dichloromethane) with titanium tetrachloride in a polar aprotic solvent (preferably in dichloromethane) at a temperature between about −78° C. to about 40° C. (preferably 0° C.) followed by treatment with the carboxaldehyde at a temperature between about −78° C. to about 40° C. (preferably 0° C.). The reaction is allowed to proceed for about 0.1 to about 10 hours (preferably 1 hour) at a temperature between about 0° C. to about 40° C. (preferably room temperature) yielding the imine which is reacted with the N-vinyl species as above.

The compounds of Formula IV wherein R¹, R², R⁷, and R⁸ are as described above and P¹ and P² are protecting groups may be prepared from the corresponding Formula III amine by various amine reaction routes known to those skilled in the art. Thus, the Formula IV may be prepared from the corresponding Formula III tetrahydroquinoline employing standard methods for derivatizing amines into the functional groups described for R¹ above, see Richard Larock, Comprehensive Organic Transformations, VCH Publishers Inc., New York, 1989 and Jerry March, Advanced Organic Chemistry, John Wiley & Sons, New York, 1985. For example, a Formula III compound is treated with the appropriate carbonyl chloride, sulfonyl chloride, or sulfinyl chloride, isocyanate or thioisocyanate in a polar aprotic solvent (preferably dichloromethane) in the presence of a base (preferably pyridine) at a temperature of from about 78° C. to about 100° C. (preferably starting at 0° C. and letting warm to room temperature) for a period of 1 to 24 hours (preferably 12 hours).

Formula IV carbamate compounds (wherein R¹ is W—O—Y and W═C(O)) may be prepared from the Formula III amines via the corresponding carbamoyl chlorides by treating the Formula III amine with a phosgene solution in a hydrocarbon solvent (preferably toluene) at a temperature between about 0° C. and about 200° C. (preferably at reflux) for between 0.1 and 24 hours (preferably 2 hours). The corresponding carbamate may be prepared by treating a solution of the carbamoyl chlorides (prepared as described above) with the appropriate alcohol and a suitable base (preferably sodium hydride) in a polar solvent (preferably dioxane) at a temperature between about −78° C. and about 100° C. (preferably ambient temperature) for between 1 and 24 hours (preferably 12 hours.

Alternatively, the corresponding carbamate may be prepared by treating a solution of the carbamoyl chlorides at a temperature between about 0° C. and about 200° C. in the appropriate alcohol for between 1 and 240 hours (preferably 24 hours).

The Formula IV compound wherein R¹ is Y may be prepared using methods known to those skilled in the art to introduce Y substituents such as an alkyl or alkyl linked substituent. Methods include, for example, formation of the amide from the amine of Formula III and an activated carboxylic acid followed by reduction of the amide with borane in an etheral solvent such as tetrahydrofuran. Alternatively, the alkyl or alkyl inked substituent may be appended by reduction after condensing the amine of Formula III with the required carbonyl containing reactant. Also, the amine of Formula III may be reacted with the appropriate alkyl or aryl halide according to methods known to those skilled in the art.

Thus, the Formula III amine and an acid (e.g., halogenic, sulfuric, sulfonic or carboxylic, preferably acetic) are treated with the appropriate carbonyl containing reactant in a polar solvent (preferably ethanol) at a temperature of about 0° C. to about 100° C. (preferably room temperature) for about 0.1 to 24 hours (preferably 1 hour) followed by treatment with a hydride source (e.g., sodium borohydride, sodium cyanoborohydride, preferably sodium triacetoxyborohydride) at a temperature of about 0° C. to about 100° C. (preferably ambient temperature) for 0.1 to 100 hours (preferably 5 hours).

The Formula V amine wherein R¹, R², R⁷ and R⁸ are as described above and P¹ is a protecting group may be prepared from the corresponding Formula IV compound by deprotection (P²) using methods known to those skilled in the art, including hydrogenolysis, treatment with an acid (e.g., trifluoroacetic acid, hydrobromic), a base (sodium hydroxide), or reaction with a nucleophile (e.g. sodium methylthiolate, sodium cyanide, etc.) and for the trialkylsilyethoxy carbonyl group a fluoride is used (e.g. tetrabutyl ammonium fluoride). For removal of a benzyloxycarbonyl group, hydrogenolysis is performed by treating the Formula IV compound with a hydride source (e.g. 1 to 10 atmospheres of hydrogen gas, cyclohexene or ammonium formate) in the presence of a suitable catalyst (e.g., 5-20% palladium on carbon, palladium hydroxide: preferably 10% palladium on carbon) in a polar solvent (e.g., methanol, ethanol or ethyl acetate, preferably ethanol) at a temperature between about −78° C. and about 100° C., preferably ambient temperature, for 0.1 to 24 hours, preferably 1 hour.

The compounds of Formula VI of Scheme 1 wherein V is benzyl substituted with R⁵ and R⁶ as described above may be prepared from the corresponding Formula V amine by various amine reaction routes known to those skilled in the art including, for example, the methods described for the introduction of the R¹ substituent in the transformation of the compounds of Formula III to the compounds of Formula IV. Methods include, for example, formation of an amide from the amine of Formula V and an activated carboxylic acid followed by reduction of the amide with borane in an etheral solvent such as tetrahydrofuran. Alternatively, an alkyl or alkyl linked substituent may be appended by reduction of the appropriate imine, the imine being formed by condensing the amine of Formula V with the required carbonyl containing reactant. Also, the amine of Formula V may be reacted with the appropriate alkyl halide according to methods known to those skilled in the art.

Thus, the Formula V amine and an acid (e.g., halogenic, sulfuric, sulfonic or carboxylic, preferably hydrochloric) are treated with the appropriate carbonyl containing reagent in a polar solvent (preferably dichloromethane) at a temperature of about 0° C. to about 100° C. (preferably room temperature) for about 0.1 to 24 hours (preferably 1 hour) followed by treatment with a hydride source (e.g., sodium borohydride or sodium cyanoborohydride; preferably sodium triacetoxyborohydride) at a temperature of about 0° C. to about 100° C. (preferably ambient temperature) for 0.1 to 100 hours (preferably 5 hours)

The Formula VII compounds of Scheme 1 may be prepared from the corresponding Formula IV compound by methods known to those skilled in the art; for example, the methods described for the introduction of the V substituent above in the transformation of the Formula V compound to the Formula VI compound. Following this, the corresponding Formula VI compound may be prepared from the Formula VII compound by appropriate deprotection such as the methods described above for the transformation of the Formula IV compound to the Formula V compound.

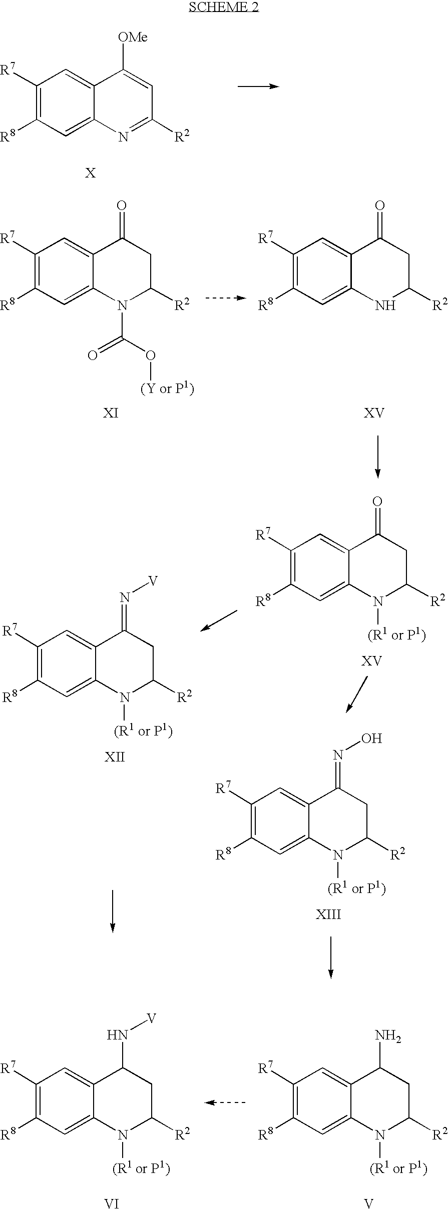

According to Scheme 2, the Formula XI dihydroquinolone compounds wherein R², R⁷, R⁸ and Y are as describe above, and P¹ is a protecting group, may be prepared from the corresponding Formula X quinolines by treatment with an organometallic species and a chloroformate followed by hydrolysis. Thus, a mixture of the Formula X quinoline and an excess (preferably 1.5 equivalents) of a organomagnesium species (Grignard reagent) in a polar aprotic solvent (e.g., diethyl ether or dichloromethane; preferably tetrahydrofuran) is treated with an excess (preferably 1.5 equivalents) of a Y or P¹-chloroformate at a temperature between about −100° C. and about 70° C. (preferably −78° C.) followed by warming to a temperature between about −0° C. and about 70° C. (preferably ambient temperature) for between 0.1 and 24 hours (preferably 1 hour). The resulting mixture is combined with an excess (preferably 2 equivalents) of an aqueous acid (preferably 1 molar hydrochloric acid) and mixed vigorously for between 0.1 and 24 hours (preferably 1 hour, or until hydrolysis of the intermediate enol ether is determined to be complete).

Of course, the Formula XI compounds are the Formula XVI compounds wherein R¹ is —C(O)OY or P¹ is —C(O)OP¹ without further transformation.

The Formula XV compounds may be prepared from the corresponding Formula XI dihydroquinolone (wherein the compound of Formula XI contains P¹) by appropriate deprotection (including spontaneous decarboxylation) as described for the transformation of the Formula IV compound to the Formula V compound.

The Formula XVI compounds wherein P¹ is a protecting group may be prepared from the corresponding Formula XV dihydroquinolone as described for the transformation of the Formula III compound to the Formula IV compound. In certain cases where the reagent has also reacted on the 4-position carbonyl oxygen, the substituent may be conveniently removed by treatment with acid (e.g., aqueous HCl) or base (e.g., aqueous sodium hydroxide).

The Formula VI amine compounds wherein V is benzyl substituted with R⁵ and R⁶ as described above may be prepared from the corresponding Formula XVI dihydroquinolone by a reductive amination sequence. The Formula XVI dihydroquinolone, an excess (preferably 1.1 equivalents) of an V-amine and an excess (preferably 7 equivalents) of an amine base (preferably triethylamine) in a polar solvent (preferably dichloromethane) are treated with 0.5 to 1.0 equivalents (preferably 0.55 equivalents) of titanium tetrachloride as a solution in a suitable polar solvent (preferably dichloromethane) at a temperature between about 0° C. and about 40-C (preferably ambient temperature) for between 1 to 24 hours (preferably 12 hours). The resulting Formula XIII imine is reduced by treatment with a reducing agent (preferably sodium borohydride) in an appropriate polar solvent (preferably ethanol) at a temperature between about 0° C. and about 80° C. (preferably room temperature) for between 1 and 24 hours (preferably 12 hours) resulting in a mixture of diastereomeric Formula VI amines, generally favoring the trans isomer. Alternatively, the reduction may be performed by treating the Formula XII imine directly with an excess (preferably 5 equivalents) of zinc borohydride as a solution in ether (preferably 0.2 molar) at a temperature between about 0° C. and about 40° C. (preferably ambient temperature) for between 1 and 24 hours (preferably 12 hours) resulting in a mixture of diastereomeric Formula VI, amines, generally favoring the cis isomer.

Alternatively, the Formula VI amine may be prepared from the corresponding Formula XVI dihydroquinolones by formation of an oxime, reduction and substitution of the amine. Thus, the Formula XVI dihydroquinolone, excess (preferably 3 equivalents) hydroxylamine hydrochloride and an excess (preferably 2.5 equivalents) of base (preferably sodium acetate) are reacted at a temperature between about 0° C. and about 100° C. (preferably at reflux) for between 1 and 24 hours (preferably 2 hours) in a polar solvent (preferably ethanol). The resulting Formula XIII oxime is treated with excess (preferably 6 equivalents) aqueous base (preferably 2N potassium hydroxide) in a polar solvent (preferably ethanol) and an excess (preferably 4 equivalents) of a nickel-aluminum alloy (preferably 1:1 by weight) at a temperature between about 0° C. and about 100° C. (preferably ambient temperature) for between 0.25 and 24 hours (preferably 1 hour). The resulting Formula V amine is obtained as a diastereomeric mixture (generally favoring the cis isomer). The Formula VI secondary amine may be prepared from the appropriate Formula V amine as described in Scheme 1 for the transformation of the Formula V compound to the Formula VI compound.

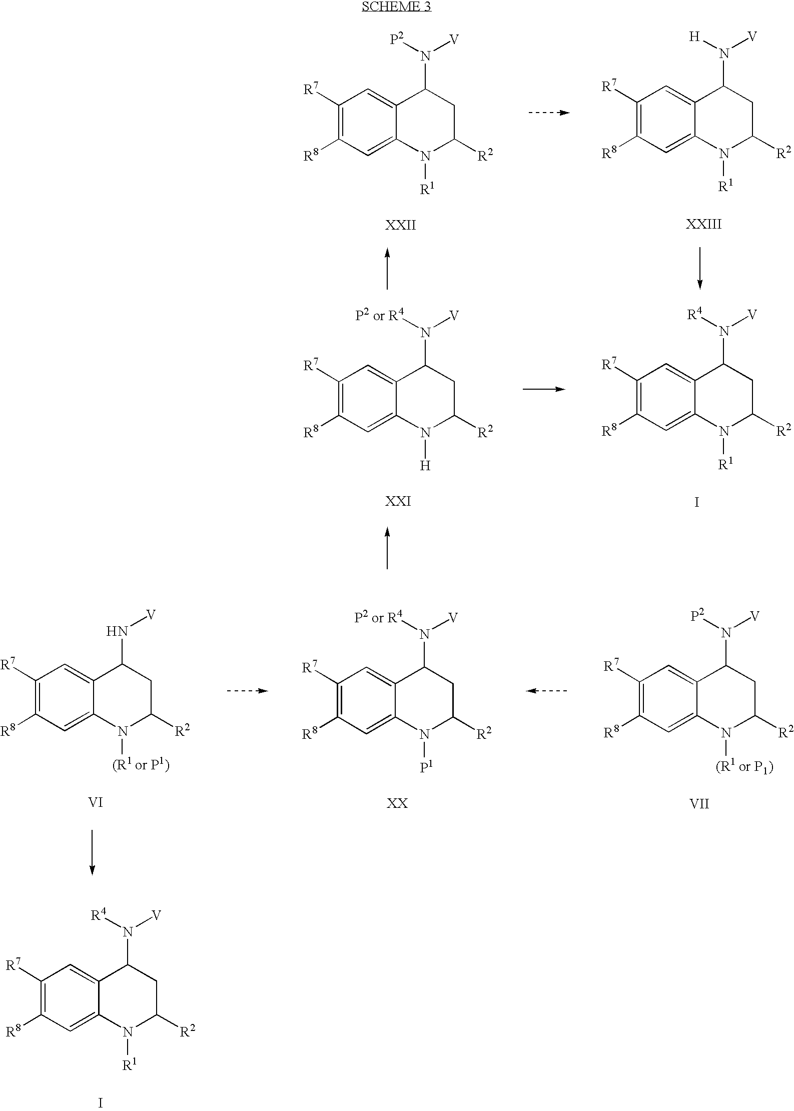

According to Scheme 3, the Formula I compounds wherein V is benzyl substituted with R⁵ and R⁶, and R¹, R², R⁴, P⁷, and R⁸ are as described above may be prepared from the appropriate Formula VI compounds using methods known to those skilled in the art, including, for example, the methods described for the introduction of the R¹ substituent in the transformation of the compounds of Formula III to the compounds of Formula IV.

Alternatively, according to Scheme 3, where appropriate, if the functionality at R¹ is incompatible with the reaction to form the Formula I compound, then the P¹ protected Formula VI compound may be transformed to the Formula I compound through protection/deprotection sequences and introduction of the desired substituents. Thus, the Formula VI is treated with the appropriate reagent protecting group precursor, activated carbonate (e.g., chloroformate, dicarbonate or carbonyl imidazole)) in a polar solvent (preferably dichloromethane) in the presence of an excess of amine base (preferably pyridine) at a temperature between about −20° C. and about 40° C. (preferably ambient temperature) for between 1 and 24 hours (preferably 12 hours) to yield the Formula XX compound.

Also, the Formula XX compounds, herein P² is a protecting group may be obtained as shown in Scheme I for the Formula VII compounds (having P¹).

The Formula XXI amines may be prepared from the Formula XX compound by selective deprotection of P¹. When P¹ is, for example, t-butoxycarbonyl, the Formula XXI compound is conveniently prepared by treatment with an acid (preferably trifluoroacetic acid) at a temperature between about 0° C. and 100° C. (preferably room temperature) for 0.1 to 24 hours (preferably 1 hour).

The compounds of Formula I or compounds of Formula XXII may be prepared from the corresponding Formula XXI amine (wherein R⁴ or P² is present respectively) by various amine reaction routes known to those skilled in the art, for example, those described in Scheme I for the transformation of the Formula III compound to the Formula IV compound.

The Formula XXIII amines may be prepared from the Formula XXII compounds by suitable deprotection. When P² is, for example, benzyloxycarbonyl, the Formula XXIII compound is prepared by treatment with an excess of a hydride source (e.g., cyclohexene, hydrogen gas or preferably ammonium formate) in the presence of 0.01 to 2 equivalents (preferably 0.1 equivalent) of a suitable catalyst (preferably 10% palladium on carbon) in a polar solvent (preferably ethanol) at a temperature between about 0° C. and about 100° C. (preferably room temperature) for 0.1 to 24 hours (preferably 1 hour).

The Formula I compound wherein R⁴ is as described above may be prepared using the methods described for the conversion of the Formula VI compound to the Formula I compound in Scheme 3 above.

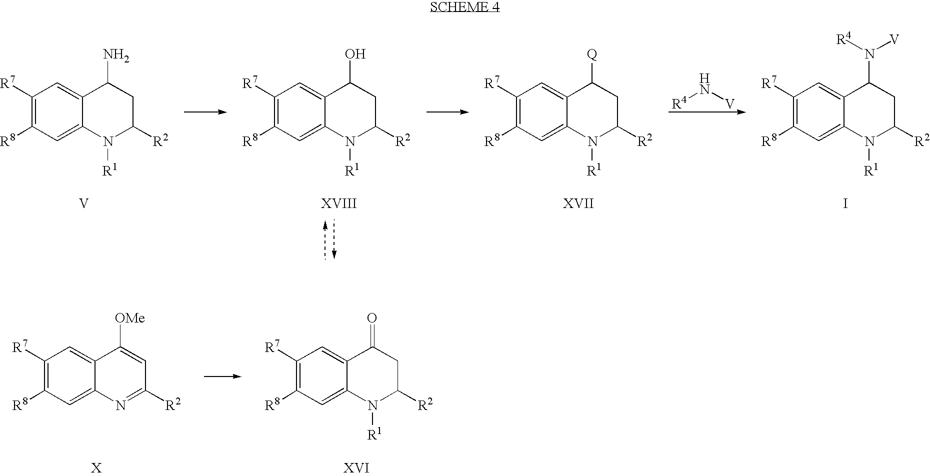

According to reaction Scheme 4, the desired compounds I wherein R¹, R², R⁴, R⁷, and R⁸, are as defined above, and V is benzyl substituted with R⁵ and R⁶ as defined above, may be prepared as a mixture of diastereoisomers from the corresponding Formula XVII compounds by reaction with a compound VNHR⁴ in the presence of a suitable base such as 1,8-diazabicyclo[5.4.0]undec-7-ene, diisopropylethylamine, triethylamine or sodium hydride in a reaction inert solvent each as N,N-dimethylformamide, dimethylsulfoxide, acetonitrile or toluene at a temperature between 0° C. to 60° C., typically ambient.

The desired Formula XVII compounds of Scheme 4 wherein Q is a leaving group such as chlorine, bromine, methanesulfonyloxy or p-toluenesulfonyloxy may be prepared as a mixture of diastsereoisomers from the corresponding Formula XVIII compounds by reaction with the appropriate reagent such as methanesulfonyl chloride or toluenesulfonyl chloride in the presence of a suitable base such as disopropylethylamine or triethylamine in a reaction inert solvent such as N,N-dimethylformamide, dimethylsulfoxide, chloroform, methylene chloride or toluene at a temperature between 0° C. to 60° C., typically ambient. Other suitable reagents for formation of the Formula XVII compounds include phosphorus (III) chloride, phosphorus (III) bromide and thionyl chloride optionally in a reaction inert solvent such as chloroform, methylene chloride, pyridine or toluene at a temperature between 0° C. to 60° C., typically ambient. The desired Formula XVIII compounds of Scheme 4 may be prepared as a mixture of diastereoisomers from the corresponding Formula XVI compounds by reduction of the carbonyl group using methods and reagents well known to those skilled in the arts, such as can be found in L. A. Paquette (Ed), Encyclopedia of Reagents for Organic Synthesis, John Wiley and Sons, Chichester, England. 1995, for example using sodium borohydride in an alcohol solvent such as methanol of ethanol at a temperature between 0° C. to 60° C., typically ambient or using potassium tri-sec-butylborohydride (K-Selectride®) in a reaction inert solvent such as tetrahydrofuran or diethyl ether at a temperature between −78° C. to 25° C., typically 0° C.

In an alternative procedure, the desired Formula XVIII compounds may be obtained by treatment of the corresponding Formula V compounds with sodium nitrite in the presence of an acid, preferably acetic acid followed by hydrolysis with a suitable base such as lithium, sodium, or potassium hydroxide, preferably sodium hydroxide in a suitable hydroxylic solvent such as ethanol to give the desired Formula XVIII compounds. Methods for the preparation of Formula V compounds are described in U.S. Pat. No. 6,197,786 and international Application WO 0140190.

The desired Formula XVI compounds of Scheme 4 wherein R¹ is an alkoxycarbonyl group may be prepared from the corresponding 4-methoxyquinoline compounds of Formula X by treatment with an organomagnesium derivative of the R² group together with an acylating agent such as ethyl chloroformate at a temperature between −100° C. to 70° C., typically −78° C. in a reaction inert solvent such as tetrahydrofuran followed by warming to a temperature between 0° C. and about 70° C. (preferably ambient) for between 0.1 and 24 hr, preferably 1 hr, followed by hydrolysis in aqueous acid, preferably 1N hydrochloric acid to give the desired Formula IX compounds, as described in U.S. Pat. No. 6,197,786.

In an alternative procedure, the desired Formula XVI compounds may be obtained by oxidation of the corresponding Formula XVIII compounds using a variety of methods and reagents well known to those skilled in the arts, such as can be found in L. A. Paquette (Ed), Encyclopedia of Reagents for Organic Synthesis, John Wiley and Sons, Chichester, England, 1995, for example pyridinium chlorochromate and aqueous sodium hypochlorite in the presence of a catalytic amount of 2,2,6,6-tetramethyl-1-piperidinyloxy (TEMPO) free radical and catalytic potassium bromide in a suitable reaction inert solvent such as methylene chloride, or alternatively with acetic anhydride and dimethylsulfoxide.

As an initial note, in the preparation of compounds, it is noted that some of the preparation methods useful for the preparation of the compounds described herein may require protection of remote functionality (e.g., primary amine, secondary amine, carboxyl in intermediates). The need for such protection will vary depending on the nature of the remote functionality and the conditions of the preparation methods. The need for such protection is readily determined by one skilled in the art. The use of such protection/deprotection methods is also within the skill in the art. For a general description of protecting groups and their use, see T. W. Greene, Protective Groups in Organic Synthesis, John Wiley & Sons, New York, 1991.

For example, in the reaction schemes, certain compounds contain primary amines or carboxylic acid functionalities which may interfere with reactions at other sites of the molecule if left unprotected. Accordingly, such functionalities may be protected by an appropriate protecting group which may be removed in a subsequent step. Suitable protecting groups for amine and carboxylic acid protection include those protecting groups commonly used in peptide synthesis (such as N-t-butoxycarbonyl, benzyloxycarbonyl, and 9-fluorenylmethylenoxycarbonyl for amines and lower alkyl or benzyl esters for carboxylic acids) which are generally not chemically reactive under the reaction conditions described and can typically be removed without chemically altering other functionality in the compound.

Prodrugs of the compounds of the present invention may be prepared according to methods known to those skilled in the art. Exemplary processes are described below.

Prodrugs of this invention where a carboxyl group in a carboxylic acid of the compounds is replaced by an ester may be prepared by combining the carboxylic acid with the appropriate alkyl halide in the presence of a base such as potassium carbonate in an inert solvent such as dimethylformamide at a temperature of about 0 to 100° C. for about 1 to about 24 hours. Alternatively the acid is combined with an appropriate alcohol as solvent in the presence of a catalytic amount of acid such as concentrated sulfuric acid at a temperature of about 20 to 100° C., preferably at a reflux, for about 1 hour to about 24 hours. Another method is the reaction of the acid with a stoichiometric amount of the alcohol in the presence of a catalytic amount of acid in an inert solvent such as toluene or tetrahydrofuran, with concomitant removal of the water being produced by physical (e.g. Dean-Stark trap) or chemical (e.g., molecular sieves) means.

Prodrugs of this invention where an alcohol function has been derivatized as an ether may be prepared by combining the alcohol with the appropriate alkyl bromide or iodide in the presence of a base such as potassium carbonate in an inert solvent such as dimethylformamide at a temperature of about 0 to 100° C. for about 1 to about 24 hours. Alkanoylaminomethyl ethers may be obtained by reaction of the alcohol with a bis-(alkanoylamino)methane in the presence of a catalytic amount of acid in an inert solvent such as tetrahydrofuran, according to a method described in U.S. Pat. No. 4,997,984. Alternatively, these compounds may be prepared by the methods described by Hoffman et al. in J. Org. Chem. 1994, 59, 3530.

Glycosides are prepared by reaction of the alcohol and a carbohydrate in an inert solvent such as toluene in the presence of acid. Typically the water formed in the reaction is removed as it is being formed as described above. An alternate procedure is the reaction of the alcohol with a suitably protected glycosyl halide in the presence of base followed by deprotection.

N-(1-hydroxyalkyl)amides, N-(1-hydroxy-1-(alkoxycarbonyl)methyl)amides may be prepared by the reaction of the parent amide with the appropriate aldehyde under neutral or basic conditions (e.g., sodium ethoxide in ethanol) at temperatures between 25 and 70° C. N-alkoxymethyl or N-1-(alkoxy)alkyl derivatives can be obtained by reaction of the N-unsubstituted compound with the necessary alkyl halide in the presence of a base in an inert solvent.

The compounds of this invention may also be used in conjunction with other pharmaceutical agents (e.g., LDL-cholesterol lowering agents, triglyceride lowering agents) for the treatment of the disease/conditions described herein. For example, they may be used in combination with a HMG-CoA reductase inhibitor, a cholesterol synthesis inhibitor, a cholesterol absorption inhibitor, another CETP inhibitor, a MTP/Apo B secretion inhibitor, a PPAR modulator and other cholesterol lowering agents such as a fibrate, niacin, an ion-exchange resin, an antioxidant, an ACAT inhibitor, and a bile acid sequestrant. Other pharmaceutical agents would also include the following: a bile acid reuptake inhibitor, an ileal bile acid transporter inhibitor, an ACC inhibitor, an antihypertensive (such as NORVASC®), a selective estrogen receptor modulator, a selective androgen receptor modulator, an antibiotic, an antidiabetic (such as metformin, a PPARγ activator, a sulfonylurea, insulin, an aldose reductase inhibitor (ARI) and a sorbitol dehydrogenase inhibitor (SDI)), and aspirin (acetylsalicylic acid or a nitric oxide releasing asprin). A slow-release form of niacin is available and is known as Niaspan. Niacin may also be combined with other therapeutic agents such as statins, i.e. lovastatin, which an HMG-CoA reductase inhibitor and described further below. This combination therapy is known as ADVICOR® (Kos Pharmaceuticals Inc.) In combination therapy treatment, both the compounds of this invention and the other drug therapies are administered to mammals (e.g., humans, male or female) by conventional methods.

Any HMG-CoA reductase inhibitor may be used in the combination aspect of this invention. The term HMG-CoA reductase inhibitor refers to compounds which inhibit the bioconversion of hydroxymethylglutaryl-coenzyme A to mevalonic acid catalyzed by the enzyme HMG-CoA reductase. Such inhibition is readily determined by those skilled in the art according to standard assays (e.g., Meth. Enzymol. 1981; 71:455-509 and references cited therein). A variety of these compounds are described and referenced below however other HMG-CoA reductase inhibitors will be known to those skilled in the art. U.S. Pat. No. 4,231,938 (the disclosure of which is hereby incorporated by reference) discloses certain compounds isolated after cultivation of a microorganism belonging to the genus Aspergillus, such as lovastatin. Also, U.S. Pat. No. 4,444,784 (the disclosure of which is hereby incorporated by reference) discloses synthetic derivatives of the aforementioned compounds, such as simvastatin. Also, U.S. Pat. No. 4,739,073 (the disclosure of which is incorporated by reference) discloses certain substituted indoles, such as fluvastatin. Also, U.S. Pat. No. 4,346,227 (the disclosure of which is incorporated by reference) discloses ML-236B derivatives, such as pravastatin. Also, EP-491226A (the disclosure of which is incorporated by reference) discloses certain pyridyldihydroxyheptenoic acids, such as cerivastatin. In addition, U.S. Pat. No. 5,273,995 (the disclosure of which is incorporated by reference) discloses certain 6-[2-(substituted-pyrrol-1-yl)alkyl]pyran-2-ones such as atorvastatin and any pharmaceutically acceptable form thereof (i.e. LIPITOR®). Additional HMG-CoA reductase inhibitors include rosuvastatin and pitavastatin. Statins also include such compounds as rosuvastatin disclosed in U.S. RE37,314 E, pitavastatin disclosed in EP 304063 B1 and U.S. Pat. No. 5,011,930; mevastatin, disclosed in U.S. Pat. No. 3,983,140, which is incorporated herein by reference; velostatin, disclosed in U.S. Pat. No. 4,448,784 and U.S. Pat. No. 4,450,171, both of which are incorporated herein by reference; compactin, disclosed in U.S. Pat. No. 4,804,770, which is incorporated herein by reference; dalvastatin, disclosed in European Patent Application Publication No. 738510 A2 fluindostatin, disclosed in European Patent Application Publication No. 363934 A1; and dihydrocompactin, disclosed in U.S. Pat. No. 4,450,171, which is incorporated herein by reference.

Any PPAR modulator may be used in the combination aspect of this invention. The term PPAR modulator refers to compounds which modulate peroxisome proliterator activator receptor (PPAR) activity in mammals, particularly humans. Such modulation is readily determined by those skilled in the art according to standard assays known in the literature. It is believed that such compounds, by modulating the PPAR receptor, regulate transcription of key genes involved in lipid and glucose metabolism such as those in fatty acid oxidation and also those involved in high density lipoprotein (HDL) assembly (for example, apolipoprotein Al gene transcription), accordingly reducing whole body fat and increasing HDL cholesterol. By virtue of their activity, these compounds also reduce plasma levels of triglycerides, VLDL cholesterol, LDL cholesterol and their associated components such as apolipoprotein B in mammals, particularly humans, as well as increasing HDL cholesterol and apolipoprotein Al. Hence, these compounds are useful for the treatment and correction of the various dyslipidemias observed to be associated with the development and incidence of atherosclerosis and cardiovascular disease, including hypoalphalipoproteinemia and hypertriglyceridemia. A variety of these compounds are described and referenced below, however, others will be known to those skilled in the art International Publication Nos. WO 02/064549 and 02/064130 and U.S. patent application Ser. No. 10/720,942, filed Nov. 24, 2003; U.S. patent application Ser. No. 11/012139 filed Dec. 16, 2004 and U.S. patent application Ser. No. 11/065774 filed Feb. 24, 2005 (the disclosures of which are hereby incorporated by reference) disclose certain compounds which are PPARα activators.

Any other PPAR modulator may be used in the combination aspect of this invention. In particular, modulators of PPARβ and/or PPARγ may be useful incombination with compounds of the present invention. An example PPAR inhibitor is described in US2003/0225158 as {5-Methoxy-2-methyl-4-[4-(4-trifluoromethyl-benzyloxy)-benzylsulfany]-phenoxy}-acetic acid.

Any MTP/Apo B (microsomal triglyceride transfer protein and or apolipoprotein B) secretion inhibitor may be used in the combination aspect of this invention. The term MTP/Apo B secretion inhibitor refers to compounds which inhibit the secretion of triglycerides, cholesteryl ester, and phospholipids. Such inhibition is readily determined by those skilled in the art according to standard assays (e.g., Wetterau, J. R. 1992; Science 258-999). A variety of these compounds are described and referenced below however other MTP/Apo B secretion inhibitors will be known to those skilled in the art, including imputapride (Bayer) and additional compounds such as those disclosed in WO 95/40640 and W/O 98/23593, (two exemplary publications).

For example, the following MTP/Apo B secretion inhibitors are particularly useful.
4′-trifluoromethyl-biphenyl-2-carboxylic acid[2-(1H-[1,2,4]triazol-3-ylmethyl)-1,2,3,4-tetrahydro-isoquinolin-6-yl]-amide;
4′-trifluoromethyl-biphenyl-2-carboxylic acid[2-(2-acetylamino-ethyl)-1,2,3,4-tetrahydro-isoquinolin-6-yl]-amide;
(2-{6-[(4′-trifluoromethyl-biphenyl-2-carbonyl)-amino]-3,4-dihydro-1H-isoquinolin-2-yl}-ethyl) -carbamic acid methyl ester;
4′-trifluoromethyl-biphenyl-2-carboxylic acid[2-(1H-imidazol-2-ylmethyl)-1,2,3,4-tetra hydro-isoquinolin-6-yl]-amide;
4′-trifluoromethyl-biphenyl-2-carboxylic acid[2-2,2-diphenyl-ethyl)-1,2,3,4-tetrahydro-isoquinolin-6-yl]-amide;
4′-trifluoromethyl-biphenyl-2-carboxylic acid[2-(2-ethoxy-ethyl)-1,2,3,4-tetrahydro-isoquinolin-6-yl]-amide;
(S)-N-{2-[benzyl(methyl)-amino]-2-oxo-1-phenylethyl)-1-met}yl-5-[4′-(trifluoromethyl)[1,1′-biphenyl]-2-carboxamido]-1H-indole-2-carboxamide;
(S)-2-[(4′-Trifluoromethyl-biphenyl-2-carbonyl)-amino]-quinoline-6-carboxylic acid (pentylcarbamoyl-phenyl-methyl)-amide;
1H-indole-2-carboxamide, 1-methyl-N-[(1S)-2-[methyl(phenylmethyl)amino]-2-oxo-1-phenylethyl]-5-[[[4′-(trifluoromethyl)[1,1-biphenyl]-2-yl]carbonyl]amino]; and
N-[(1S)-2-(benzylmethylamino)-2-oxo-1-phenylethyl]-1-methyl-5-[[[4′-(trifluoromethyl)biphenyl-2-yl]carbonyl]amino]-1H-indole-2-carboxamide.

Any HMG-CoA synthase inhibitor may be used in the combination aspect of this invention. The term HMG-CoA synthase inhibitor refers to compounds which inhibit the biosynthesis of hydroxymethylglutaryl-coenzyme A from acetyl-coenzyme A and acetoacetyl-coenzyme A, catalyzed by the enzyme HMG-CoA synthase. Such inhibition is readily determined by those skilled in the an according to standard assays (Meth Enzymol. 1975; 35:155-160: Meth. Enzymol. 1985; 110:19-26 and references cited therein). A variety of these compounds are described and referenced below, however other HMG-CoA synthase inhibitors will be known to those skilled in the art. U.S. Pat. No. 5,120,729 (the disclosure of which is hereby incorporated by reference) discloses certain beta-lactam derivatives. U.S. Pat. No. 5,064,856 (the disclosure of which is hereby incorporated by reference) discloses certain spiro-lactone derivatives prepared by culturing a microorganism (MF5253). U.S. Pat. No. 4,847,271 (the disclosure of which is hereby incorporated by reference) discloses certain oxetane compounds such as 11-(3-hydroxymethyl-4-oxo-2-oxetayl)-3,5,7-trimethyl-2,4-undeca-dienoic acid derivatives.

Any compound that decreases HMG-CoA reductase gene expression may be used in the combination aspect of this invention. These agents may be HMG-CoA reductase transcription inhibitors that block the transcription of DNA or translation inhibitors that prevent or decrease translation of mRNA coding for HMG-CoA reductase into protein. Such compounds may either affect transcription or translation directly, or may be biotransformed to compounds that have the aforementioned activities by one or more enzymes in the cholesterol biosynthetic cascade or may lead to the accumulation of an isoprene metabolite that has the aforementioned activities. Such compounds may cause this effect by decreasing levels of SREBP (sterol receptor binding protein) by inhibiting the activity of site-1 protease (S1P) or agonizing the oxzgenal receptor or SCAP. Such regulation is readily determined by those skilled in the art according to standard assays (Meth. Enzymol. 1985; 110:9-19). Several compounds are described and referenced below, however other inhibitors of HMG-CoA reductase gene expression will be known to those skilled in the art. U.S. Pat. No. 5,041,432 (the disclosure of which is incorporated by reference) discloses certain 15-substituted lanosterol derivatives. Other oxygenated sterols that suppress synthesis of HMG-CoA reductase are discussed by E.I. Mercer (Prog. Lip. Res. 1993;32:357-416).

Any compound having activity as a CETP inhibitor can serve as the second compound in the combination therapy aspect of the present invention. The term CETP inhibitor refers to compounds that inhibit the cholesteryl ester transfer protein (CETP) mediated transport of various cholesteryl esters and triglycerides from HDL to LDL and VLDL. Such CETP inhibition activity is readily determined by those skilled in the art according to standard assays (e.g., U.S. Pat. No. 6,140,343). A variety of CETP inhibitors will be known to those skilled in the art, for example, those disclosed in commonly assigned U.S. Pat. No. 6,140,343 and commonly assigned U.S. Pat. No. 6,197,786. CETP inhibitors disclosed in these patents include compounds, such as [2R,4S]4-[(3,5-bis-trifluoromethyl-benzyl)-methoxycarbonyl-amino]-2-ethyl-8-trifluoromethyl-3,4-dihydro-2H-quinoline-1-carboxylic acid ethyl ester, which is also known as torcetrapib. CETP inhibitors are also described in U.S. Pat. No. 6,723,752, which includes a number of CETP inhibitors including (2R)-3-{[3-(4-Chloro-ethyl-phenoxy)-phenyl]-[[3-(1,1,2,2-tetrafluoro-ethoxy)-phenyl]-methyl]-amino}-1,1,1-trifluoro-2-propanol. Moreover, CETP inhibitors included herein are also described in U.S. patent application Ser. No. 10/807,838 filed Mar. 23, 2004. U.S. Pat. No. 5,512,548 discloses certain polypeptide derivatives having activity as CETP inhibitors, while certain CETP-inhibitory rosenonolactone derivatives and phosphate-containing analogs of cholesteryl ester are disclosed in J. Antibiot, 49(8): 815-816 (1996), and Bioorg. Med. Chem. Lett.; 6:1951-1954 (1996), respectively.

Any squalene synthetase inhibitor may be used in the combination aspect of this invention. The term squalene synthetase inhibitor refers to compounds which inhibit the condensation of 2 molecules of farnesylpyrophosphate to form squalene, catalyzed by the enzyme squalene synthetase. Such inhibition is readily determined by those skilled in the art according to standard assays (Meth. Enzymol. 1969, 15: 393-454 and Meth. Enzymol. 1985; 110:359-373 and references contained therein). A variety of these compounds are described in and referenced below however other squalene synthetase inhibitors will be known to those skilled in the art. U.S. Pat. No. 5,026,554 (the disclosure of which is incorporated by reference) discloses fermentation products of the microorganism MF5465 (ATCC 74011) including zaragozic acid. A summary of other patented squalene synthetase inhibitors has been compiled (Curr. Op. Ther. Patents (1993) 861-4).

Any squalene epoxidase inhibitor may be used in the combination aspect of this invention. The term squalene epoxidase inhibitor refers to compounds which inhibit the bioconversion of squalene and molecular oxygen into squalene-2,3-epoxide, catalyzed by the enzyme squalene epoxidase. Such inhibition is readily determined by those skilled in the art according to standard assays (Biochim. Biophys. Acta 1984; 794:466-471). A variety of these compounds are described and referenced below, however other squalene epoxidase inhibitors will be known to those skilled in the art. U.S. Pat. Nos. 5,011,859 and 5,064,859 (the disclosures of which are incorporated by reference) disclose certain fluoro analogs of squalene. EP publication 395,768 A (the disclosure of which is incorporated by reference) discloses certain substituted allylamine derivatives. PCT publication WO 9312069 A (the disclosure of which is hereby incorporated by reference) discloses certain amino alcohol derivatives. U.S. Pat. No. 5,051,514 (the disclosure of which is hereby incorporated by reference) discloses certain cyclopropyloxy-squalene derivatives.

Any squalene cyclase inhibitor may be used as the second component in the combination aspect of this invention. The term squalene cyclase inhibitor refers to compounds which inhibit the bioconversion of squalene-2,3-epoxide to lanosterol, catalyzed by the enzyme squalene cyclase. Such inhibition is readily determined by those skilled in the art according to standard assays (FEBS Lett. 1989; 244:347-350). In addition, the compounds described and referenced below are squalene cyclase inhibitors, however other squalene cyclase inhibitors will also be known to those skilled in the art. PCT publication WO9410150 (the disclosure of which is hereby incorporated by reference) discloses certain 1,2,3,5,6,7,8,8a-octahydro-5,5,8(beta)-trimethyl-6-isoquinolineamine derivatives, such as N-trifluoroacetyl-1,2,3,5,6,7,8,8a-octahydro-2-allyl-5,5,8(beta)-trimethyl-6(beta)-isoquinolineamine. French patent publication 2697250 (the disclosure of which is hereby incorporated by reference) discloses certain beta, beta-dimethyl-4-piperidine ethanol derivatives such as 1-(1,5,9-trimethyldecyl)-beta, beta-dimethyl-4-piperidineethanol

Any combined squalene epoxidase/squalene cyclase inhibitor may be used as the second component in the combination aspect of this invention. The term combined squalene epoxidase/squalene cyclase inhibitor refers to compounds that inhibit the bioconversion of squalene to lanosterol via a squalene-2,3-epoxide intermediate. In some assays it is not possible to distinguish between squalene epoxidase inhibitors and squalene cyclase inhibitors, however, these assays are recognized by those skilled in the art. Thus, inhibition by combined squalene epoxidase/squalene cyclase inhibitors is readily determined by those skilled in art according to the aforementioned standard assays for squalene cyclase or squalene epoxidase inhibitors. A variety of these compounds are described and referenced below, however other squalene epoxidase/squalene cyclase inhibitors will be known to those skilled in the art, U.S. Pat. Nos. 5,084,461 and 5,278,171 (the disclosures of which are incorporated by reference) disclose certain azadecalin derivatives. EP publication 468,434 (the disclosure of which is incorporated by reference) discloses certain piperidyl ether and thio-ether derivatives such as 2-(1-piperidyl)pentyl isopentyl sulfoxide and 2-(1-piperidyl)ethyl ethyl sulfide. PCT publication WO 9401404 (the disclosure of which is hereby incorporated by reference) discloses certain acyl-piperidines such as 1-(1-oxopentyl-5-phenylthio)-4-(2-hydroxy-1-methyl)-ethyl)piperidine. U.S. Pat. No. 5,102,915 (the disclosure of which is hereby incorporated by reference) discloses certain cyclopropyloxy-squalene derivatives.

The compounds of the present invention may also be administered in combination with naturally occurring compounds that act to lower plasma cholesterol levels. These naturally occurring compounds are commonly called nutraceuticals and include, for example, garlic extract and niacin. A slow-release form of niacin is available and is known as Niaspan. Niacin may also be combined with other therapeutic agents such as lovastatin, or another is an HMG-CoA reductase inhibitor. This combination therapy with lovastatin is known as ADVICOR™ (Kos Pharmaceuticals Inc.).

Any cholesterol absorption inhibitor can be used as an additional in the combination aspect of the present invention. The term cholesterol absorption inhibition refers to the ability of a compound to prevent cholesterol contained within the lumen of the intestine from entering into the intestinal cells and/or passing from within the intestinal cells into the lymph system and/or into the blood stream. Such cholesterol absorption inhibition activity is readily determined by those skilled in the art according to standard assays (e.g., J. Lipid Res. (1993) 34, 377-395). Cholesterol absorption inhibitors are known to those skilled in the art and are described, for example, in PCT WO 94/00480. An example of a recently approved cholesterol absorption inhibitor is ZETIA™ (ezetimibe) (Schering-Plough/Merck).

Any ACAT inhibitor may be used in the combination therapy aspect of the present invention. The term ACAT inhibitor refers to compounds that inhibit the intracellular esterification of dietary cholesterol by the enzyme acyl CoA: cholesterol acyltransferase. Such inhibition may be determined readily by one of skill in the art according to standard assays, such as the method of Heider et al. described in Journal of Lipid Research., 24: 1127 (1983). A variety of these compounds are known to those skilled in the art, for example, U.S. Pat. No. 5,510,379 discloses certain carboxysulfonates, while WO 96/26948 and WO 96/10559 both disclose urea derivatives having ACAT inhibitory activity. Examples of ACAT inhibitors include compounds such as Avasimibe (Pfizer), CS-505 (Sankyo) and Eflucimibe (Eli Lilly and Pierre Fabre).

A lipase inhibitor may be used in the combination therapy aspect of the present invention. A lipase inhibitor is a compound that inhibits the metabolic cleavage of dietary triglycerides or plasma phospholipids into free fatty acids and the corresponding glycerides (e.g. EL, H, etc.). Under normal physiological conditions, lipolysis occurs via a two-step process that involves acylation of an activated serine moiety of the lipase enzyme. This leads to the production of a fatty acid-lipase hemiacetal intermediate, which is then cleaved to release a diglyceride. Following further deacylation, the lipase-fatty acid intermediate is cleaved, resulting in free lipase, a glyceride and fatty acid. In the intestine, the resultant free fatty acids and monoglycerides are incorporated into bile acid-phospholipid mircelles, which are subsequently absorbed at the level of the brush border of the small intestine. The micelles eventually enter the peripheral circulation as chylomicrons. Such lipase inhibition activity is readily determined by those skilled in the art according to standard assays (e.g., Methods Enzymol, 286: 190-231).

Pancreatic lipase mediates the metabolic cleavage of fatty acids from triglycerides at the 1- and 3-carbon positions. The primary site of the metabolism of ingested fats is in the duodenum and proximal jejunum by pancreatic lipase, which is usually secreted in vast excess of the amounts necessary for the breakdown of fats in the upper small intestine. Because pancreatic lipase is the primary enzyme required for the absorption of dietary triglycerides, inhibitors have utility in the treatment of obesity and the other related conditions. Such pancreatic lipase inhibition activity is readily determined by those skilled in the art according to standard assays (e.g., Methods Enzymol. 286: 190-231).

Gastric lipase is an immunologically distinct lipase that is responsible for approximately 10 to 40% of the digestion of dietary fats. Gastric lipase is secreted in response to mechanical stimulation, ingestion of food, the presence of a fatty meal or by sympathetic agents. Gastric lipolysis of ingested fats is of physiological importance in the provision of fatty acids needed to trigger pancreatic lipase activity in the intestine and is also of importance for fat absorption in a variety of physiological and pathological conditions associated with pancreatic insufficiency. See, for example, C. K. Abrams, et al., Gastroenterology, 92, 125 (1987). Such gastric lipase inhibition activity is readily determined by those skilled in the art according to standard assays (e.g., Methods Enzymol. 286: 190-231).

A variety of gastric and/or pancreatic lipase inhibitors are known to one of ordinary skill in the art. Preferred lipase inhibitors are those inhibitors that are selected from the group consisting of lipstatin, tetrahydrolipstatin (orlistat), valilactone, esterastin, ebelactone A, and ebelactone B. The compound tetrahydrolipstatin is especially preferred. The lipase inhibitor, N-3-trifluoromethylphenyl-N′-3-chloro-4′-trifluoromethylphenylurea, and the various urea derivatives related thereto, are disclosed in U.S. Pat. No. 4,405,644. The lipase inhibitor, esteracin, is disclosed in U.S. Pat. Nos. 4,189,438 and 4,242,453. The lipase inhibitor, cyclo-O,O′-[(1,6-hexanediyl)-bis-(iminocarbonyl)]dioxime, and the various bis(iminocarbonyl)dioximes related thereto may be prepared as described in Petersen et al., Liebig's Annalen, 582, 205-229 (1949).

A variety of pancreatic lipase inhibitors are described herein below. The pancreatic lipase inhibitors lipstatin, (2S,3S,5S,7Z,10Z)-5-[(S)-2-formamido-4-methyl-valeryloxy]-2-hexyl-3-hydroxy-7,10-hexadecanoic acid lactone, and tetrahydrolipstatin (orlistat), (2S,3S,5S)-5-[(S)-2-formamido-4-methyl-valeryloxy]-2-hexyl-3-hydroxy-hexadecanoic 1,3 acid lactone, and the variously substituted N-formylleucine derivatives and stereoisomers thereof, are disclosed in U.S. Pat. No. 4,598,089. For example, tetrahydrolipstatin is prepared as described in, e.g., U.S. Pat. Nos. 5,274,143, 5,420,305, 5,540,917, and 5,643,874. The pancreatic lipase inhibitor, FL-386, 1-[4-(2-methylpropyl)cyclohexyl-2-[(phenylsulfonyl)oxy]-ethanone, and the variously substituted sulfonate derivatives related thereto, are disclosed in U.S. Pat. No. 4,452,813. The pancreatic lipase inhibitor, WAY-121898, 4-phenoxyphenyl-4-methylpiperidin-1-yl-carboxylate, and the various carbamate esters and pharmaceutically acceptable salts related thereto, are disclosed in U.S. Pat. Nos. 5,512,565; 5,391,571 and 5,602,151. The pancreatic lipase inhibitor, valilactone, and a process for the preparation thereof by the milcrobial cultivation of Actinomycetes strain MG147-CF2, are disclosed in Kitahara et al., J. Antibiotics, 40 (11), 1647-1650 (1987). The pancreatic lipase inhibitors, ebelactone A and ebelactone B, and a process for the preparation thereof by the microbial cultivation of Actinomycetes strain MG7-G1, are disclosed in Umezawa, et al., J. Antibiotics, 33, 1594-1596 (1980). The use of ebelactones A and B in the suppression of monoglyceride formation is disclosed in Japanese Kokai 08-143457, published Jun. 4, 1996.

Other compounds that are marketed for hyperlipidemia, including hypercholesterolemia and which are intended to help prevent or treat atherosclerosis include bile acid sequestrants, such as Welchol®, Colestid®, LoCholest® and Questran®; and fibric acid derivatives, such as Atromid®, Lopid®, and Tricor®.

Diabetes can be treated by administering to a patient having diabetes (especially Type II), insulin resistance, impaired glucose tolerance, metabolic syndrome, or the like, or any of the diabetic complications such as neuropathy, nephropathy retinopathy or cataracts, a therapeutically effective amount of a compound of the present invention in combination with other agents (e.g., insulin) that can be used to treat diabetes. This includes the classes of anti-diabetic agents (and specific agents) described herein.

Any glycogen phosphorylase inhibitor can be used as the second agent in combination with a compound of the present invention. The term glycogen phosphorylase inhibitor refers to compounds that inhibit the bioconversion of glycogen to glucose-1-phosphate which is catalyzed by the enzyme glycogen phosphorylase. Such glycogen phosphorylase inhibition activity is readily determined by those skilled in the art according to standard assays e.g., J. Med. Chem. 41 (1998) 2934-2938). A variety of glycogen phosphorylase inhibitors are known to those skilled in the a including those described in WO 96/39384 and WO 96/39385.

Any aldose reductase inhibitor can be used in combination with a compound of the present invention. The term aldose reductase inhibitor refers to compounds that inhibit the bioconversion of glucose to sorbitol, which is catalyzed by the enzyme aldose reductase. Aldose reductase inhibition is readily determined by those skilled in the art according to standard assays (e.g. J. Malone, Diabetes, 29:861-864 (1980). “Red Cell Sorbitol, an Indicator of Diabetic Control”). A variety of aldose reductase inhibitors are known to those skilled in the art, such as those described in U.S. Pat. No. 6,579,879, which includes 6-(5-chloro-3-methyl-benzofuran-2-sulfonyl)-2H-pyridazin-3-one.

Any sorbitol dehydrogenase inhibitor can be used in combination with a compound of the present invention. The term sorbitol dehydrogenase inhibitor refers to compounds that inhibit the bioconversion of sorbitol to fructose which is catalyzed by the enzyme sorbitol dehydrogenase. Such sorbitol dehydrogenase inhibitor activity is readily determined by those skilled in the art according to standard assays (e.g., Analyt. Biochem (2000) 280: 329-331), A variety of sorbitol dehydrogenase inhibitors are known, for example U.S. Pat. Nos. 5,728,704 and 5,866,578 disclose compounds and a method for treating or preventing diabetic complications by inhibiting the enzyme sorbitol dehydrogenase.

Any glucosidase inhibitor can be used in combination with a compound of the present invention. A glucosidase inhibitor inhibits the enzymatic hydrolysis of complex carbohydrates by glycoside hydrolases, for example amylase or maltase, into bioavailable simple sugars, for example, glucose. The rapid metabolic action of glucosidases, particularly following the intake of high levels of carbohydrates, results in a state of alimentary hyperglycemia which, in adipose or diabetic subjects, leads to enhanced secretion of insulin, increased fat synthesis and a reduction in fat degradation. Following such hyperglycemias, hypoglycemia frequently occurs, due to the augmented levels of insulin present. Additionally, it is known chyme remaining in the stomach promotes the production of gastric juice, which initiates or favors the development of gastritis or duodenal ulcers. Accordingly, glucosidase inhibitors are known to have utility in accelerating the passage of carbohydrates through the stomach and inhibiting the absorption of glucose from the intestine. Furthermore, the conversion of carbohydrates into lipids of the fatty tissue and the subsequent incorporation of alimentary fat into fatty tissue deposits is accordingly reduced or delayed, with the concomitant benefit of reducing or preventing the deleterious abnormalities resulting therefrom. Such glucosidase inhibition activity is readily determined by those skilled in the art according to standard assays (e.g., Biochemistry (1969) 8: 4214).

A generally preferred glucosidase inhibitor includes an amylase inhibitor. An amylase inhibitor is a glucosidase inhibitor that inhibits the enzymatic degradation of starch or glycogen into maltose. Such amylase inhibition activity is readily determined by those skilled in the art according to standard assays (e.g., Methods Enzymol. (1955) 1: 149). The inhibition of such enzymatic degradation is beneficial in reducing amounts of bioavailable sugars, including glucose and maltose, and the concomitant deleterious conditions resulting therefrom.

A variety of glucosidase inhibitors are known to one of ordinary skill in the art and examples are provided below. Preferred glucosidase inhibitors are those inhibitors that are selected from the group consisting of acarbose, adiposine, voglibose, miglitol, emiglitate, camiglibose, tendamistate, trestatin, pradimicin-Q and salbostatin. The glucosidase inhibitor, acarbose, and the various amino sugar derivatives related thereto are disclosed in U.S. Pat. Nos. 4,062,950 and 4,174,439 respectively. The glucosidase inhibitor, adiposine, is disclosed in U.S. Pat. No. 4,254,256. The glucosidase inhibitor, voglibose, 3,4-dideoxy-4-[[2-hydroxy-1-(hydroxymethyl)ethyl]amino]-2-C-(hydroxymethyl)-D-epi-inositol, and the various N-substituted pseudo-aminosugars related thereto, are disclosed in U.S. Pat. No. 4,701,559. The glucosidase inhibitor, miglitol, (2R,3R,4R,5S)-1-(2-hydroxyethyl)-2-(hydroxymethyl)-3,4,5-piperidinetriol and the various 3,4,5-trihydroxypiperidines related thereto, are disclosed in U.S. Pat. No. 4,639,436. The glucosidase inhibitor, emiglitate, ethyl p-[2-[(2R,3R,4R,5S)-3,4,5-trihydroxy-2-hydroxymethyl)piperidino]ethoxy]-benzoate, the various derivatives related thereto and pharmaceutically acceptable acid addition salts thereof, are disclosed in U.S. Pat. No. 5,192,772. The glucosidase inhibitor, MDL-25637, 2,6-dideoxy-7-O-β-D-glucopyrano-syl-2,6-imino-D-glycero-L-gluco-heptitol, the various homodisaccharides related thereto and the pharmaceutically acceptable acid addition salts thereof, are disclosed in U.S. Pat. No. 4,634,765. The glucosidase inhibitor, camiglibose, methyl 6-deoxy-6-[(2R,3R,4R,5S)-3,4,5-trihydroxy-2-(hydroxymethyl)piperidino]-α-D-glucopyranoside sesquihydrate, the deoxy-nojirimycin derivatives related thereto, the various pharmaceutically acceptable salts thereof and synthetic methods for the preparation thereof, are disclosed in U.S. Pat. Nos. 5,157,116 and 5,504,078. The glycosidase inhibitor, salbostatin and the various pseudosaccharides related thereto, are disclosed in U.S. Pat. No. 5,091,524.

A variety of amylase inhibitors are known to one of ordinary skill in the art. The amylase inhibitor, tendamistat and the various cyclic peptides related thereto, are disclosed in U.S. Pat. No. 4,451,455. The amylase inhibitor Al-3688 and the various cyclic polypeptides related thereto are disclosed in U.S. Pat. No. 4,623,714. The amylase inhibitor, trestatin, consisting of a mixture of trestatin A, trestatin B and trestatin C and the various trehalose-containing aminosugars related thereto are disclosed in U.S. Pat. No. 4,273,765.

Additional anti-diabetic compounds, which can be used as the second agent in combination with a compound of the present invention, include, for example, the following: biguanides (e.g., metformin), insulin secretagogues (e.g., sulfonylureas and glinides), glitazones, non-glitazone PPARγ agonists, PPARβ agonists, inhibitors of DPP-IV, inhibitors of PDE5, inhibitors of GSK-3, glucagon antagonists, inhibitors of f-1,6-BPase(Metabasis/Sankyo), GLP-1/analogs (AC 2993, also known as exendin-4), insulin and insulin mimetics (Merck natural products). Other examples would include PKC-β inhibitors and AGE breakers.

The compounds of the present invention can be used in combination with anti-obesity agents. Any anti-obesity agent can be used as the second agent in such combinations and examples are provided herein. Such anti-obesity activity is readily determined by those skilled in the art according to standard assays known in the art.

Suitable anti-obesity agents include phenylpropanolamine, ephedrine, pseudoephedrine, phentermine, β₂ adrenergic receptor agonists, apolipoprotein-B secretion/microsomal triglyceride transfer protein (apo-B/MTP) inhibitors, MCR-4 agonists, cholecystokinin-A (CCK-A) agonists, monoamine reuptake inhibitors (e.g., sibutramine), sympathomimetic agents, serotoninergic agents, cannabinoid receptor (CB-1) antagonists (e.g., rimonabant described in U.S. Pat. No. 5,624,941 (SR-141,716A), purine compounds, such as those described in US Patent Publication No. 2004/0092520; pyrazolo[1,5-a][1,3,5]triazine compounds, such as those described in U.S. Non-Provisional patent application Ser. No. 10/763105 filed on Jan. 21, 2004; and bicyclic pyrazolyl and imidazolyl compounds, such as those described in U.S. Provisional Application No. 60/518280 filed on Nov. 7, 2003), dopamine agonists (e.g., bromocriptine), melanocyte-stimulating hormone receptor analogs, 5HT2c agonists, melanin concentrating hormone antagonists, leptin (the OB protein), leptin analogs, leptin receptor agonists, galanin antagonists, lipase inhibitors (e.g., tetrahydrolipstatin, i.e. orlistat), bombesin agonists, anorectic agents (e.g., a bombesin agonist), Neuropeptide-Y antagonists, thyroxine, thyromimetic agents, dehydroepiandrosterones or analogs thereof, glucocorticoid receptor agonists or antagonists, orexin receptor antagonists, urocortin binding protein antagonists, glucagon-like peptide-1 receptor agonists, ciliary neurotrophic factors (e.g., Axokine™), human agouti-related proteins (AGRP), ghrelin receptor antagonists, histamine 3 receptor antagonists or inverse agonists, neuromedin U receptor agonists, and the like.

Rimonabant (SR141716A also known under the tradename Acomplia™ available from Sanofi-Synthelabo) can be prepared as described in U.S. Pat. No. 5,624,941. Other suitable CB-1 antagonists include those described in U.S. Pat. Nos. 5,747,594, 6,432,984 and 6,518,264; U.S. Patent Publication Nos. US2004/0092520, US2004/0157839, US2004/0214855, and US2004/0214838; U.S. patent application Ser. No. 10/971,599 filed on Oct. 22, 2004; and PCT Patent Publication Nos. WO 02/076949, WO 03/075650, WO04/048317, WO04/013120, and WO 04/012671.

Preferred apolipoprotein-B secretion/microsomal triglyceride transfer protein (apo-B/MTP) inhibitors for use as anti-obesity agents are gut-selective MTP inhibitors, such as dirlotapide described in U.S. Pat. No. 6,720,351; 4-(4-(4-(4-((2-((4-methyl-4H-1,2,4-triazol-3-ylthio)methyl)-2-(4-chlorophenyl)-1,3-dioxolan-4-yl)methoxy)phenyl)piperazin-1-yl)phenyl)-2-sec-butyl-2H-1,2,4-triazol-3(4H)-one (R103757) described in U.S. Pat. Nos. 5,521,186 and 5,929,075; and implitapide (BAY 13-9952) described in U.S. Pat. No. 6,265,431. As used herein, the term “gut-selective” means that the MTP inhibitor has a higher exposure to the gastro-intestinal tissues versus systemic exposure.

Any thyromimetic can be used as the second agent in combination with a compound of the present invention. Such thyromimetic activity is readily determined by those skilled in the art according to standard assays (e.g., Atherosclerosis (1996) 126: 53-63). A variety of thyromimetic agents are known to those skilled in the art, for example those disclosed in U.S. Pat. Nos. 4,766,121, 4,826,876; 4,910,305; 5,061,796; 5,284,971; 5,401,772; 5,654,468; and 5,569,674. Other antiobesity agents include sibutramine which can be prepared as described in U.S. Pat. No. 4,929,629 and bromocriptine which can be prepared as described in U.S. Pat. Nos. 3,752,814 and 3,752,888.

The compounds of the present invention can also be used in combination with other antihypertensive agents. Any anti-hypertensive agent can be used as the second agent in such combinations and examples are provided herein. Such antihypertensive activity is readily determined by those skilled in the art according to standard assays (e.g., blood pressure measurements).

Examples of presently marketed products containing antihypertensive agents include calcium channel blockers, such as Cardizem®, Adalat®, Calan®, Cardene®, Covera®, Dilacor®, DynaCirc®, Procardia XL®, Sular®, Tiazac®, Vascor®, Verelan®, Isoptin®, Nimotop®, Norvasc®, and Plendil®; angiotensin converting enzyme (ACE) inhibitors, such as Accupril®, Altace®, Captopril®, Lotensin®, Mavik®, Monopril®, Prinivil®, Univasc®, Vasotec® and Zestril®.

Amlodipine and related dihydropyridine compounds are disclosed in U.S. Pat. No. 4,572,909, which is incorporated herein by reference, as potent anti-ischemic and antihypertensive agents. U.S. Pat. No. 4,879,303, which is incorporated herein by reference, discloses amlodipine benzenesulfonate salt (also termed amlodipine besylate). Amlodipine and amlodipine besylate are potent and long lasting calcium channel blockers. As such, amlodipine, amlodipine besylate, amlodipine maleate and other pharmaceutically acceptable acid addition salts of amlodipine have utility as antihypertensive agents and as antiischemic agents. Amlodipine besylate is currently sold as Norvasc®. Amlodipine has the formula

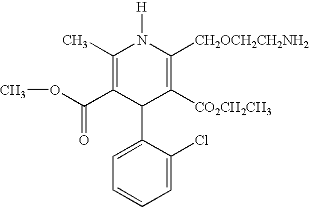

Calcium channel blockers which are within the scope of this invention include, but are not limited to: bepridil, which may be prepared as disclosed in U.S. Pat. No. 3,962,238 or U.S. Reissue No. 30,577; clentiazem, which may be prepared as disclosed in U.S. Pat. No. 4,567,175; diltiazem, which may be prepared as disclosed in U.S. Pat. No. 3,562: fendiline, which may be prepared as disclosed in U.S. Pat. No. 3,262,977; gallopamil, which may be prepared as disclosed in U.S. Pat. No. 3,261,859; mibefradil, which may be prepared as disclosed in U.S. Pat. No. 4,808,605; prenylamine, which may be prepared as disclosed in U.S. Pat. No. 3,152,173; semotiadil, which may be prepared as disclosed in U.S. Pat. No. 4,786,635; terodiline, which may be prepared as disclosed in U.S. Pat. No. 3,371,014; verapamil, which may be prepared as disclosed in U.S. Pat. No. 3,261,859; aranipine, which may be prepared as disclosed in U.S. Pat. No. 4,572,909; barnidipine, which may be prepared as disclosed in U.S. Pat. No. 4,220,649; benidipine, which may be prepared as disclosed in European Patent Application Publication No. 106,275; cilnidipine, which may be prepared as disclosed in U.S. Pat. No. 4,672,068; efonidipine, which may be prepared as disclosed in U.S. Pat. No. 4,685,264; elgodipine, which may be prepared as disclosed in U.S. Pat. No. 4,952,592; felodipine, which may be prepared as disclosed in U.S. Pat. No. 4,264,611; isradipine, which may be prepared as disclosed in U.S. Pat. No. 4,465,972; lacidipine, which may be prepared as disclosed in U.S. Pat. No. 4,801,599; lercanidipine, which may be prepared as disclosed in U.S. Pat. No. 4,705,797; manidipine, which may be prepared as disclosed in U.S. Pat. No. 4,892,875, nicardipine, which may be prepared as disclosed in U.S. Pat. No. 3,985,7586 nifedipine, which may be prepared as disclosed in U.S. Pat. No. 3,465,847; nilvadipine, which may be prepared as disclosed in U.S. Pat. No. 4,338,322, nimodipine, which may be prepared as disclosed in U.S. Pat. No. 3,799,934; nisoldipine, which may be prepared as disclosed in U.S. Pat. No. 4,154,839; nitrendipine, which may be prepared as disclosed in U.S. Pat. No. 3,799,934, cinnarizine, which may be prepared as disclosed in U.S. Pat. No. 2,882,271; flunarzine, which may be prepared as disclosed in U.S. Pat. No. 3,773,939; lidoflazine, which may be prepared as disclosed in U.S. Pat. No. 3,267,104; lomerizine, which may be prepared as disclosed in U.S. Pat. No. 4,663,325: bencyclane, which may be prepared as disclosed in Hungarian Patent No. 151,865: etafenone, which may be prepared as disclosed in German Patent No. 1,265,758; and perhexiline, which may be prepared as disclosed in British Patent No. 1,025,578. The disclosures of all such U.S. Patents are incorporated herein by reference

Angiotensin Converting Enzyme Inhibitors (ACE-Inhibitors) which are within the scope of this invention include, but are not limited to: alacepril, which may be prepared as disclosed in U.S. Pat. No. 4,248,883, benazepril, which may be prepared as disclosed in U.S. Pat. No. 4,410,520: captopril, which may be prepared as disclosed in U.S. Pat. Nos. 4,046,889 and 4,105,776: ceronapril, which may be prepared as disclosed in U.S. Pat. No. 4,452,790; delapril, which may be prepared as disclosed in U.S. Pat. No. 4,385,051; enalapril, which may be prepared as disclosed in U.S. Pat. No. 4,374,829; fosinopril, which may be prepared as disclosed in U.S. Pat. No. 4,337,201; imadapril, which may be prepared as disclosed in U.S. Pat. No. 4,508,727, lisinopril, which may be prepared as disclosed in U.S. Pat. No. 4,555,502; moveltopril, which may be prepared as disclosed in Belgian Patent No. 893,553; perindopril, which may be prepared as disclosed in U.S. Pat. No. 4,506,729; quinapril, which may be prepared as disclosed in U.S. Pat. No. 4,344,949; ramipril, which may be prepared as disclosed in U.S. Pat. No. 4,587,258; spirapril, which may be prepared as disclosed in U.S. Pat. No. 4,470,972; temocapril, which may be prepared as disclosed in U.S. Pat. No. 4,699,905; and trandolapril, which may be prepared as disclosed in U.S. Pat. No. 4,933,361. The disclosures of all such U.S. patents are incorporated herein by reference.

Angiotensin-II receptor antagonists (A-II antagonists) which are within the scope of this invention include, but are not limited to: candesartan, which may be prepared as disclosed in U.S. Pat. No. 5,196,444; eprosartan, which may be prepared as disclosed in U.S. Pat. No. 5,185,351; irbesartan, which may be prepared as disclosed in U.S. Pat. No. 5,270,317, losartan, which may he prepared as disclosed in U.S. Pat. No. 5,138,069; and valsartan, which may be prepared as disclosed in U.S. Pat. No. 5,399,578. The disclosures of all such U.S. patents are incorporated herein by reference.

Beta-adrenergic receptor blockers (beta or β-blockers) which are within the scope of this invention include, but are not limited to: acebutolol, which may be prepared as disclosed in U.S. Pat. No. 3,857,952; alprenolol, which may be prepared as disclosed in Netherlands Patent Application No. 6,605,692; amosulalol, which may be prepared as disclosed in U.S. Pat. No. 4,217,305; arotinolol, which may be prepared as disclosed in U.S. Pat. No. 3,932,400; atenolol, which may be prepared as disclosed in U.S. Pat. Nos. 3,663,607 or 3,836,671; befunolol, which may be prepared as disclosed in U.S. Pat. No. 3,853,923; betaxolol, which may be prepared as disclosed in U.S. Pat. No. 4,252,984; bevantolol, which may be prepared as disclosed in U.S. Pat. No. 3,857,981; bisoprolol, which may be prepared as disclosed in U.S. Pat. No. 4,171,370; bopindolol, which may be prepared as disclosed in U.S. Pat. No. 4,340,541; bucumolol, which may be prepared as disclosed in U.S. Pat. No. 3,663,570; bufetolol, which may be prepared as disclosed in U.S. Pat. No. 3,723,476; bufuralol, which may be prepared as disclosed in U.S. Pat. No. 3,929,836; bunitrolol, which may be prepared as disclosed in U.S. Pat. Nos. 3,940,489 and 3,961,071, buprandolol, which may be prepared as disclosed in U.S. Pat. No. 3,309,406, butiridine hydrochloride, which may be prepared as disclosed in French Patent No. 1,390,056; butofilolol, which may be prepared as disclosed in U.S. Pat. No. 4,252,825, carazolol, which may be prepared as disclosed in German Patent No 2,240,599; carteolol, which may be prepared as disclosed in U.S. Pat. No. 3,910,924; carvedilol, which may be prepared as disclosed in U.S. Pat. No. 4,503,067; celiprolol, which may be prepared as disclosed in U.S. Pat. No. 4,034,009; cetamolol, which may be prepared as disclosed in U.S. Pat. No. 4,059,622; coranolol, which may be prepared as disclosed in German Patent No. 2,213,044; dilevalol, which may be prepared as disclosed in Clifton et al., Journal of Medicinal Chemistry, 1982, 25, 670; epanolol, which may be prepared as disclosed in European Patent Publication Application No. 41,491; indenolol, which may be prepared as disclosed in U.S. Pat. No. 4,045,482, labetalol, which may be prepared as disclosed in U.S. Pat. No. 4,012,444; levobunolol, which may be prepared as disclosed in U.S. Pat. No. 4,463,176, mepindolol which may be prepared as disclosed in Seeman et al., Helv. Chim. Acta, 1971, 54, 241; metipranolol, which may be prepared as disclosed in Czechoslovakian Patent Application No. 128,471, metoprolol, which may be prepared as disclosed in U.S. Pat. No. 3,873,600; moprolol, which may be prepared as disclosed in U.S. Pat. No. 3,501,769I; nadolol, which may be prepared as disclosed in U.S. Pat. No. 3,935,267; nadoxolol, which may be prepared as disclosed in U.S. Pat. No. 3,819,702; nebivalol, which may be prepared as disclosed in U.S. Pat. No. 4,654,262; nipradilol, which may be prepared as disclosed in U.S. Pat. No. 4,394,382; oxprenolol, which may be prepared as disclosed in British Patent No. 1,077,603; perbutolol, which may be prepared as disclosed in U.S. Pat. No. 3,551,493; pindolol, which may be prepared as disclosed in Swiss Patent Nos. 469,002 and 472,404; practolol, which may be prepared as disclosed in U.S. Pat. No. 3,408,387; pronethalol, which may be prepared as disclosed in British Patent No. 909,367; propranolol, which may be prepared as disclosed in U.S. Pat. Nos. 3,337,628 and 3,520,919, sotalol, which may be prepared as disclosed in Uloth et al., Journal of Medicinal Chemistry, 1966, 9, 88; sufinalol, which may be prepared as disclosed in German Patent No. 2,728,641; talindol, which may be prepared as disclosed in U.S. Pat. Nos. 3,935,259 and 4,038,313: tertatolol, which may be prepared as disclosed in U.S. Pat. No. 3,960,891; tilisolol, which may be prepared as disclosed in U.S. Pat. No. 4,129,585; timolol, which may be prepared as disclosed in U.S. Pat. No. 3,655,663; toliprolol, which may be prepared as disclosed in U.S. Pat. No. 3,432,545; and xibenolol, which may be prepared as disclosed in U.S. Pat. No. 4,018,824. The disclosures of all such U.S. patents are incorporated herein by reference.

Alpha-adrenergic receptor blockers (alpha- or α-blockers) which are within the scope of this invention include, but are not limited to; amosulalol, which may be prepared as disclosed in U.S. Pat. No. 4,217,307; arotinolol, which may be prepared as disclosed in U.S. Pat. No. 3,932,400; dapiprazole, which may be prepared as disclosed in U.S. Pat. No. 4,252,721; doxazosin, which may be prepared as disclosed in U.S. Pat. No. 4,188,390; fenspiride, which may be prepared as disclosed in U.S. Pat. No. 3,399,192; indoramin, which may be prepared as disclosed in U.S. Pat. No. 3,527,761; labetolol, naftopidil, which may be prepared as disclosed in U.S. Pat. No. 3,997,666; nicergoline, which may be prepared as disclosed in U.S. Pat. No. 3,228,943; prazosin, which may be prepared as disclosed in U.S. Pat. No. 3,511,836; tamsulosin, which may be prepared as disclosed in U.S. Pat. No. 4,703,063; tolazoline, which may be prepared as disclosed in U.S. Pat. No. 2,161,938; trimazosin, which may be prepared as disclosed in U.S. Pat. No. 3,669,968, and yohimbine, which may be isolated from natural sources according to methods well known to those skilled in the art. The disclosures of all such U.S. patents are incorporated herein by reference.

The term “vasodilator,” where used herein, is meant to include cerebral vasadilators, coronary vasodilators and peripheral vasodilators. Cerebral vasodilators within the scope of this invention include, but are not limited to: bencyclane; cinnarizine; citicoline, which may be isolated from natural sources as disclosed in Kennedy et al., Journal of the American Chemical Society, 1955, 77, 250 or synthesized as disclosed in Kennedy, Journal of Biological Chemistry, 1956, 222, 185; cyclandelate, which may be prepared as disclosed in U.S. Pat. No. 3,663,597; ciclonicate, which may be prepared as disclosed in German Patent No. 1,910,481, diisopropylamine dichloroacetate, which may be prepared as disclosed in British Patent No. 862,248, eburnamonine, which may be prepared as disclosed in Hermann et al., Journal of the American Chemical Society, 1979, 101, 1540; fasudil, which may be prepared as disclosed in U.S. Pat. No. 4,678,783, fenoxedil, which may be prepared as disclosed in U.S. Pat. No. 3,818,021; flunarizine, which may be prepared as disclosed in U.S. Pat. No. 3,773,939; ibudilast, which may be prepared as disclosed in U.S. Pat. No. 3,850,941; ifenprodil, which may be prepared as disclosed in U.S. Pat. No. 3,509,164; lomerizine, which may be prepared as disclosed in U.S. Pat. No. 4,663,325; nafronyl, which may be prepared as disclosed in U.S. Pat. No. 3,334,096; nicametate, which may be prepared as disclosed in Blicke et al., Journal of the American Chemical Society, 1942, 64, 1722; nicergoline, which may be prepared as disclosed above, nimodipine, which may be prepared as disclosed in U.S. Pat. No. 3,799,934; papaverine, which may be prepared as reviewed in Goldberg, Chem. Prod. Chem. News, 1954, 17, 371; pentifylline, which may be prepared as disclosed in German Patent No. 860,217; tinofedrine, which may be prepared as disclosed in U.S. Pat. No. 3,563,997; vincamine, which may be prepared as disclosed in U.S. Pat. No. 3,770,724; vinpocetine, which may be prepared as disclosed in U.S. Pat. No. 4,035,750; and viquidil, which may be prepared as disclosed in U.S. Pat. No. 2,500,444. The disclosures of all such U.S. patents are incorporated herein by reference.

Coronary vasodilators within the scope of this invention include, but are not limited to: amotriphene, which may be prepared as disclosed in U.S. Pat. No. 3,010,965; bendazol, which may be prepared as disclosed in J. Chem. Soc. 1958, 2426; benfurodil hemisuccinate, which may be prepared as disclosed in U.S. Pat. No. 3,355,463; benziodarone, which may be prepared as disclosed in U.S. Pat. No. 3,012,042; chloracizine, which may be prepared as disclosed in British Patent No. 740,932; chromonar, which may be prepared as disclosed in U.S. Pat. No. 3,282,938; clobenfural, which may be prepared as disclosed in British Patent No. 1,160,925, clonitrate, which may be prepared from propanediol according to methods well known to those skilled in the art, e.g., see Annalen, 1870, 155, 165; cloricromen, which may be prepared as disclosed in U.S. Pat. No. 4,452,811; dilazep, which may be prepared as disclosed in U.S. Pat. No. 3,532,685; dipyridamole, which may be prepared as disclosed in British Patent No. 807,826; droprenilamine, which may be prepared as disclosed in German Patent No. 2,521,113; efloxate, which may be prepared as disclosed in British Patent Nos 803,372 and 824,547; erythrityl tetranitrate, which may be prepared by nitration of erythritol according to methods well-known to those skilled in the art; etafenone, which may be prepared as disclosed in German Patent No. 1,265,758; fendiline, which may be prepared as disclosed in U.S. Pat. No. 3,282,977; floredil, which may be prepared as disclosed in German Patent No. 2,020,464; ganglefene, which may be prepared as disclosed in U.S.S.R. Patent No. 115,905; hexestrol, which may be prepared as disclosed in U.S. Pat. No. 2,357,985; hexobendine, which may be prepared as disclosed in U.S. Pat. No. 3,267,103; itramin tosylate, which may be prepared as disclosed in Swedish Patent No. 168,308, khellin, which may be prepared as disclosed in Baxter et al., Journal of the Chemical Society, 1949, S 30; lidoflazine, which may be prepared as disclosed in U.S. Pat. No. 3,267,104; mannitol hexanitrate, which may be prepared by the nitration of mannitol according to methods well-known to those skilled in the art; medibazine, which may be prepared as disclosed in U.S. Pat. No. 3,119,826; nitroglycerin; pentaerythritol tetranitrate, which may be prepared by the nitration of pentaerythritol according to methods well-known to those skilled in the art; pentrinitrol, which may be prepared as disclosed in German Patent No. 638,422-3; perhexilline, which may be prepared as disclosed above; pimefylline, which may be prepared as disclosed in U.S. Pat. No. 3,350,400: prenylamine, which may be prepared as disclosed in U.S. Pat. No. 3,152,173; propatyl nitrate, which may be prepared as disclosed in French Patent No. 1,103,113; trapidil, which may be prepared as disclosed in East German Patent No. 55,956; tricromyl, which may be prepared as disclosed in U.S. Pat. No. 2,769,015; trimetazidine, which may be prepared as disclosed in U.S. Pat. No. 3,262,852; troinitrate phosphate, which may be prepared by nitration of triethanolamine followed by precipitation with phosphoric acid according to methods well-known to those skilled in the art; visnadine, which may be prepared as disclosed in U.S. Pat. Nos. 2,816,118 and 2,980,699. The disclosures of all such U.S. patents are incorporated herein by reference.

Peripheral vasodilators within the scope of this invention include, but are not limited to: aluminum nicotinate, which may be prepared as disclosed in U.S. Pat. No. 2,970,082; bamethan, which may be prepared as disclosed in Corrigan et al., Journal of the American Chemical Society, 1945, 67, 1894; bencyclane, which may be prepared as disclosed above; betahistine, which may be prepared as disclosed in Walter et al.; Journal of the American Chemical Society, 1941, 63, 2771, bradykinin, which may be prepared as disclosed in Hamburg et al., Arch. Biochem. Biophys., 1958, 76, 252; brovincamine, which may be prepared as disclosed in U.S. Pat. No. 4,146,643; bufeniode, which may be prepared as disclosed in U.S. Pat. No. 3,542,870; buflomedil, which may be prepared as disclosed in U.S. Pat. No. 3,895,030; butalamine, which may be prepared as disclosed in U.S. Pat. No. 3,338,899; cetiedil, which may be prepared as disclosed in French Patent Nos. 1,460,571; ciclonicate, which may be prepared as disclosed in German Patent No. 1,910,481; cinepazide, which may be prepared as disclosed in Belgian Patent No. 730,345; cinnarizine, which may be prepared as disclosed above; cyclandelate, which may be prepared as disclosed above; diisopropylamine dichloroacetate, which may be prepared as disclosed above; eledoisin, which may be prepared as disclosed in British Patent No, 984,810; fenoxedil, which may be prepared as disclosed above, flunarizine, which may be prepared as disclosed above; hepronicate, which may be prepared as disclosed in U.S. Pat. No. 3,384,642, ifenprodil, which may be prepared as disclosed above; iloprost, which may be prepared as disclosed in U.S. Pat. No. 4,692,464; inositol niacinate, which may be prepared as disclosed in Badgett et al., Journal of the American Chemical Society, 1947, 69, 2907; isoxsuprine, which may be prepared as disclosed in U.S. Pat. No. 3,056,836; kallidin, which may be prepared as disclosed in Biochem. Biophys. Res. Commun., 1961, 6, 210; kallikrein, which may be prepared as disclosed in German Patent No. 1,102,973; moxisylyte, which may be prepared as disclosed in German Patent No. 905,738; nafronyl, which may be prepared as disclosed above; nicametate, which may be prepared as disclosed above, nicergoline, which may be prepared as disclosed above; nicofuranose, which may be prepared as disclosed in Swiss Patent No. 366, 523; nylidrin, which may be prepared as disclosed in U.S. Pat. Nos. 2,661,372 and 2,661,373, pentifylline, which may be prepared as disclosed above; pentoxifylline, which may be prepared as disclosed in U.S. Pat. No. 3,422,107; piribedil, which may be prepared as disclosed in U.S. Pat. No. 3,299,067; prostaglandin E₁, which may be prepared by any of the methods referenced in the Merck Index, Twelfth Edition, Budaveri, Ed., New Jersey, 1996, p. 1353; suloctidil, which may be prepared as disclosed in German Patent No. 2,334,404; tolazoline, which may be prepared as disclosed in U.S. Pat. No. 2,161,938; and xanthinol niacinate, which may be prepared as disclosed in German Patent No. 1,102,750 or Korbonits et al. Acta. Pharm. Hung., 1968, 38, 98. The disclosures of all such U.S. patents are incorporated herein by reference.

The term “diuretic,” within the scope of this invention, is meant to include diuretic benzothiadiazine derivatives, diuretic organomercurials, diuretic purines, diuretic steroids, diuretic sulfonamide derivatives, diuretic uracils and other diuretics such as amanozine, which may be prepared as disclosed in Austrian Patent No, 168,063, amiloride, which may be prepared as disclosed in Belgian Patent No. 639,386; arbutin, which may be prepared as disclosed in Tschitschibabin, Annalen, 1930, 479, 303; chlorazanil, which may be prepared as disclosed in Austrian Patent No. 168,063; ethacrynic acid, which may be prepared as disclosed in U.S. Pat. No. 3,255,241; etozolin, which may be prepared as disclosed in U.S. Pat. No. 3,072,653; hydracarbazine, which may be prepared as disclosed in British Patent No. 856,409; isosorbide, which may be prepared as disclosed in U.S. Pat. No. 3,160,641; mannitol; metochalcone, which may be prepared as disclosed in Freudenberg et al., Ber., 1957, 90, 957; muzolimine, which may be prepared as disclosed in U.S. Pat. No. 4,018,890; perhexiline, which may be prepared as disclosed above; ticrynafen, which may be prepared as disclosed in U.S. Pat. No. 3,758,506, triamterene which may be prepared as disclosed in U.S. Pat. No. 3,081,230; and urea. The disclosures of all such U.S. patents are incorporated herein by reference.

Diuretic benzothiadiazine derivatives within the scope of this invention include, but are not limited to: althiazide, which may be prepared as disclosed in British Patent No. 902,658; bendroflumethiazide, which may be prepared as disclosed in U.S. Pat. No. 3,265,573; benzthiazide, McManus et al, 136th Am. Soc. Meeting (Atlantic City, September 1959). Abstract of papers, pp 13-O; benzylhydrochlorothiazide, which may be prepared as disclosed in U.S. Pat. No. 3,108,097; buthiazide, which may be prepared as disclosed in British Patent Nos. 861,367 and 885,078; chlorothiazide, which may be prepared as disclosed in U.S. Pat. Nos. 2,809,194 and 2,937,169; chlorthalidone, which may be prepared as disclosed in U.S. Pat. No. 3,055,904; cyclopenthiazide, which may be prepared as disclosed in Belgian Patent No. 587,225, cyclothiazide, which may be prepared as disclosed in Whitehead et al., Journal of Organic Chemistry. 1961, 26, 2814; epithiazide, which may be prepared as disclosed in U.S. Pat. No. 3,009,911; ethiazide, which may be prepared as disclosed in British Patent No. 861,367; fenquizone, which may be prepared as disclosed in U.S. Pat. No. 3,870,720; indapamide, which may be prepared as disclosed in U.S. Pat. No. 3,565,911; hydrochlorothiazide, which may be prepared as disclosed in U.S. Pat. No. 3,164,588; hydroflumethiazide, which may be prepared as disclosed in U.S. Pat. No. 3,254,076; methyclothiazide, which may be prepared as disclosed in Close et al., Journal of the American Chemical Society. 1960, 82, 1132; meticrane, which may be prepared as disclosed in French Patent Nos. M2790 and 1,365,504; metolazone, which may be prepared as disclosed in U.S. Pat. No. 3,360,518; paraflutizide, which may be prepared as disclosed in Belgian Patent No. 620,829; polythiazide, which may be prepared as disclosed in U.S. Pat. No. 3,009,911; quinethazone, which may be prepared as disclosed in U.S. Pat. No. 2,976,289; teclothiazide, which may be prepared as disclosed in Close et al., Journal of the American Chemical Society, 1960, 82, 1132, and trichlormethiazide, which may be prepared as dislcosed in deStevens et al., Experientia, 1960, 16, 113. The disclosures of all such U.S. patents are incorporated herein by reference.

Diuretic sulfonamide derivatives within the scope of this invention include, but are not limited to: acetazolamide, which may be prepared as disclosed in U.S. Pat. No. 2,980,679; ambuside, which may be prepared as disclosed in U.S. Pat. No. 3,188,329; azosemide, which may be prepared as disclosed in U.S. Pat. No. 3,665,002; bumetanide, which may be prepared as disclosed in U.S. Pat. No. 3,634,583; butazolamide, which may be prepared as disclosed in British Patent No. 769,757; chloraminophenamide, which may be prepared as disclosed in U.S. Pat. Nos. 2,809,194, 2,965,655 and 2,965,656; clofenamide, which may be prepared as disclosed in Olivier, Rec. Trav. Chim. 1918, 37, 307; clopamide, which may be prepared as disclosed in U.S. Pat. No. 3,459,756; clorexolone, which may be prepared as disclosed in U.S. Pat. No. 3,183,243; disulfamide, which may be prepared as disclosed in British Patent No. 851,287; ethoxolamide, which may be prepared as disclosed in British Patent No. 795,174, furosemide, which may be prepared as disclosed in U.S. Pat. No. 3,058,882; mefruside, which may be prepared as disclosed in U.S. Pat. No. 3,356,692; methazolamide, which may be prepared as disclosed in U.S. Pat. No. 2,783,241; piretanide, which may be prepared as disclosed in U.S. Pat. No. 4,010,273; torasemide, which may be prepared as disclosed in U.S. Pat. No. 4,018,929; tripamide, which may be prepared as disclosed in Japanese Patent No. 73 05,585; and xipamide, which may be prepared as disclosed in U.S. Pat. No. 3,567,777. The disclosures of all such U.S. patents are incorporated herein by reference.

Osteoporosis is a systemic skeletal disease, characterized by low bone mass and deterioration of bone tissue, with a consequent increase in bone fragility and susceptibility to fracture. In the U.S., the condition affects more than 25 million people and causes more than 1.3 million fractures each year, including 500,000 spine, 250,000 hip and 240,000 wrist fractures annually. Hip fractures are the most serious consequence of osteoporosis, with 5-20% of patients dying within one year, and over 50% of survivors being incapacitated.

The elderly are at greatest risk of osteoporosis, and the problem is therefore predicted to increase significantly with the aging of the population. Worldwide fracture incidence is forecasted to increase three-fold over the next 60 years, and one study has estimated that there will be 4.5 million hip fractures worldwide in 2050.

Women are at greater risk of osteoporosis than men. Women experience a sharp acceleration of bone loss during the five years following menopause. Other factors that increase the risk include stoking, alcohol abuse, a sedentary lifestyle and low calcium intake.

Those skilled in the art will recognize that anti-resorptive agents (for example progestins, polyphosphonates, bisphosphonate(s), estrogen agonists/antagonists, estrogen, estrogen/progestin combinations, Premarin®, estrone, estriol or 17α- or 17β-ethynyl estradiol) may be used in conjunction with the compounds of the present invention.

Exemplary progestins are available from commercial sources and include: algestone acetophenide, altrenogest, amadinone acetate, anagestone acetate, chlormadinone acetate, cingestol, clogestone acetate, clomegestone acetate, delmadinone acetate, desogestrel, dimethisterone, dydrogesterone, ethynerone, ethynodiol diacetate, etonogestrel, flurogestone acetate, gestaclone, gestodene, gestonorone caproate, gestrinone, haloprogesterone, hydroxyprogesterone caproate, levonorgestrel, lynestrenol, medrogestone, medroxyprogesterone acetate, melengestrol acetate, methynodiol diacetate, norethindrone, norethindrone acetate, norethynodrel, norestimate, norgestomet, norgestrel, oxogestone phenpropionate, progesterone, quingestanol acetate, quingestrone, and tigestol.

Preferred progestins are medroxyprogestrone, norethindrone and norethynodrel.

Exemplary bone resorption inhibiting polyphosphonates include polyphrosphonates of the type disclosed in U.S. Pat. No. 3,683,080, the disclosure of which is incorporated herein by reference. Preferred polyphosphonates are geminal diphosphonates (also referred to as bis-phosphonates). Tiludronate disodium is an especially preferred polyphosphonate. Ibandronic acid is an especially preferred polyphosphonate. Alendronate and resindronate are especially preferred polyphosphonates. Zoledronic acid is an especially preferred polyphosphonate. Other preferred polyphosphonates are 6-amino-1-hydroxy-hexylidene-bisphosphonic acid and 1-hydroxy-3(methylpentylamino)-propylidene-bisphosphonic acid. The polyphosphonates may be administered in the form of the acid, or of a soluble alkali metal salt or alkaline earth metal salt. Hydrolyzable esters of the polyphosphonates are likewise included. Specific examples include ethane-1-hydroxy 1,1-diphosphonic acid, methane diphosphonic acid, pentane-1-hydroxy-1,1-diphosphonic acid, methane dichloro diphosphonic acid, methane hydroxy diphosphonic acid, ethane-1-amino-1,1-diphosphonic acid, ethane-2-amino-1,1-diphosphonic acid, propane-3-amino-1-hydroxy-1,1-diphosphonic acid, propane-N,N-dimethyl-3-amino-1-hydroxy-1,1-diphosphonic acid, propane-3,3-dimethyl -3-amino-1-hydroxy-1,1-diphosphonic acid, phenyl amino methane diphosphonic acid, N,N-dimethytamino methane diphosphonic acid, N(2-hydroxyethyl)amino methane diphosphonic acid, butane-4-amino-1-hydroxy-1,1-diphosphonic acid, pentane-5-amino-1-hydroxy-1,1-diphosphonic acid, hexane-6-amino-1-hydroxy-1,1-diphosphonic acid and pharmaceutically acceptable esters and salts thereof.

In particular, the compounds of this invention may be combined with a mammalian estrogen agonist/antagonist. Any estrogen agonist/antagonist may be used in the combination aspect of this invention. The term estrogen agonist/antagonist refers to compounds which bind with the estrogen receptor, inhibit bone turnover and/or prevent bone loss. In particular, estrogen agonists are herein defined as chemical compounds capable of binding to the estrogen receptor sites in mammalian tissue, and mimicking the actions of estrogen in one or more tissue. Estrogen antagonists are herein defined as chemical compounds capable of binding to the estrogen receptor sites in mammalian tissue, and blocking the actions of estrogen in one or more tissues. Such activities are readily determined by those skilled in the art of standard assays including estrogen receptor binding assays, standard bone histomorphometric and densitometer methods, and Eriksen E. F. et al., Bone Histomorphometry, Raven Press, New York, 1994, pages 1-74, Grier S. J. et. al., The Use of Dual-Energy X-Ray Absorptiometry In Animals, Inv, Radiol., 1996, 31(1):50-62; Wahner H. W. and Fogelman I., The Evaluation of Osteoporosis: Dual Energy X-Ray Absorptiometry in Clinical Practice., Martin Dunitz Ltd., London 1994, pages 1-296). A variety of these compounds are described and referenced below.

Another preferred estrogen agonist/antagonist is 3-{4-(1,2-diphenyl-but-1-enyl}-phenyl)-acrylic acid, which is disclosed in Willson et al., Endocrinology, 1997, 138, 3901-3911.

Another preferred estrogen agonist/antagonist is tamoxifen: (ethanamine,2-(4-(1,2-diphenyl-1-butenyl)phenoxy)-N,N-dimethyl, (Z)-2-, 2-hydroxy-1,2,3-propanetricarboxylate(1:1)) and related compounds which are disclosed in U.S. Pat. No. 4,536,516, the disclosure of which is incorporated herein by reference.

Another related compound is 4-hydroxy tamoxifen, which is disclosed in U.S. Pat. No. 4,623,660, the disclosure of which is incorporated herein by reference.

A preferred estrogen agonist/antagonist is raloxifene: (methhanone, (6-hydroxy-2-(4-hydroxyphenyl)benzo[b]thien-3-yl)(4-(2-(1-piperdinyl)ethoxy)phenyl)-hydrochloride) which is disclosed in U.S. Pat. No. 4,418,068, the disclosure of which is incorporated herein by reference.

Another preferred estrogen agonist/antagonist is toremifene: (ethanamine, 2-(4-(4-chloro-1,2-diphenyl-1-butenyl)phenoxy)-N,N-dimethyl-(Z)-, 2-hydroxy-1,2,3-propanetricarboxylate (1:1) which is disclosed in U.S. Pat. No. 4,996,226, the disclosure of which is incorporated herein by reference.

Another preferred estrogen agonist/antagonist is centchroman: 1-(2-((4-(-methoxy-2,2, dimethyl-3-phenyl-chroman-4-yl)-phenoxy)-ethyl)-pyrrolidine, which is disclosed in U.S. Pat. No. 3,822,287, the disclosure of which is incorporated herein by reference. Also preferred is levormeloxifene.

Another preferred estrogen agonist/antagonist is idoxifene: (E)-1-(2-(4-(1-(4-iodo-phenyl)-2-phenyl-but-1-enyl)phenoxy)ethyl)-pyrrolidine, which is disclosed in U.S. Pat. No. 4,839,155, the disclosure of which is incorporated herein by reference.

Another preferred estrogen agonist/antagonist is 2-(4-methoxy-phenyl)-3-[4-(2-piperidin-1-yl-ethoxy)-phenoxy]-benzo[b]thiophen-6-ol which is disclosed in U.S. Pat. No. 5,488,058, the disclosure of which is incorporated herein by reference.

Another preferred estrogen agonist/antagonist is 6-(4-hydroxy-phenyl)-5-(4-(2-piperidin-1-yl-ethoxy)-benzyl)-naphthalen-2-ol, which is disclosed in U.S. Pat. No. 5,484,795, the disclosure of which is incorporated herein by reference.

Another preferred estrogen agonist/antagonist is (4-(2-(2-aza-bicyclo[2.2.1]hept-2-yl)-ethoxy)-phenyl)-(6-hydroxy-2-(4-hydroxy-phenyl)-benzo[b]thiophen-3-yl)-methanone which is disclosed, along with methods of preparation, in PCT publication no. WO 95/10513 assigned to Pfizer Inc.

Other preferred estrogen agonist/antagonists include the compounds, TSE-424 (Wyeth-Ayerst Laboratories) and arazoxifene.

Other preferred estrogen agonist/antagonists include compounds as described in commonly assigned U.S. Pat. No. 5,552,412, the disclosure of which is incorporated herein by reference. Especially preferred compounds described therein are:
cis-6-(4-fluoro-phenyl)-5-(4-(2-piperidin-1-ethoxy)-phenyl)-5,6,7,8-tetrahydro-naphthalene-2-ol;
(−)-cis-6-phenyl-5-(4-(2-pyrrolidin-1-yl-ethoxy)-phenyl)-5,6,7,8-tetrahydro-naphthalene-2-ol (also known as lasofoxifene);
cis-6-phenyl-5-(4-(2-pyrrolidin-1-yl-ethoxy)-phenyl)-5,6,7,8-tetrahydro-napthalene-2-ol;
cis-1-(6′-pyrrolodinoethioxy-3′-pyridyl)-2-phenyl-6-hydroxy-1,2,3,4-tetrahydro-naphthalene;
1-(4′-pyrrolidinoethoxyphenyl)-2-(4″-fluorophenyl)-6-hydroxy-1,2,3,4-tetrahydroisoquinoline;
cis-6-(4-hydroxyphenyl)-5-(4-(2-piperidin-1-yl-ethoxy)-phenyl)-5,6,7,8-tetrahydro-naphthalene-2-ol; and
1-(4′-pyrrolidinolethoxyphenyl)-2-(4″-fluorophenyl-6-hydroxy-1,2,3,4-tetrahydroisoquinoline.

Other estrogen agonist/antagonists are described in U.S. Pat. No. 4,133,814 (the disclosure of which is incorporated herein by reference). U.S. Pat. No. 4,133,814 discloses derivatives of 2-phenyl-3-aroyl-benzothiophene and 2-phenyl-3-aroylbenzothiophene-1-oxide.

Other anti-osteoporosis agents, which can be used as the second agent in combination with a compound of the present invention, include, for example, the following parathyroid hormone (PTH) (a bone anabolic agent); parathyroid hormone (PTH) secretagogues (see, e.g., U.S. Pat. No. 6,132,774), particularly calcium receptor antagonists; calcitonin, and vitamin D and vitamin D analogs.

Any selective androgen receptor modulator (SARM) can be used in combination with a compound of the present invention. A selective androgen receptor modulator (SARM) is a compound that possesses androgenic activity and which exerts tissue-selective effects. SARM compounds can function as androgen receptor agonists, partial agonists, partial antagonists or antagonists. Examples of suitable SARMs include compounds such as cyproterone acetate, chlormadinone, flutamide, hydroxyflutamide, bicalutamide, nilutamide, spironolactone, 4-(trifluoromethyl)-2(1H)pyrrolidino[3,2-g]quinoline derivatives, 1,2-dihydropyridino[5,6-g]quinoline derivatives and piperidino[3,2-g]quinolinone derivatives.

Cypterone, also known as (1b,2b)-6-chloro-1,2-dihydro-17-hydroxy-3′H-cyclopropa[1,2]pregna-1,4,6-triene-3,20-dione is disclosed in U.S. Pat. No. 3,234,093. Chlormadinone, also known as 17-(acetyloxy)-6-chloropregna-4,6-diene-3,20-dione, in its acetate form, acts as an anti-androgen and is disclosed in U.S. Pat. No. 3,485,852. Nilutamide, also known as 5,5-dimethyl-3-[4-nito-3-(trifluoromethyl)phenyl]-2,4-imidazolidinedione and by the trade name Nilandron® is disclosed in U.S. Pat. No. 4,097,578. Flutamide, also known as 2-methyl-N-[4-nitro-3-(trifluoromethyl)phenyl]propanamide and the trade name Eulexin® is disclosed in U.S. Pat. No. 3,847,988. Bicalutamide, also known as 4′-cyano-a′, a′,a′-trifuoro-3-(4-fluorophenylsufonyl)-2-hydroxy-2-methylpropiono-m-toluidide and the trade name Casodex® is disclosed in EP-100172. The enantiomers of biclutamide are discussed by Tucker and Chesterton, J. Med. Chem. 1988, 31, 885-887. Hydroxyflutamide, a known androgen receptor antagonist in most tissues, has been suggested to function as a SARM for effects on IL-6 production by osteoblasts as disclosed in Hofbauer et al. J. Bone Miner. Res. 1999, 14, 1330-1337. Additional SARMs have been disclosed in U.S. Pat. No. 6,017,924; WO 01/16108, WO 01/16133: WO 01/16139, WO 02/00617, WO 02/16310, U.S. Patent Application Publication No. US 2002/0099096, U.S. Patent Application Publication No. US 2003/0022868, WO 03/011302 and WO 03/011824. All of the above refences are hereby incorporated by reference herein.

The starting materials and reagents for the above described compounds, are also readily available or can be easily synthesized by those skilled in the art using conventional methods of organic synthesis. For example, many of the compounds used herein, are related to, or are derived from compounds in which there is a large scientific interest and commercial need, and accordingly many such compounds are commercially available or are reported in the literature or are easily prepared from other commonly available substances by methods which are reported in the literature.

Some of the compounds of this invention or intermediates in their synthesis have asymmetric carbon atoms and therefore are enantiomers or diastereomers. Diasteromeric mixtures can be separated into their individual diastereomers on the basis of their physical chemical differences by methods known per se, for example, by chromatography and/or fractional crystallization. Enantiomers can be separated by, for example, chiral HPLC methods or converting the enantiomeric mixture into a diastereomeric mixture by reaction with an appropriate optically active compound (e.g., alcohol), separating the diastereomers and converting (e.g., hydrolyzing) the individual diastereomers to the corresponding pure enantiomers. Also, an enantiomeric mixture of the compounds or an intermediate in their synthesis which contain an acidic or basic moiety may be separated into their corresponding pure enantiomers by forming a diastereomic salt with an optically pure chiral base or acid (e.g., 1-phenyl-ethyl amine, dibenzyl tartrate or tartaric acid) and separating the diasteromers by fractional crystallization followed by neutralization to break the salt, thus providing the corresponding pure enantiomers. All such isomers, including diastereomers, enantiomers and mixtures thereof are considered as part of this invention for all of the compounds of the present invention, including the compounds of the present invention. Also, some of the compounds of this invention are atropisomers (e.g., substituted biaryls) and are considered as part of this invention.

More specifically, the compounds of this invention may be obtained in enantiomerically enriched form by resolving the racemate of the final compound or an intermediate in its synthesis, employing chromatography (preferably high pressure liquid chromatography [HPLC]) on an asymmetric resin (preferably Chiralcel™ AD or OD (obtained from Chiral Technologies, Exton, Pa.)) with a mobile phase consisting of a hydrocarbon (preferably heptane or hexane) containing between 0 and 5% isopropanol (preferably between 2 and 20%) and between 0 and 5% of an alkyl amine (preferably 0.1% of diethylamine). Concentration of the product containing fractions affords the desired materials.

Some of the compounds of this invention are acidic and they form a salt with a pharmaceutically acceptable cation. Some of the compounds of this invention are basic and they form a salt with a pharmaceutically acceptable anion. All such salts are within the scope of this invention and they can be prepared by conventional methods such as combining the acidic and basic entities, usually in a stoichiometric ratio, in either an aqueous, non-aqueous or partially aqueous medium, as appropriate. The salts are recovered either by filtration, by precipitation with a non-solvent followed by filtration, by evaporation of the solvent, or, in the case of aqueous solutions, by lyophilization, as appropriate. The compounds can be obtained in crystalline form by dissolution in an appropriate solvent(s) such as ethanol, hexanes or water/ethanol mixtures.

In addition, when the compounds of this invention form hydrates or solvates they are also within the scope of the invention.

The compounds of this invention, their prodrugs and the salts of such compounds and prod rugs are all adapted to therapeutic use as agents that inhibit cholesterol ester transfer protein activity in mammals, particularly humans. Thus, the compounds of this invention elevate plasma HDL cholesterol, its associated components, and the functions performed by them in mammals, particularly humans. By virtue of their activity, these agents also reduce plasma levels of triglycerides, VLDL cholesterol, Apo-B, LDL cholesterol and their associated components in mammals, particularly humans. Moreover, these compounds are useful in equaling LDL cholesterol and HDL cholesterol. Hence, these compounds are useful for the treatment and correction of the various dyslipidemias observed to be associated with the development and incidence of atherosclerosis and cardiovascular disease, including coronary artery disease, coronary heart disease, coronary vascular disease, peripheral vascular disease, hypoalphalipoproteinemia, hyperbetalipoproteinemia, hypetriglyceridemia, hyperoholesterolemia, familial-hypercholesterolemia, low HDL and associated components, elevated LDL and associated components, elevated Lp(a), elevated small-dense LDL, elevated VLDL and associated components and post-prandial lipemia.

Further, introduction of a functional CETP gene into an anima lacking CETP (mouse) results in reduced HDL levels (Agellon, L. B., et al: J. Biol. Chem. (1991) 266: 10796-10801.) and increased susceptibility to atherosclerosis. (Marotti, K. R., et al: Nature (1993) 364; 73-75.). Also, inhibition of CETP activity with an inhibitory antibody raises HDL-cholesterol in hamster (Evans, G. F., et al; J. of Lipid Research (1994) 35: 1634-1645.) and rabbit (Whitlock, M. E.; et al: J. Clin. Invest. (1989) 84: 129-137). Suppression of increased plasma CETP by intravenous injection with antisense oligodeoxynucleotides against CETP mRNA reduced atherosclerosis in cholesterol-fed rabbits (Sugano, M., et al: J. of Biol. Chem. (1998) 273: 5033-5036.) Importantly, human subjects deficient in plasma CETP, due to a genetic mutation possess markedly elevated plasma HDL-cholesterol levels and apolipoprotein A-I, the major apoprotein component of HDL. In addition, most demonstrate markedly decreased plasma LDL cholesterol and apolipoprotein B (the major apolipoprotein component of LDL. (Inazu, A., Brown, M. L., Hesler, C. B., et al.: N. Engl. J. Med. (1990) 323: 123-1238.)

Given the negative correlation between the levels of HDL cholesterol and HDL associated lipoproteins, and the positive correlation between triglycerides, LDL cholesterol, and their associated apolipoproteins in blood with the development of cardiovascular, cerebral vascular and peripheral vascular diseases, the compounds of this invention, their prod rugs and the salts of such compounds and prodrugs, by virtue of their pharmacologic action, are useful for the prevention, arrestment and/or regression of atherosclerosis and its associated disease states. These include cardiovascular disorders (e.g., angina, ischemia, cardiac ischemia and myocardial infarction), complications due to cardiovascular disease therapies (e.g., reperfusion injury and angioplastic restenosis), hypertension, elevated cardiovascular risk associated with hypertension, stroke, atherosclerosis associated with organ transplantation, cerebrovascular disease, cognitive dysfunction (including, but not limited to, dementia secondary to atherosclerosis, transient cerebral ischemic attacks, neurodegeneration, neuronal deficient, and delayed onset or procession of Alzheimer's disease), elevated levels of oxidative stress, elevated levels of C-Reactive Protein, Metabolic Syndrome and elevated levels of HbA1C.

Because of the beneficial effects widely associated with elevated HDL levels, an agent which inhibits CETP activity in humans, by virtue of its HDL increasing ability, also provides valuable avenues for therapy in a number of other disease areas as well.

Thus, given the ability of the compounds of this invention, their prodrugs and the salts of such compounds and prodrugs to alter lipoprotein composition via inhibition of cholesterol ester transfer, they are of use in the treatment of vascular complications associated with diabetes, lipoprotein abnormalities associated with diabetes and sexual dysfunction associated with diabetes and vascular disease. Hyperlipidemia is present in most subjects with diabetes mellitus (Howard, B. V. 1987. J. Lipid Res. 28, 613). Even in the presence of normal lipid levels, diabetic subjects experience a greater risk of cardiovascular disease (Kannel, W. B. and McGee, D. L. 1979. Diabetes Care 2, 120), CETP-mediated choesteryl ester transfer is known to be abnormally increased in both insulin-dependent (Bagdade, J. D., Subbaiah, P. V and Ritter, M. C. 1991. Eur. J. Clin. Invest. 21, 161) and non-insulin dependent diabetes (Bagdade. J. D., Ritter, M. C., Lane, J. and Subbaiah. 1993. Atherosclerosis 104, 691). It has been suggested that the abnormal increase in cholesterol transfer results in changes in lipoprotein composition, particularly for VLDL and LDL, that are more atherogenic (Bagdade, J. D., Wagner. J. D., Rudel, L. L., and Clarkson, T. B. 1995. J. Lipid Res. 36, 759). These changes would not necessarily be observed during routine lipid screening. Thus the present invention will be useful in reducing the risk of vascular complications as a result of the diabetic condition.

The described agents are useful in the treatment of obesity and elevated cardiovascular risk associated with obesity. In both humans (Radeau, T., Lau, P., Robb, M., McDonnell, M., Alihaud, G. and McPherson, R., 1995. Journal of Lipid Research. 36 (12):2552-61) and nonhuman primates (Quinet, E., Tall, A., Ramakrishnan, R., and Rudel, L., 1991. Journal of Clinical Investigation. 87 (5): 1559-66) mRNA for CETP is expressed at high levels in adipose tissue. The adipose message increases with fat feeding (Martin, L. J. Connelly, P. W., Nancoo, D., Wood, N., Zhang, Z. J., Maguire, G., Quinet, E., Tall, A. R., Marcel, Y. L. and McPherson, R., 1993. Journal of Lipid Research. 34 (3):437-46), and is translated into functional transfer protein and through secretion contributes significantly to plasma CETP levels in human adipocytes the bulk of cholesterol is provided by plasma LDL and HDL (Fong, B, S., and Angel, A. 1989. Biochimica et Biophysica Acta. 1004 (1,53-60). The uptakke of HDL cholesteryl ester is dependent in large part on CETP (Benoist, F., Lau, P., McDonnell, M., Doelle, H., Milne, R. and McPherson. R., 1997. Journal of Biological Chemistry. 272 (38):23572-7). This ability of CETP to stimulate HDL cholesteryl uptake, coupled with the enhanced binding of HDL to adipocytes in obese subjects (Jimenez, J. G., Fong, B., Julien, P. Despres, J. P., Rotstein, L., and Angel, A., 1989. International Journal of Obesity. 13 (5):699-709), suggests a role for CETP, not only in generating the low HDL phenotype for these subjects, but in the development of obesity itself by promoting cholesterol accumulation. Inhibitors of CETP activity that block this process therefore serve as useful adjuvants to dietary therapy in causing weight reduction.

CETP inhibitors are useful in the treatment of inflammation due to Gram-negative sepsis and septic shock. For example, the systemic toxicity of Gram-negative sepsis is in large part due to endotoxin, a lipopolysaccharide (LPS) released from the outer surface of the bacteria, which causes an extensive inflammatory response. Lipopolysaccharide can form complexes with lipoproteins (Ulevitch, R. J., Johnston, A. R., and Weinstein, D. B., 1981. J. Clin. Invest. 67, 827-37). In vitro studies have demonstrated that binding of LPS to HOL substantially reduces the production and release of mediators of inflammation (Ulevitch, R. J., Johhston, A. R. 1978. J. Clin. Invest, 62, 1313-24). In vivo studies show that transgenic mice expressing human apo-Al and elevated HDL levels are protected from septic shock (Levine, D. M., Parker, T. S., Donnelly, T. M., Walsh, A. M., and Rubin, A. L. 1993. Proc. Natl. Acad. Sci. 90, (12040-44). Importantly, administration of reconstituted HDL to humans challenged with endotoxin resulted in a decreased inflammatory response (Pajkrt, D., Doran. J. E., Koster, F., Lerch, P. G. Arnet. B., van der Poll, T., ten Cate, J. W. and van Deventer, S. J. H. 1996. J. Exp. Med. 184, 1601-08). The CETP inhibitors, by virtue of the fact that they raise HDL levels, attenuate the development of inflammation and septic shock.

The utility of the compounds of the invention, their prodrugs and the salts of such compounds and prodrugs as medical agents in the treatment of the above described disease/conditions in mammals (e.g. humans, male or female) is demonstrated by the activity of the compounds of this invention in conventional assays and the in vivo assay described below. The in vivo assay (with appropriate modifications within the skill in the art) may be used to determine the activity of other lipid or triglyceride controlling agents as well as the compounds of this invention. Such assays also provide a means whereby the activities of the compounds of this invention, their prodrugs and the salts of such compounds and prodrugs (or the other agents described herein) can be compared to each other and with the activities of other known compounds.

The results of these comparisons are useful for determining dosage levels in mammals, including humans, for the treatment of such diseases.

The following protocols may of course be varied by those skilled in the art.

The hyperalphacholesterolemic activity of the compounds may be determined by assessing the effect of these compounds on the action of cholesteryl ester transfer protein by measuring the relative transfer ratio of radiolabeled lipids between lipoprotein fractions, essentially as previously described by Morton in J. Biol. Chem. 256, 11992, 1981 and by Dias in Clin. Chem. 34, 2322, 1988.

### CETP in Vitro Assay

The following is a brief description of assays of cholesteryl ester transfer in 97% (whole) or diluted human plasma (in vitro) and animal plasma (ex vivo): CETP activity in the presence or absence of drug is assayed by determining the transfer of ³H-labeled cholesteryl oleate (CO) from exogenous tracer HDL or LDL to the nonHDL or HDL lipoprotein fraction in human plasma, respectvely, or from ³H-labeled LDL to the HDL fraction in animal plasma. Labeled human lipoprotein substrates are prepared similarly to the method described by Morton in which the endogenous CETP activity in plasma is employed to transfer ³H—CO from phospholipid liposomes to all the lipoprotein fractions in plasma. ³H-labeled LDL and HDL are subsequently isolated by sequential ultracentrifugation at the density cuts of 1.019-1.063 and 1.10-1.21 g/ml, respectively.

For the 97% or whole plasma activity assay, ³H-labeled HDL is added to plasma at 10-25 nmoles CO/ml and the samples incubated at 37° C. for 2.5-3 hrs. Non-HDL lipoproteins are then precipitated by the addition of an equal volume of 20% (wt/vol) polyethylene glycol 8000 (Dias). The samples are centrifuged 750 g×20 minutes and the radioactivity contained in the HDL-containing supernatant determined by liquid scintillation counting. Introducing varying quantities of the compounds of this invention as a solution in dimethylsulfoxide into human plasma, before addition of the radiolabeled cholesteryl oleate, and comparing the amounts of radiolabel transferred compared to incubations containing no inhibitor compounds allows the cholesteryl ester transfer inhibitory activities to be determined.

When a more sensitive assay is desirable, an in vitro assay using diluted human plasma is utilized. For this assay, ³H-labeled LDL is added to plasma at 50 nmoles CO/ml and the samples incubated at 37° C. for 7 hrs. Non-HDL lipoproteins are then precipitated by the addition of potassium phosphate to 100 mM final concentration followed by manganese chloride to 20 mM final concentration. After vortexing, the samples are centrifuged 750 g×20 minutes and the radioactivity contained in the HDL-containing supernatant determined by liquid scintillation counting. Introducing varying quantities of the compounds of this invention as a solution in dimethylsulfoxide into diluted human plasma, before addition of the radiolabeled cholesteryl oleate, and comparing the amounts of radiolabel transferred compared to incubations containing no inhibitor compounds allows the cholesteryl ester transfer inhibitory activities to be determined. This assay has been adapted to run in microtiter plate format with liquid scintillation counting accomplished using a Wallac plate reader.

### CETP In Vivo Assay

Activity of these compounds in vivo may be determined by the amount of agent required to be administered, relative to control, to inhibit cholesteryl ester transfer activity by 50% at various time points ex vivo or to elevate HDL cholesterol by a given percentage in a CETP-containing animal species. Transgenic mice expressing both human CETP and human apolipoprotein Al (Charles River, Boston, Mass.) may be used to assess compounds in vivo. The compounds to be examined are administered by oral gavage in an emulsion vehicle containing 20% (v:v) olive oil and 80% sodium taurocholate (0.5%). Blood is taken from mice retroorbitally before dosing, if a predose blood sample is desirable. At various times after dosing, ranging from 4 h to 24 h, the animals are sacrificed, blood obtained by heart puncture, and lipid parameters measured, including total cholesterol, HDL and LDL cholesterol, and triglycerides. CETP activity is determined by a method similar to that described above except that 5H-cholesteryl oleate-containing LDL is used as the donor source as opposed to HDL. The values obtained for lipids and transfer activity are compared to those obtained prior to dosing and/or to those from mice receiving vehicle alone.

### Plasma Lipids Assay

The activity of these compounds may also be demonstrated by determining the amount of agent required to alter plasma lipid levels, for example HDL cholesterol levels, LDL cholesterol levels, VLDL cholesterol levels or triglycerides, in the plasma of certain mammals, for example marmosets that possess CETP activity and a plasma lipoprotein profile similar to that of humans (Crook et al. Arteriosclerosis 10, 625, 1990). Adult marmosets are assigned to treatment groups so that each group has a similar mean±SD for total, HDL, and/or LDL plasma cholesterol concentrations. After group assignment, marmosets are dosed daily with compound as a dietary admix or by intragastric intubation for from one to eight days. Control marmosets receive only the dosing vehicle. Plasma total, LDL VLDL and HDL cholesterol values may be determined at any point during the study by obtaining blood from an antecubital vein and separating plasma lipoproteins into their individual subclasses by density gradient centrifugation, and by measuring cholesterol concentration as previously described (Crook et al. Arteriosclerosis 10, 625, 1990).

### In Vivo Atherosclerosis Assay

Anti-atherosclerotic effects of the compounds may be determined by the amount of compound required to reduce the lipid deposition in rabbit aorta. Male New Zealand White rabbits are fed a diet containing 0.2% cholesterol and 10% coconut oil for 4 days (meal-fed once per day). Rabbits are bled from the marginal ear vein and total plasma cholesterol values are determined from these samples. The rabbits are then assigned to treatment groups so that each group has a similar mean±SD for total plasma cholesterol concentration, HLDL cholesterol concentration, triglyceride concentration and/or cholesteryl ester transfer protein activity. After group assignment, rabbits are dosed daily with compound given as a dietary admix or on a small piece of gelatin based confection. Control rabbits receive only the dosing vehicle, be it the food or the gelatin confection. The cholesterol/coconut oil diet is continued along with the compound administration throughout the study. Plasma cholesterol values and cholesteryl ester transfer protein activity may be determined at any point during the study by obtaining blood from the marginal ear vein. After 3-5 months, the rabbits are sacrificed and the aortae are removed from the thoracic arch to the branch of the iliac arteries. The aortae are cleaned of adventitia, opened longitudinally and then analyzed unstained or stained with Sudan IV as described by Holman et. al. (Lab. Invest. 1958, 7, 42-47). The percent of the lesioned surface area is quantitated by densitometry using an Optimas Image Analyzing System (Image Processing Systems). Reduced lipid deposition is indicated by a reduction in the percent of lesioned surface area in the compound-receiving group in comparison with the control rabbits.

### Antiobesity Protocol

The ability of CETP inhibitors to cause weight loss may be assessed in obese human subjects with body mass index (BMI)≧30 kg/m². Doses of inhibitor are administered sufficient to result in an increase of ≧25% in HDL cholesterol levels. BMI and body fat distribution, defined as waist (W) to hip (H) ratio (WHR), are monitored during the course of the 3-6 month studies, and the results for treatment groups compared to those receiving placebo.

### In Vivo Sepsis Assay

In vivo studies show that transgenic mice expressing human apo-Al and elevated HDL levels are protected from septic shock. Thus the ability of CETP inhibitors to protect from septic shook may be demonstrated in transgenic mice expressing both human apo-Al and human CETP transgenes (Levine, D. M., Parker, T. S., Donnelly, T. M., Walsh, A. M. and Rubin, A. L., 1993. Proc. Natl. Acad. Sci. 90, 12040-44). LPS derived from E. coli is administered at 30 mg/kg by i.p. injection to animals which have been administered a CETP inhibitor at an appropriate dose to result in elevation of HDL. The number of surviving mice is determined at times up to 48 h after LPS injection and compared to those mice administered vehicle (minus CETP inhibitor) only.

### In Vivo Blood Pressure Assay

In Vivo Rabbit Model

Methods. New Zealand White male rabbits (3-4 kg) are anesthetized with sodium pentobarbital (30 mg/kg, i.v.) and a surgical plane of anesthesia is maintained by a continuous infusion of sodium pentobarbital (16 mg/kg/hr) via an ear vein catheter. A tracheotomy is performed through a ventral mindline cervical incision and the rabbits are ventilated with 100% oxygen using a positive pressure ventilator. Body temperature is maintained at 38.5° C. using a heating pad connected to a YSI temperature controller model 72 (Yellow Springs Instruments, Yellow Springs, Md.). Fluid-filled catheters are placed in the right jugular vein (for intravenous drug administration) and in the right carotid artery for arterial pressure monitoring and for blood gas analysis using a model 248 blood gas analyzer (Bayer Diagnostics, Norwood, Mass.). The ventilator is adjusted as needed to maintain blood pH and pCO₂ within normal physiological ranges for rabbits. Arterial pressure is measured using a strain gauge transducer (Spectromed, Oxnard, Calif.), previously calibrated using a mercury manometer, positioned at the level of the heart and connected to the arterial catheter. Arterial pressure signals are digitized at 500 Hz and analyzed using a Po-Ne-Mah Data Acquisition System (Gould Instrument Systems, Valley View, Ohio) to obtain mean arterial pressure and heart rate values. Baseline values are collected when mean arterial pressure and heart rate have stabilized. The test compound is then administered either as a subcutaneous (SC) bolus or as an intravenous (IV) infusion. For subcutaneous (SC) dosing the test compound can be dissolved in an appropriate vehicle such as 5% ethanol in water (5% EtOH:95% H₂O), while for intravenous dosing the test compound can be dissolved in an appropriate vehicle such as 0.9% normal saline. Arterial pressure and heart rate are monitored continuously for 4 hours following dosing of the test compound or for the duration of a continuous 4 hour infusion of the test compound. Blood is sampled after dosing or during the infusion of the test compound to determine plasma concentrations of the test compounds.

In Vivo Primate Model

Methods: Adult M. fascicularis primates (6-8 kg) that have been previously instrumented with subcutaneous vascular access ports in the descending thoracic aorta and conditioned to sit quietly in specially designed primate-restraining chairs are used. All primates are fasted for 12-18 hours prior to the experiment. On the day of the experiment, with the primates restrained in the chairs, a strain gauge pressure transducer (Spectromed, Oxnard, Calif.), previously calibrated using a mercury manometer, is positioned at the level of the heart and connected to the vascular access port to measure arterial pressure. The primates are allowed to acclimate to the chair for at least one hour. Arterial pressure signals are digitized at 500 Hz and continuously recorded throughout the experiment and analyzed using a Po-Ne-NMah Data Acquisition System (Gould Instrument Systems, Valley View, Ohio) to obtain the measurements of mean arterial pressure and heart rate. Baseline values are collected when the primates are sitting calmly and when mean arterial pressure and heart rate have stabilized. The test compound is then administered as a subcutaneous (SC) bolus of a solution of the test compound in an appropriate vehicle such as 5% ethanol in water (5% EtOH:95% H₂O). The solution of test compound or vehicle is filtered through a 0.22 micron filter prior to injection and a typical dosing volume is 0.2 ml/kg. Arterial pressure and heart rate are monitored continuously for 4 hours following dosing of the test compound and are recorded at selected time intervals for data comparison (vehicle vs test compound). Blood samples (1.5 ml) are withdrawn to determine plasma concentrations of the test compound and withdrawn blood is immediately replaced with 0.9% sterile saline to maintain blood volume.

Administration of the compounds of this invention may be via any method which delivers a compound of this invention systemically and/or locally. These methods include oral routes, parenteral, intraduodenal routes, etc. Generally, the compounds of this invention are administered orally, but parenteral administration (e.g., intravenous, intramuscular, subcutaneous or intramedullary) may be utilized, for example, where oral administration is inappropriate for the target or where the patient is unable to ingest the drug.

In general an amount of a compound of this invention is used that is sufficient to achieve the therapeutic effect desired (e.g., HDL elevation).

In general an effective dosage for the compounds of this invention is about 0.001 to 100 mg/kg/day of the compound, a prodrug thereof, or a pharmaceutically acceptable salt of said compound or of said prodrug. An especially preferred dosage is about 0.01 to 10 mg/kg/day of the compound, a prodrug thereof, or a pharmaceutically acceptable salt of said compound or of said prodrug.

A dosage of the combination pharmaceutical agents to be used in conjuction with the CETP inhibitors is used that is effective for the indication being treated.

For example, typically an effective dosage for HMG-CoA reductase inhibitors is in the range of 0.01 to 100 mg/kg/day. In general an effect dosage for a PPAR modulator is in the range of 0.01 to 100 mg/kg/day.

The compounds of the present invention are generally administered in the form of a pharmaceutical composition comprising at least one of the compounds of this invention together with a pharmaceutically acceptable vehicle, diluent or carrier as described below. Thus, the compounds of this invention may be administered individually or together in any conventional oral, parenteral, rectal or transdermal dosage form.

For oral administration a pharmaceutical composition may take the form of solutions, suspensions, tablets, pills, capsules, powders, and the like. Tablets containing various excipients such as sodium citrate, calcium carbonate and calcium phosphate are employed along with various disintegrants such as starch and preferably potato or tapioca starch and certain complex silicates, together with binding agents such as polyvinylpyrrolidone, sucrose, gelatin and acacia. Additionally, lubricating agents such as magnesium stearate, sodium lauryl sulfate and talc are often very useful for tabletting purposes. Solid compositions of a similar type are also employed as filters in soft and hard-filled gelatin capsules; preferred materials in this connection also include lactose or milk sugar as well as high molecular weight polyethylene glycols. A preferred formulation is a solution or suspension in an oil, for example, a vegetable oil, such as olive oil; triglycerides such as those marketed under the name, Miglyol™, or mono- or diglycerides such as those marketed under the name, Capmul™, for example, in a soft gelatin capsule. Antioxidants may be added to prevent long-term degradation as appropriate. When aqueous suspensions and/or elixirs are desired for oral administration, the compounds of this invention can be combined with various sweetening agents, flavoring agents, coloring agents, emulsifying agents and/or suspending agents, as well as such diluents as water, ethanol, propylene glycol, glycerin and various like combinations thereof.

Pharmaceutical compositions comprising a solid amorphous dispersion of a cholesteryl ester transfer protein (CETP) inhibitor and a concentration-enhancing polymer are described in International Publication No. WO 02/11710, which is hereby incorporated by reference herein. Self-emulsifying formulations of cholesteryl ester transfer protein (CETP) inhibitors are described in International Publication No. WO 03/000295, which is hereby incorporated by reference herein. Methods for depositing small drug crystals on excipients are set forth in the literature, such as in J. Pharm. Pharmacol. 1987, 39:769-773, which is hereby incorporated by reference herein. Moreover, the present invention includes formulations of a CETP inhibitor and a high surface area substrate, wherein the CETP inhibitor and substrate are combined to form an adsorbate.

Solid amorphous dispersions, including dispersions formed by a spray-drying process, are also a preferred dosage form for the poorly soluble compounds of the invention. By “solid amorphous dispersion” is meant a solid material in which at least a portion of the poorly soluble compound is in the amorphous form and dispersed in a polymer, By “amorphous” is meant that the poorly soluble compound is not crystalline. By “crystalline” is meant that the compound exhibits long-range order in three dimensions of at least 100 repeat units in each dimension. Thus, the term amorphous is intended to include not only material which has essentially no order, but also material which may have some small degree of order, but the order is in less than three dimensions and/or is only over short distances. Amorphous material may be characterized by techniques known in the art such as powder x-ray diffraction (PXRD) crystallography, solid state NMR, or thermal techniques such as differential scanning calorimetry (DSC). At least a major portion (i.e., at least about 60 wt %) of the poorly soluble compound in the solid amorphous dispersion is amorphous. Preferably, at least 75 wt % of the drug and more preferably at least 90 wt % of the drug in the solid amorphous dispersion is amorphous.

The compound can exist within the solid amorphous dispersion in relatively pure amorphous domains or regions, as a solid solution of the compound homogeneously distributed throughout the polymer or any combination of these states or those states that lie intermediate between them. Preferably, at least a portion of the drug and polymer are present as a solid solution. Preferably, the solid amorphous dispersion is substantially homogeneous so that the amorphous compound is dispersed as homogeneously as possible throughout the polymer. As used herein, “substantially homogeneous” means that the fraction of the compound that is present in relatively pure amorphous domains or regions within the solid amorphous dispersion is relatively small, on the order of less than 20 wt %, and preferably less than 10 wt % of the total amount of drug. Such substantially homogeneous solid amorphous dispersions are sometimes referred to in the art as solid solutions or molecular dispersions.

Polymers suitable for use in the solid amorphous dispersions should be inert, in the sense that they do not chemically react with the poorly soluble compound in an adverse manner, are pharmaceutically acceptable, and have at least some solubility in aqueous solution at physiologically relevant pHs (e.g. 1-8). The polymer can be neutral or ionizable, and should have an aqueous-solubility of at least 0.1 mg/mL over at least a portion of the pH range of 1-8.

Polymers suitable for use with the present invention may be cellulosic or non-cellulosic. The polymers may be neutral or ionizable in aqueous solution. Of these, ionizable and cellulosic polymers are preferred, with ionizable cellulosic polymers being more preferred.

Exemplary polymers include hydroxypropyl methyl cellulose acetate succinate (HPMCAS), hydroxypropyl methyl cellulose (HPMC), hydroxypropyl methyl cellulose phthalate (HPMCP), carboxy methyl ethyl cellulose (CMEC), cellulose acetate phthalate (CAP), cellulose acetate trimellitate (CAT), polyvinylpyrrolidone (PVP), hydroxypropyl cellulose (HPC), methyl cellulose (MC), block copolymers of ethylene oxide and propylene oxide (PEO/PPO, also known as poloxamers), and mixtures thereof. Especially preferred polymers include HPMCAS, HPMC, HPMCP, CMEC, CAP, CAT, PVP, poloxamers, and mixtures thereof. Most preferred is HPMCAS. See US Published Patent Application Publication No. 2002/0009494, the disclosure of which is incorporated herein by reference.

The solid amorphous dispersions may be prepared according to any process for forming solid amorphous dispersions that results in at least a major portion (at least 60%) of the poorly soluble compound being in the amorphous state. Such processes include mechanical, thermal and solvent processes. Exemplary mechanical processes include milling and extrusion; melt processes including high temperature fusion, solvent-modified fusion and melt-congeal processes; and solvent processes including non-solvent precipitation, spray coating and spray drying. See, for example, the following U.S. Patents, the pertinent disclosures of which are incorporated herein by reference: U.S. Pat. Nos. 5,456,923 and 5,939,099, which describe forming dispersions by extrusion processes; U.S. Pat. Nos. 5,340,591 and 4,673,564 which describe forming dispersions by milling processes; and U.S. Pat. Nos. 5,707,646 and 4,894,235, which describe forming dispersions by melt congeal processes. In a preferred process, the solid amorphous dispersion is formed by spray drying, as disclosed in US Patent Application Publication No 2005/0031692. In this process, the compound and polymer are dissolved in a solvent, such as acetone or methanol, and the solvent is then rapidly removed from the solution by spray drying to form the solid amorphous dispersion.

The solid amorphous dispersions are generally in the form of small particles. The particles are often less than 500 microns, and may be less than 200 microns, or even less than 100 microns.

The solid amorphous dispersions may be prepared to contain up to about 99 wt % of the compound, e.g., 1 wt %, 5 wt %, 10 wt %, 25 wt %, 50 wt %, 75 wt %, 95 wt %, or 98 wt % of the compound as desired. In general, solid amorphous dispersions having from 5 wt % to 75 wt % of the compound are preferred, and from 10 wt % to 50 wt % are more preferred.

The solid amorphous dispersion particles consist of mostly drug and polymer, with optional additives such as surfactants in minor amounts. The drug and polymer collectively constitute at least 50 wt % of the solid amorphous dispersion, and may constitute at least 60 wt %, at least 75 wt %, or even at least 90 wt % of the solid amorphous dispersion. In one embodiment, the solid amorphous dispersion consists essentially of the drug and polymer.

In another embodiment, the dosage form comprises an adsorbate of amorphous compound adsorbed onto a high surface area substrate. At least a major portion (i.e., at least about 60 wt %) of the poorly soluble compound in the solid amorphous dispersion is amorphous. Preferably, at least 75 wt % of the drug and more preferably at least 90 wt % of the drug in the solid amorphous dispersion is amorphous.

The adsorbate also includes a high surface area substrate. The substrate may be any material that is inert, meaning that the substrate does not adversely interact with the drug to an unacceptably high degree and which is pharmaceutically acceptable. The substrate also has a high surface area, meaning that the substrate has a surface area of at least 20 m²/g, preferably at least 50 m²/g, more preferably at least 100 m²/g, and most preferably at least 180 m²/g. The surface area of the substrate may be measured using standard procedures. One exemplary method is by low-temperature nitrogen adsorption, based on the Brunauer, Emmett, and Teller (BET) method, well known in the art. Thus, effective substrates can have surface areas of up to 200 m²/g, up to 400 m²/g and up to 600 m²/g or more. The substrate should also be in the form of small particles ranging in size of from 10 nm to 1 μm, preferably ranging in size from 20 nm to 100 nm. These particles may in turn form agglomerates ranging in size from 10 nm to 100 μm. The substrate is also insoluble in the process environment used to form the adsorbate. That is, where the adsorbate is formed by solvent processing, the substrate does not dissolve in the solvent. Where the adsorbate is formed by a melt or thermal process, the adsorbate has a sufficiently high melting point that it does not melt.

Exemplary materials which are suitable for the substrate include oxides, such as SiO₂, TiO₂, ZnO₂, ZnO, Al₂O₃, MgAlSilicate, calcium silicate (Zeodor™ and Zeopharm®), AlOH₂, magnesium oxide, magnesium trisilicate, silicon dioxide (Cab-O-Sil® or Aerosil®), zeolites, and other inorganic molecular sieves; inorganic materials such as silica, fumed silica (such as Aeroperl® and Aerosil® from Degussa, Parsippany, N.J.), dibasic calcium phosphate, calcium carbonate magnesium hydroxide, and talc; clays, such as kaolin (hydrated aluminum silicate), bentonite (hydrated aluminum silicate), hectorite and Veegum®; Na-, Al-, and Fe-montmorillonite; water insoluble polymers, such as cross-linked cellulose acetate phthalate, cross-linked hydroxypropyl methyl cellulose acetate succinate, cross-linked polyvinyl pyrrolidinone, (also known as cross povidone), microcrystalline cellulose, polyethylene/polyvinyl alcohol copolymer, polyethylene polyvinyl pyrrolidone copolymer, cross-linked carboxymethyl cellulose, sodium starch glycolate, cross-linked polystyrene divinyl benzene; and activated carbons, including those made by carbonization of polymers such as polyimides, polyacrylonitrile, phenolic resins, cellulose acetate, regenerated cellulose, and rayon. Highly porous materials such as calcium silicate and silicone dioxide are preferred.

In one embodiment, the adsorbate may further comprise a polymer. Polymers suitable for incorporation into the adsorbate include those suitable for use in a solid amorphous dispersion. A preferred polymer is polyvinylpyrrolidone.

The adsorbate may be prepared according to any process for forming adsorbates that results in at least a major portion (at least 60%) of the poorly soluble compound being in the amorphous state. Such processes include mechanical, thermal and solvent processes. Exemplary methods are disclosed in US Published Patent Application No. 2003/0054037.

The adsorbate may be prepared to contain up to about 99 wt % of the compound, e.g., 1 wt %, 5 wt %, 10 wt %, 25 wt %, 50 wt % 75 wt %, 95 wt %, or 98 wt % of the compound as desired. In general, adsorbates having from 5 wt % to 75 wt % of the compound are preferred, and from 10 wt % to 50 wt % are more preferred.

The adsorbates consist of mostly drug and substrate, with optional additives such as polymers described above or surfactants in minor amounts. The drug and substrate collectively constitute at least 50 wt % of the adsorbate, and may constitute at least 60 wt %, at least 75 wt %, or even at least 90% of the adsorbate. In one embodiment, the adsorbate consists essentially of the drug and substrate. For those embodiments including a polymer, the adsorbate may comprise up to 50 wt % polymer.

For purposes of parenteral administration, solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes. In this connection, the sterile aqueous media employed are all readily obtainable by standard techniques well-known to those skilled in the art.

For purposes of transdermal (e.g., topical) administration, dilute sterile, aqueous or partially aqueous solutions (usually in about 0.1% to 5% concentration), otherwise similar to the above parenteral solutions, are prepared.

Methods of preparing various pharmaceutical compositions with a certain amount of active ingredient are known, or will be apparent in light of this disclosure, to those skilled in this art. For examples of methods of preparing pharmaceutical compositions, see Remington's Pharmaceutical Sciences, Mack Publishing Company, Easter, Pa., 15th Edition (1975).

Pharmaceutical compositions according to the invention may contain 0.1%-95% of the compound(s) of this invention, preferably 1%-70%. In any event, the composition or formulation to be administered will contain a quantity of a compound(s) according to the invention in an amount effective to treat the disease/condition of the subject being treated, e.g., atherosclerosis.

Since the present invention has an aspect that relates to the treatment of the disease/conditions described herein with a combination of active ingredients which may be administered separately, the invention also relates to combining separate pharmaceutical compositions in kit form. The kit comprises two separate pharmaceutical compositions: a compound of the present invention, a prod rug thereof or a salt of such compound or prodrug and a second compound as described above. The kit comprises means for containing the separate compositions such as a container, a divided bottle or a divided foil packet. Typically the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules may be removed from the blister pack by manually applying pressure on the recesses whereby an opening is formed in the sheet at the place of the recess. The tablet or capsule may then be removed via said opening.

It may be desirable to provide a memory aid on the kit, e.g., in the form of numbers next to the tablets or capsules whereby the numbers correspond with the days of the regimen which the tablets or capsules so specified should be ingested. Another example of such a memory aid is a calendar printed on the card, e.g., as follows “First Week, Monday, Tuesday, . . . etc. Second Week, Monday, Tuesday, . . . ” etc. Other variations of memory aids will be readily apparent. A “daily dose” may be a single tablet or capsule or several pills or capsules to be taken on a given day. Also, a daily dose of compounds of the present invention may consist of one tablet or capsule while a daily dose of the second compound may consist of several tablets or capsules and vice versa. The memory aid should reflect this.

In another specific embodiment of the invention, a dispenser designed to dispense the daily doses one at a time in the order of their intended use is provided. Preferably, the dispenser is equipped with a memory-aid, so as to further facilitate compliance with the regimen. An example of such a memory-aid is a mechanical counter which indicates the number of daily doses that has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

The compounds of this invention either alone or in combination with each other or other compounds generally will be administered in a convenient formulation. The following formulation examples only are illustrative and are not intended to limit the scope of the present invention.

In the formulations which follow, “active ingredient” means a compound of this invention.

Formulation 1: Gelatin Capsules

Hard gelatin capsules are prepared using the following:

| Ingredient | Quantity (mg/capsule) |
|---|---|
| Active ingredient | 0.25-100 |
| Starch, NF | 0-650 |
| Starch flowable powder | 0-50 |
| Silicone fluid 350 centistokes | 0-15 |

A tablet formulation is prepared using the ingredients below.

Formulation 2: Tablet

| Ingredient | Quantity (mg/tablet) |
|---|---|
| Active ingredient | 0.25-100 |
| Cellulose, microcrystalline | 200-650 |
| Silicon dioxide, fumed | 10-650 |
| Stearate acid | 5-15 |

The components are blended and compressed to form tablets.

Alternatively, tablets each containing 0.25-100 mg of active ingredients are made up as follows:

Formulation 3: Tablets

| Ingredient | Quantity (mg/tablet) |
|---|---|
| Active ingredient | 0.25-100 |
| Starch | 45 |
| Cellulose, microcrystalline | 35 |
| Polyvinylpyrrolidone (as 10% solution in water) | 4 |
| Sodium carboxymethyl cellulose | 4.5 |
| Magnesium stearate | 0.5 |
| Talc | 1 |

The active ingredients, starch, and cellulose are passed through a No. 45 mesh U.S. sieve and mixed thoroughly. The solution of polyvinylpyrrolidone is mixed with the resultant powders which are then passed through a No. 14 mesh U.S. sieve. The granules so produced are dried at 50°-60° C. and passed through a No. 18 mesh U.S. sieve. The sodium carboxymethyl starch, magnesium stearate, and talc, previously passed through a No. 60 U.S. sieve, are then added to the granules which, after mixing, are compressed on a tablet machine to yield tablets.

Suspensions each containing 0.25-100 mg of active ingredient per 5 ml dose are made as follows

Formulation 4: Suspensions

| Ingredient | Quantity (mg/5 ml) |
|---|---|
| Active ingredient | 0.25-100 mg |
| Sodium carboxymethyl cellulose | 50 mg |
| Syrup | 1.25 mg |
| Benzoic acid solution | 0.10 mL |
| Flavor | q.v. |
| Color | q.v. |
| Purified Water to | 5 mL |

The active ingredient is passed through a No. 45 mesh U.S. sieve and mixed with the sodium carboxymethyl cellulose and syrup to form smooth paste. The benzoic acid solution, flavor, and color are diluted with some of the water and added, with stirring. Sufficient water is then added to produce the required volume.

An aerosol solution is prepared containing the following ingredients.

Formulation 5: Aerosol

| Ingredient | Quantity (% by weight) |
|---|---|
| Active ingredient | 0.25 |
| Ethanol | 25.75 |
| Propellant 22 (Chlorodifluoromethane) | 70.00 |

The active ingredient is mixed with ethanol and the mixture added to a portion of the propellant 22, cooled to 30° C., and transferred to a filling device. The required amount is then fed to a stainless steel container and diluted with the remaining propellant. The valve units are then fitted to the container.

Suppositories are prepared as follows:

Formulation 6: Suppositories

| Ingredient | Quantity (mg/suppository) |
|---|---|
| Active ingredient | 250 |
| Saturated fatty acid glycerides | 2,000 |

The active ingredient is passed through a No 60 mesh U.S. sieve and suspended in the saturated fatty acid glycerides previously melted using the minimal necessary heat. The mixture is then poured into a suppository mold of nominal 2 g capacity and allowed to cool.

An intravenous formulation is prepared as follows:

Formulation 7: Intravenous Solution

| Ingredient | Quantity | |
|---|---|---|
| Active ingredient dissolved in ethanol 1% | 20 | mg |
| Intralipid ™ emulsion | 1,000 | mL |

The solution of the above ingredients is intravenously administered to a patient at a rate of about 1 mL per minute. Soft gelatin capsules are prepared using the following:

Formulation 8: Soft Gelatin Capsule with Oil Formulation

| Ingredient | Quantity (mg/capsule) |
|---|---|
| Active ingredient | 10-500 |
| Olive Oil or Miglyol ™ Oil | 500-1000 |

The active ingredient above may also be a combination of agents.

### GENERAL EXPERIMENTAL PROCEDURES

The following examples are put forth so as to provide those of ordinary skill in the art with a disclosure and description of how the compounds, compositions, and methods claimed herein are made and evaluated, and are intended to be purely exemplary of the invention and are not intended to limit the scope of what the inventors regard as their invention. Unless indicated otherwise, percent is percent by weight given the component and the total weight of the composition, temperature is in ° C. or is at ambient temperature, and pressure is at or near atmospheric. Commercial reagents were utilized without further purification. Room or ambient temperature refers to 20-25° C. All non-aqueous reactions were run under a nitrogen atmosphere for convenience and to maximize yields. Concentration in vacuo means that a rotary evaporator was used. The names for the compounds of the invention were created by the Autonom 2.0 PC-batch version from Beilstein informationssysteme GmbH (ISBN 3-89536-976-4). The chemical structures depicted may be only exemplary of the general structure or of limited isomers, and not include specific stereochemistry as recited in the chemical name.

NMR spectra were recorded on a Varian Unity 400 (Varian Co., Palo Alto, Calif.) NMR spectrometer at ambient temperature. Chemical shifts are expressed in parts per million (δ) relative to an external standard (tetramethylsilane). The peak shapes are denoted as follows: s, singlet; d, doublet, t, triplet, q, quartet, m, multiplet with the prefix br indicating a broadened signal. The coupling constant (J) data given have a maximum error of ±0.41 Hz due to the digitization of the spectra that are acquired. Mass spectra were obtained by (1) atmospheric pressure chemical ionization (APCI) in alternating positive and negative ion mode using a Fisons Platform II Spectrometer or a Micromass MZD Spectrometer (Micromass, Manchester, UK) or (2) electrospray ionization in alternating positive and negative ion mode using a Micromass MZD Spectrometer Micromass, Manchester, UK) with a Gilson LC-MS interface (Gilson Instruments. Middleton, Wis.) or (3) a QP-8000 mass spectrometer (Shimadzu Corporation, Kyoto, Japan) operating in positive or negative single ion monitoring mode, utilizing electrospray ionization or atmospheric pressure chemical ionization. Where the intensity of chlorine- or bromine-containing ions are described, the expected intensity ratio was observed (approximately 3:1 for ³⁵Cl/³⁷Cl-containing ions and 1:1 for ⁷⁹Br/⁸¹Br-containing ions) and the position of only the lower mass ion is given.

Column chromatography was performed with either Baker Silica Gel (40 μm) (J. T. Baker. Phillipsburg, N.J.) or Silica Gel 60 (40-63 μm)(EM Sciences, Gibbstown, N.J.). Flash chromatography was performed using a Flash 12 or Flash 40 column (Biotage, Dyar Corp., Charlottesville, Va.). Radial chromatography was performed using a chromatotron Model 7924T (Harrison Research, Palo Alto, Calif.). Preparative HPLC purification was performed on a Shimadzu 10A preparative HPLC system (Shimadzu Corporation, Kyoto, Japan) using a model SIL-10A autosampler and model 8A HPLC pumps. Preparative HPLC-MS was performed on an identical system, modified with a QP-8000 mass spectrometer operating in positive or negative single ion monitoring mode, utilizing elecrospray ionization or atmospheric pressure chemical ionization. Elution was carried out using water/acetonitrile gradients containing either 0.1% formic acid or ammonium hydroxide as a modifier. In acidic mode, typical columns used include Waters Symmetry C8, 5 μm, 19×50 mm or 30×50 mm, Waters XTerra C18, 5 μm, 50×50 (Waters Corp, Milford, Mass.) or Phenomenex Synergi Max-RP 4 μm, 50×50 mm (Phenomenex Inc. Torrance, Calif.). In basic mode, the Phenomenex Synergi Max-RP 4 μm, 21.2×50 mm or 30×50 mm columns (Phenomenex Inc., Torrance, Calif.) were used.

Optical rotations were determined using a Jasco P-1020 Polanmeter Jasco Inc., Easton, Md.)

Dimpthylformamide (“DMF”), tetrahydrofuran (“THF”), toluene and dichloromethane (“DCM”) were the anhydrous grade supplied by Aldrich Chemical Company (Milwaukee, Wis.). Unless otherwise specified, reagents were used as obtained from commercial sources. The terms “concentrated” and “evaporated” refer to removal of solvent at 1-200 mm of mercury pressure on a rotary evaporator with a bath temperature of less than 45° C. The abbreviation “min” stand for “minutes” and “h” or “hr” stand for “hours.” The abbreviation “gm” or “g” stand for grams. The abbreviation “μl” or “μL” stand for microliters.

### Preparation 1

### (2R,4S)-(4-Benzyloxycarbonylamino-2-ethyl-6-trifluoromethyl-3,4-dihydro-2H-quinoline-1-cabonyl)-cyclohexyl]-acetic acid ethyl ester

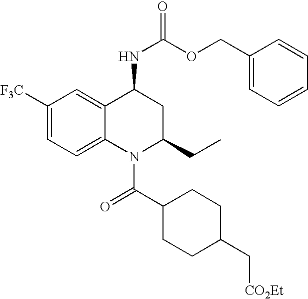

(2R,4S)-(2-Ethyl-6-trifluoromethyl-1,2,3,4-tetrahydroquinolin-4-yl-carbamic acid benzyl ester (4.0 g, 10.6 mmol) (see U.S. Pat. No. 6,706,881 for preparation information) &as added to a dry round bottomed flask equipped with a magnetic stir bar. Methylene chloride (25 mL) was added to the flask followed by pyridine (2.5 g, 31.8 mmol). To this solution, (4-chlorocarbonyl-cyclohexyl) acetic acid ethyl ester (2.5 g 21.2 mmol) in 5 mL of methylene chloride was added dropwise at 20° C. to 30° C. After 24 hours, the reaction mixture was quenched with 1.0 N HCl and the organic layer was collected. The organic layer was washed twice with NaHCO₃ solution and once with a brine solution. The organic layers were collected, dried over sodium sulfate, filtered and concentrated to dryness to provide the title compound (5.70 g) which was carried forward without further purification. MS: 575 [M+H]⁺

¹H-NMR (CDCl₃) δ: 7.65 (m, 2H), 7.40 (d, 5H), 7.25 (br s, 1H), 5.25 (s, 2H), 4.99 (d, 1H), 5.8 (br s, 1H) 5.65 (br s, 1H), (q, 2H); 3.90 (m, 1H), 2.60 (m, 2H), 2.10-2.21 (d, 2H), 1.2 (t, 3H), 0.95 (t, 3H).

### Preparation 2

### (2R,4S)-[4-(4-Amino-2-ethyl-6-trifluoromethyl-3,4-dihydro-2H-quinoline-1-carbonyl)-cyclohexyl]-acetic acid ethyl ester

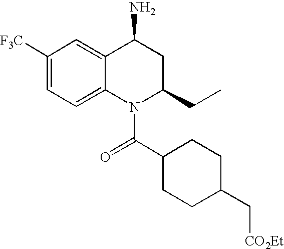

(2R,4S)-[4-(4-Benzyloxycarbonylamino-2-ethyl-6-trifluoromethyl-3,4-dihydro-2H-quinoline-1-carbonyl)-cyclohexyl]-acetic acid ethyl ester from preparation 1 (0.63 g 1.11 mmol) was added to a dry round bottomed flask equipped with a magnetic stir bar. Methanol (5 mL) was added to the flask followed by NH₄CO₂H (0.21 g, 3.33 mmol, 3.0 eq). After stirring under nitrogen, Pd/C (0.03, 0.03 mmol, 0.03 eq) was added and the reaction was heated at 45° C. for 5 hours. The reaction mixture was quenched with water and extracted 3 times with ethyl acetate. The organic layers were collected, dried over sodium sulfate, filtered and concentrated to dryness to provide the title compound (0.46 g) which was carried forward without further purification. MS: 441 [M+H]⁺

¹H-NMR (CDCl₃) δ: 7.95 (s, 1H), 7.65 (d, 1H), 7.25 (brs, 1H, 4.86 (q, 2H), 3.90 (m, 1H), 2.60 (m, 2H), 2.10-2.21 (d, 2H), 1.2 (m, 3H), 0.95 (m, 3H).

### Preparation 3

### (2R,4S)-[4-(4-(3,5-Bistrifluromethyl-benzylamino)-2-ethyl-6-trifluoromethyl-3,4-dihydro-2H-quinoline-1-carbonyl)-cyclohexyl]-acetic acid ethyl ester

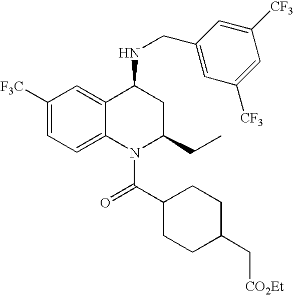

To a solution of (2R,4S)-[4-(4-Amino-2-ethyl-6-trifluoromethyl-3,4-dihydro-2H-quinoline-1-carbonyl)-cyclohexyl]-acetic acid ethyl ester from preparation 2 (1.0 g, 2.3 mmol) in methylene chloride (20 mL) was added 3,5-bis(trifluoromethyl)benzaldehyde. The mixture was stirred at 30° C. for 2 hours. At this time, solid sodium triacetoxyborohydride (2.4 g, 11.4 mmol) was added and the reaction was stirred for 12 hours. The reaction was quenched with 2N KOH and diluted with water. The organic layer was dried over anhydrous magnesium sulfate, filtered, evaporated to dryness to provide a crude oil which was purified by chromatography using silica to afford the title compound. MS: 667 [M+H]⁺ found

¹H-NMR (COCl₃) δ: 7.89 (s, 2H), 7.83 (s, 1H), 7.80 (s, 1H), 7.56 (d, 1H), 7.20 (bd, 1H), 4.74 (q, 2H), 4.1 (m, 4H), 3.46 (m, 1H), 2.75 (m, 1H), 2.54 (m, 1H), 2.11 (d 2H), 1.9-1.3 (m, 12H), 1.22 (t 3H), 0.83 (t, 3H),

### Example 1

### (2R,4S)-[4-(4-3,5-Bis-trifluoromethyl-benzylcyanamide)-2-ethyl-6-trifluoromethyl-3,4-dihydro-2H-quinoline-1-carbonyl-cyclohexyl]-acetic acid ethyl ester

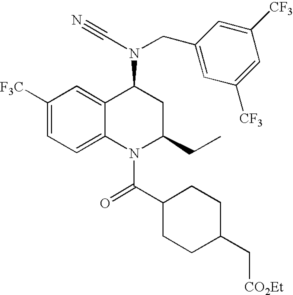

To a solution of (2R,4S)-[4-(4-(3,5-Bis-trifluoromethyl-benzylamino)-2-ethyl-6-trifluoro-methyl-3,4-dihydro-2H-quinoline-1-carbonyl)-cyclohexyl]-acetic acid ethyl ester from preparation 3 (1.0 g, 21.66 mmol) in methanol (10 mL) was added NaOAc, and BrCN The mixture was stirred at 30° C. for 12 hours. At this time, the solvent was removed, and the residue was taken up in ethyl acetate, washed with 500 mL water, dried over magnesium sulfate, filtered and concentrated to dryness to provide the title compound that was used without further purification. MS: 692 [M+H]⁺ found

¹H-NMR (CDCl₃) δ: 7.93 (s, 1H), 7.84 (s, 2H) 7.60 (s, 1H), 7.59 (d, 1H), 7.28 (br d, 1H), 4.70 (br s, 1H), 4.65 (d, 1H), 4.53 (d, 1H), 4.10 (q, 3H), 3.69 (m, 1H), 2.69 (m, 1H), 2.49 (m, 1H), 2.12 (d, 2H), 1.9-1.3 (m, 12H, 1.23 (t, 3H), 0.84 (t, 3H).

### Example 2

### (2R,4S)-4-{4-[(3,5-Bis-trifluoromethyl-benzyl)-(2H-tetrazol-5-yl)-amino]-2-ethyl-6-trifluoromethyl-3,4-dihydro-2H-quinoline-1-carbonyl}-cyclohexyl)-acetic acid ethyl ester

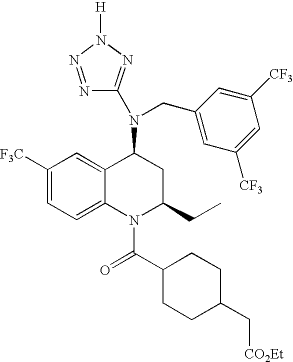

To a solution of (2R,4S)-[4-(4-(3,5 Bis-trifluoromethyl-benzylcyanamide)-2-ethyl-6-trifluoromethyl-3,4-dihydro-2H-quinoline-1-carbonyl)-cyclohexyl]-acetic acid ethyl ester from example 1 (0,400 g, 0.58 mmol) in toluene (15 mL) was added to a 65 ml flask containing a magnetic stirbar and reflux condenser. To this solution, sodium azide and triethylamine hydrochloride were added. The mixture was stirred at 100° C. for 24 hours. At this time, the reaction was cooled to 30° C. The solvent was removed, and the residue was taken up in ethyl acetate, washed with 500 mL water, dried over magnesium sulfate, filtered and concentrated to dry ness to provide the title compound that was used without further purification. MS. 735 [M+H]⁺ found

¹H-NMR (CDCl₃) δ: 7.80 (bs, 3H), 7.59 (br d, 1H), 7.29 (br d, 1H), 7.21 (s, 1H), 5.25 (br s, 1H), 4.8 (br s, 1H), 4.10 (q, 2H), 2.60 (br s, 2H), 2.10 (m, 1H), 1.30 (t, 3H), 0.96 (t, 3H).

### Examples 3 and 4

### Trans-(2R,4S)- and Cis-(2R,4S)-(4-{4-[(3,5-Bis-trifluoromethyl-benzyl)-(2-methyl-2H-tetrazol-5-yl)-amino]-2-ethyl-6-trifluoromethyl-3,4-dihydro-2H-quinoline-1-carbonyl}-cyclohexyl)-acetic acid ethyl ester

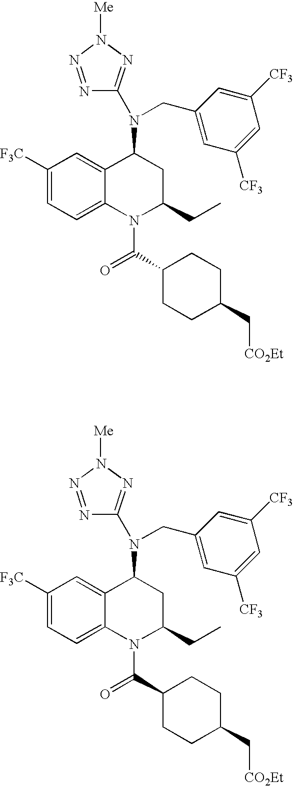

To a solution of (2R,4S)-4-{4-[(3,5-Bis-trifluoromethy-benzyl)-(2H-tetrazol-5-yl)-amino]-2-ethyl-6-trifluoromethyl-3,4-dihydro-2H-quinoline-1-carbonyl}-cyclohexyl)-acetic acid ethyl ester from example 2 (0.25 mg) in DMSO (20 mL) was added K₂CO₃ (1.0 g) followed by methyl iodide (2.0 ml). The mixture was stirred at 30° C. for 24 hours. At this time, the reaction was quenched with 50 ml of water and extracted with ethyl acetate. The organic layer was collected, dried over magnesium sulfate, filtered and concentrated to dryness to provide a crude mixture which was purified by chromatography using silica to afford the title compound as a major (trans cyclohexane) and minor isomer (cis cyclohexane).

Trans cyclohexane isomer: (2R,4S)-(4-{4-[(3,5-Bis-trifluoromethyl-benzyl)-(2-methyl-2H-tetrazol-5-yl)amino]-2-ethyl-6-trifluoromethyl-3,4-dihydro-2H-quinoline-1-carbonyl}-cyclohexyl)-acetic acid ethyl ester MS: 749 [M+H]⁺ found. ¹H-NMR (CDCl₃)δ: 7.78 (bs, 3H), 7.56 (br d, 1H), 7.27 (br d, 1H), 7.17 (s, 1H), 5.12 (br d, 1H), 4.75 (br s, 1H), 4.63 (br s, 1H), 4.17 (s, 3H), 4.10 (q, 2H), 2.54 (br s, 1H), 2.44 (brs, 1H), 2.13 (d, 2H) 1.23 (t, 3H), 0.78 (t, 3H).

Cis cyclohexane isomer: (2R,4S)-(4-{4-[(3,5-Bis-trifluoromethyl-benzyl)-(2-methyl-2H-tetrazol-5-yl)-amino]-2-ethyl-6-trifluoromethyl-3,4-dihydro-2H-quinoline-1-carbonyl-cyclohexyl)-acetic acid ethyl ester MS: 749 [M+H]⁺ found. ¹H-NMR (CDCl₃)δ: 7.78 (bs, 3H), 7.56 (br d, 1H), 7.27 (br d, 1H), 7.17 (s, 1H), 5.13 (br d, 1H), 4.74 (br s, 1H), 4.63 (br s, 1H), 4.17 (s, 3H), 4.10 (q, 2H), 2.75 (br s, 1H), 2.44 (br s, 1H), 2.35 (br s, 1H), 2.12 (br s, 1H) 1.24 (t, 3H), 0.78 (t, 5H).

In an alternative procedure, to a solution of trans-(2R,4S)-[4-{4-(3,5-bis-trifluoromethyl-benzylamino-2-ethyl-6-trifluoromethyl-3,4-dihydro-2H-quinoline-1-carbonyl}-cyclohexyl]-acetic acid ethyl ester (500 mg) and sodium acetate (185 mg) in 5 ml of methanol was added 500 μL of 3 M cyanogen bromide in dichloromethane. The reaction mixture was allowed to stir at ambient temperature until starting material was consumed. The reaction mixture was diluted with 10 ml of 2-methyltetrahydrofuran and 10 ml of water. The layers were separated and the upper product rich organic phase was dried over sodium sulfate, filtered, and used in the next step without further purification.

To the reaction solution from the previous step was added 500 μL of triethylamine and 200 μL of azidotrimethylsilane. The reaction mixture was stirred at ambient temperature until the starting material was consumed. Dimethylformamide (1.0 mL) and 90.0 μL of methyl iodide were added to the reaction mixture, followed by stirring at ambient temperature until the starting material vas consumed. The crude reaction mixture was then diluted with 10 ml of water and the layers were separated. The upper product rich organic layer was dried over sodium sulfate, filtered, and the solvent was removed in vacuo to afford 480 mg of a 95:5 mixture of trans-(2R,4S)-(4-{4-[(3,5-Bis-trifluoromethyl-benzyl)-(2-methyl-2H-tetrazol-5-yl) -amino]-2-ethyl-6-trifluoromethyl-3,4-dihydro-2H-quinoline-1-carbonyl}-cyclohexyl)acetic acid ethyl ester; trans-(2R,4S)-(4-{4-[(3,5-Bis-trifluoromethyl-benzyl)-(1 methyl-2H-tetrazol-5-yl)amino]-2-ethyl-6-trifluoromethyl-3,4-dihydro-2H-quinoline-1-carbonyl}-cyclohexyl)-acetic acid ethyl ester (90%).

### Examples 5 and 6

### Trans-(2R,4S)- and Cis-(2R,4S)-(4-{4-[(3,5-Bis-trifluoromethyl-benzyl)-(2-methyl-2H-tetrazol-5-yl)-amino]-2-ethyl-6-trifluoromethyl-3,4-dihydro-2H-quinoline-1-carbonyl}-cyclohexyl)-acetic acid

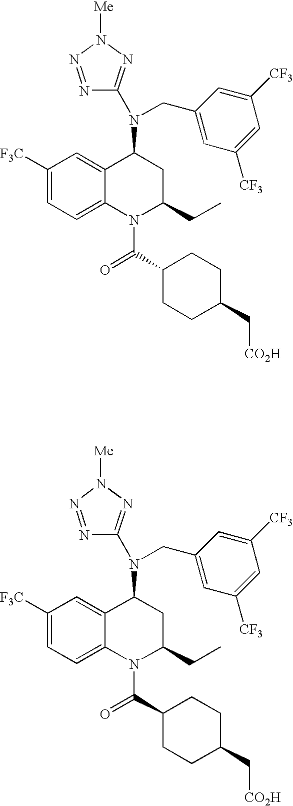

To a solution of (2R,4S)-4-{4-[(3,5-Bis-trifluoromethyl-benzyl)-(2-methyl-2H-tetrazol-5-yl)-amino]-2-ethyl-6-trifluoromethyl-3,4-dihydro-2H-quinoline-1-carbonyl}-cyclohexyl)-acetic acid ethyl ester from example 3 (0.200 mg) in ethanol (5 mL) was added 4.0N potassium hydroxide (5 ml) and the reaction was stirred at 60° C. for 2 hours. At this time, the solvent was removed and the residue was taken up in water and extracted with ether. The aqueous layer was acidified with citric acid (1M) and extracted into ethyl acetate. The organic extracts were dried over magnesium sulfate, filtered and concentrated to dryness to provide a the title compound as a white solid that was used without further purification.

Trans cyclohexane isomer: (2R,4S)-(4-{4-[(3,5-Bis-trifluoromethyl-benzyl)-(2-methyl-2H-tetrazol-5-yl)-amino]-2-ethyl-6-trifluoromethyl-3,4-dihydro-2H-quinoline-1-carbonyl}-cyclohexyl)-acetic acid MS: 722 [M+H]⁺ found. ¹H-NMR (CDCl₃)δ: 7.78 (bs, 3H), 7.56 (br d, 1H), 7.27 (br d, 1H), 7.17 (s, 1H), 5.12 (br d, 1H), 4.75 (br s, 1H), 4.63 (br s, 1H), 4.17 (br s, 3H), 2.55 (br s, 1H), 2.44 (br s, 1H), 2.19 (d 2H), 0.78 (t, 3H).

Cis cyclohexane isomer: (2R,4S)-(4-{4-[(3,5-Bis-trifluoromethyl-benzyl)-(2-methyl-2H-tetrazol-5-yl)-amino]-2-ethyl-6-trifluoromethyl-3,4-dihydro-2H-quinoline-1-carbonyl}-cyclohexyl)-acetic acid

MS: 722 [M+H]⁺ found. ¹H-NMR (CDCl₃)δ: 7.78 (bs, 3H), 7.56 (br d, 1H), 7.27 (br d, 1H), 7.17 (s, 1H), 5.12 (br d, 1H), 4.75 (br s, 1H), 4.63 (br s, 1H), 4.17 (s, 3H), 2.76 (br s, 1H), 2.44 (br s, 1H), 2.41 (d, 2H), 0.78 (t, 3H).

Examples 7-10 were prepared in an analogous fashion to the above Examples using the appropriate starting materials.

### Example 7

### Trans-2R,4S)-4-{4-[(3,5-Bis-trifluoromethyl-benzyl)-methoxycarbonyl-amino]-2-ethyl-6-trifluoromethyl-3,4-dihydro-2H-quinoline-1-carbonyl}-cylcohexyl)-acetic acid

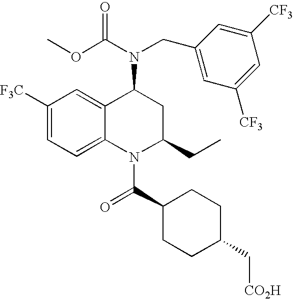

MS: 697 [M+H]⁺ found. ¹H-NMR (CDCl₃) δ: 7.79 (bs, 3H), 3.78 (s, 3H) 0.63 (t, 3H).

### Example 8

### (2R,4S)-4-[(3,5-Bis-trifluoromethyl-benzyl)-(2-methyl-2H-tetrazol-5-yl)-amino]-2-ethyl-6-trifluoromethyl-3,4-dihydro-2H-quinoline-1-carboxylic add isopropyl ester

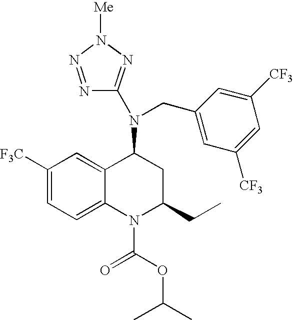

MS: 639 [M+H]⁺ found. ¹H-NMR (CDCl₃)δ: 7.79 (bs, 3H), 7.61 (d, 1H), 7.50 (d, 1H), 7.07 (s, 1H), 5.12 (br s, 1H), 5.03 (hept, 1H), 4.50 (br m, 2H), 4.63 (br s, 1H), 4.17 (s, 3H), 2.76 (br s, 1H), 2.44 (br s, 1H), 2.41 (d, 2H), 0.78 (t, 3H).

### Example 9

### Trans-(2R,4S)-4-{4-[(3,5-Bis-trifluoromethyl-benzyl)-(2-methyl-2H-tetrazol-5-yl)-amino]-2-ethyl-6-trifluoromethyl-3,4-dihydro-2H-quinoline-1-carbonyl}-cyclohexanecarboxylic acid methyl ester

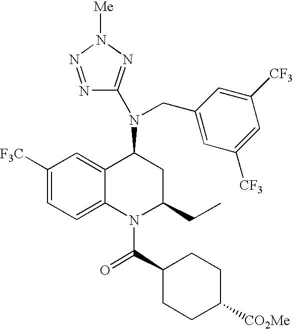

MS: 721 [M+H]⁺ found. ¹H-NMR (CDCl₃)δ: 7.79 (bs, 3H), 7.57 (d, 1H), 7.27 (d, 1H), 7.18 (s, 1H), 5.12 (br d, 1H), 4.75 (m, 1H), 4.6 (m, 1H), 4.17 (s, 3H), 3.54 (s, 3H), 2.59 (m, 1H), 2.43 (m, 1H), 2.32 (m, 1H), 0.78 (t, 3H).

### Example 10

### Trans-(3R,4S)-4-{4-[(3,5-Bis-trifluoromethyl-benzyl)-(2-methyl-2H-tetrazol-5-yl)-amino]-2-ethyl-6-trifluoromethyl-3,4-dihydro-2H-quinoline-1-carbonyl}-cyclohexanecarboxylic acid

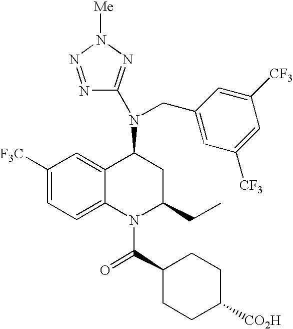

MS: 707 [M+H]⁺ found, ¹H-NMR (CDCl₃) δ: 7.75 (bs, 3H), 7.54 (br d, 1H), 7.24 (br d, 1H), 7.15 (s 1H), 5.10 (br d, 1H), 4.73 (m, 1H), 4.6 (m, 1H), 4.14 (s, 3H) 2.56 (m, 1H), 2.41 (m, 1H), 2.31 (m, 1H) 0.75 (t, 3H).

### Example 11

### (2R,4R)-4-[(3,5-Bis-trifluoromethyl-benzyl)-(2-methyl-2H-tetrazol-5-yl)-amino]-carboxylic acid ethyl ester

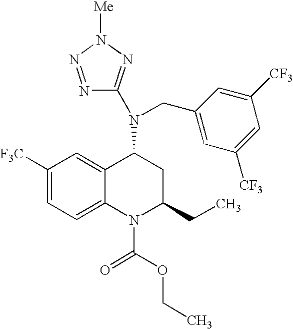

(2R,4S)-4-Chloro-2-ethyl-6-trifluoromethyl-3,4-dihydro-2H-quinoline-1-carboxylic acid ethyl ester (200 mg) and (3,5-Bis-trifluoromethyl-benzyl)-(2-methyl-2H-tetrazol-5-yl)-amine (220 mg) were combined in 5 mL of DMF and cooled in an ice water bath as sodium hexamethyldisilazide (0.78 mL of a 1.0M solution in THF) was added slowly. After stirring 30 min, the cooling bath was removed and the mixture allowed to warm to room temperature. After 30 min, the reaction was quenched with a saturated aqueous ammonium chloride solution and extracted with ethyl acetate. The combined organic layers were dried over magnesium sulfate, filtered, and concentrated. The residue was purified by chromatography on silica eluting with an ethyl acetate-hexanes mixture to afford the title compound.

MS: 625 [M+H]⁺ found. ¹H-NMR (CDCl₃)δ: 7.80 (d, 1H), 7.68 (s, 1H), 7.54 (s, 2H) 7.39 (d, 1H), 7.27 (s, 1H), 5.70 (dd, 1H), 4.68 (m, 1H), 4.54 (d, 1H), 4.3 (m, 3H), 4.20 (s, 3H), 2.29 (m, 1H), 2.12 (m, 1H), 1.55 (m, 2H), 1.35 (t, 3H), 0.92 (t, 3H).

### Example 12

### Trans-(2R,4S)-2-(4-{4-[(3,5-Bis-trifluoromethyl-benzyl)-(2-methyl-2H-tetrazol-5-yl)-amino]-2-ethyl-6-trifluoromethyl-3,4-dihydro-2H-quinoline-1-carbonyl}-cyclohexyl)-acetamide

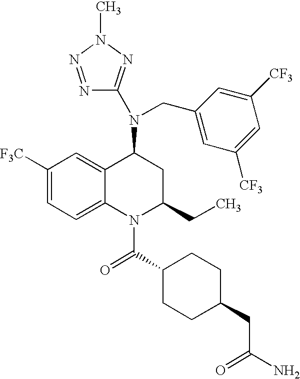

Trans-(2R,4S)-(4-{4-[(3,5-Bis-trifluoromethyl-benzyl)-(2-methyl-2H-tetrazol-5-yl)-amino)]-2-ethyl-6-trifluoromethyl-3,4-dihydro-2H-quinoline-1-carbonyl}-cyclohexyl)-acetic acid (1.0 g) was dissolved in 0.5 mL of thionylchloride, stirred at ambient temperature for 3 hours, the volatiles removed under reduced pressure, and the residue dissolved in 20 mL of THF. The resulting solution was cooled in a dry ice/acetone bath as gaseous ammonia was condensed into the mixture until it was saturated. After warming to room temperature, the resulting reaction mixture was treated with 5 mL of 1N HCl and extracted with ethyl acetate. The combined organic layers were dried over MgSO4, filtered and concentrated under vacuum to afford the crude product, which was purified by silica gel chromatography, eluting with ethyl acetate, to afford the title compound. MS: 721 [M+H]⁺ found. ¹H-NMR (CDCl₃) δ: 7.78 (s, 1H), 7.76 (s, 2H), 7.55 (d, 1H), 7.25 (d, 1H), 7.16 (s, 1H), 5.39 (br s, 1H), 5.36 (br s, 1H), 5.10 (br d, 1H) 4.74 (m, 1H), 4.60 (m, 1H), 4.16 (s, 3H), 2.51 (m, 1H), 2.42 (m, 1H), 2.04 (d, 2H), 0.76 (t, 3H).

### Example 13

### Trans-(2R,4S)-(4-{4-[(3,5-Bis-trifluoromethyl-benzyl)-methoxycarbonyl-amino]-2-ethyl-6-trifluoromethyl-3,4-dihydro-2H-quinoline-1-carbonyl}-cyclohexyl)-acetic acid ethyl ester

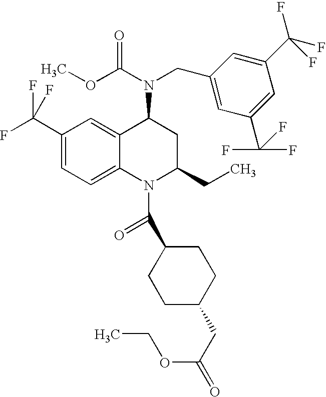

To a solution of trans-(2R,4S)-[4-(4-(3,5-bis-trifluoromethyl-benzylamino)-2-ethyl-6-trifluoro-methyl-3,4-dihydro-2H-quinoline-1-carbonyl}-cyclohexyl)-acetic acid ethyl ester from preparation 3 (0.5 g) in dichloromethane (10 mL) was added pyridine (1.0 ml) and methylchloroformate (1.0 ml). After 18 hours, the reaction mixture was treated with 1N HCl and extracted with dichloromethane. The combined organic phases were dried over magnesium sulfate, filtered and concentrated to dryness to provide the crude mixture, which was purified by chromatography on silica eluting with 5-10% ethyl acetate in hexanes to provide the title compound (400 mg). MS: 725 [M+H]⁺ found

### Example 14

### Trans-(2R,4S)-(3,5-Bis-trifluoromethyl-benzyl)-[1-(4-carbamoylmethyl-cyclohexanecarbonyl)-2-ethyl-6-trifluoromethyl-1,2,3,4-tetrahydro-quinolin-4-yl]-carbamic acid methyl ester

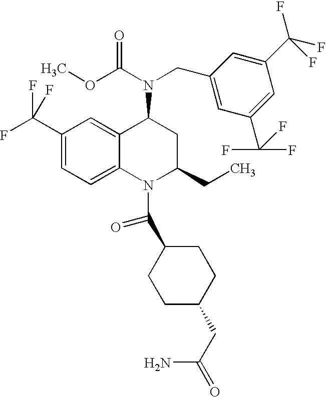

Trans-(2R,4S)-(4-{4-[(3,5-bis-trifluoromethyl-benzyl)-methoxycarbonyl-amino]-2-ethyl-6-trifluoromethyl-3,4-dihydro-2H-quinoline-1-carbonyl}-cyclohexyl)-acetic acid (100 mg) was dissolved in tetrahydrofuran (5 ml) and treated with 1.0 mL of thionylchloride. After the reaction mixture was stirred at ambient temperature for 3 hours, the volatiles removed under reduced pressure, and the residue dissolved in 15 mL of THF. The resulting solution was cooled in a dry ice/acetone bath as gaseous ammonia was condensed into the mixture until it vas saturated. After warming to room temperature for 2 hours, the resulting reaction mixture was treated with 5 mL of 1N HCl and extracted with ethyl acetate. The combined organic layers were dried over MgSO4, filtered and concentrated under vacuum to afford the crude product, which was purified by silica gel chromatography, eluting with ethyl acetate, to afford 7 mg of the title compound. MS: 696 [M+H]⁺ found. ¹H-NMR (CDCl₃) δ: 7.79 (s, 1H), 7.72 (s, H), 7.66 (s, 1H), 7.57 (s, 1H), 7.22 (br s, 2H).

### Example 15-17

### Trans-(2R,4S)-{4-[(3,5-Bis-trifluoromethyl-benzyl)-(2-methyl-2H-tetrazol-5-yl)-amino]-2-ethyl-6-trifluoromethyl-3,4-dihydro-2H-quinolin-1-yl}-4-(2-hydroxy-ethyl)-cycohexyl]-methanone

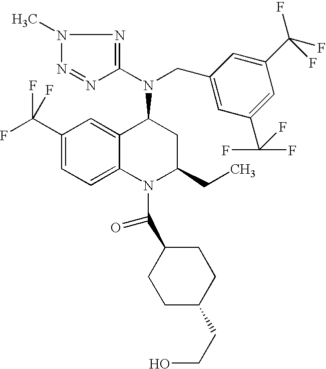

### Trans-[2R,4S)-2-(4-{4-[(3,5-Bis-trifluoromethyl-benzyl)-(2-methyl-2H-tetrazol-5-yl)-amino]-2-ethyl-6-trifluoromethyl-3,4-dihydro-2H-quinolin-1-ylmethyl}-cyclohexyl)-ethanol

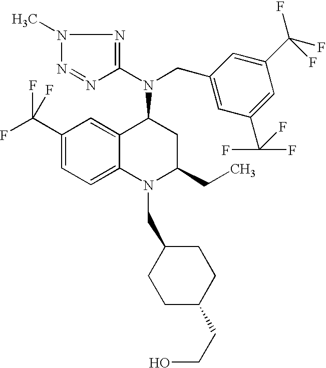

### Trans-(2R,4S)-(4-{4-[(3,5-Bis-trifluoromethyl-benzyl)-(2-methyl-2H-tetrazol-5-yl)-amino]-2-ethyl-6-trifluoromethyl-3,4-dihydro-2H-quinolin-1-ylmethyl}-cyclohexyl)-acetic acid ethyl ester

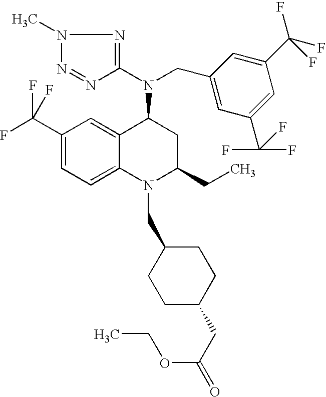

Trans-(2R,4S)-(4-{4-[(3,5-Bis-trifluoromethyl-benzyl)-(2-methyl-2H-tetrazol-5-yl)-amino]-2-ethyl-6-trifluoromethyl-3,4-dihydro-2H-quinoline-1-carbonyl}-cyclohexyl)-acetic acid ethyl ester (Example 3) (720 mg) in 10 mL of THF was treated at room temperature with borane dimethylsulfide (1.5 mL of a 2M soln). After 3 days, the reaction mixture was concentrated under vacuum and the resulting residue quenched with 5 mL of ethyl alcohol. The resulting mixture was diluted with water and extracted with ethyl acetate. The combined organic layers were dried over magnesium sulfate, filtered, and concentrated under reduced pressure to afford the mixture of products. The product mixture was separated by chromatography on silica gel eluting with 15% ethyl acetate in hexanes to afford the title compounds.

Trans-(2R,4S)-{4-[(3,5-Bis-trifluoromethyl-benzyl)-(2-methyl-2H-tetrazol-5-yl)-amino]-2-ethyl-6-trifluoromethyl-3,4-dihydro-2H-quinolin-1-yl}-[4-(2-hydroxy-ethyl)-cyclohexyl]-methanone. MS: 707 [M+H]⁺ found. ¹H-NMR (CDCl₃) δ: 7.79 (s, 1H), 7.78 (s, 2H), 7.57 (d, 1H), 7.27 (d, 1H), 7.18 (s, 1H), 5.12 (br d, 1H) 4.75 (m, 1H), 4.60 (m, 1H), 4.17 (s, 3H), 3.66 (t, 2R), 2.55 (m, 1H), 2.44 (m, 1H), 0.78 (t, 3H).

Trans-(2R,4S)-2-(4-{4-[(3,5-Bis-trifluoromethyl-benzyl)-(2-methyl-2H-tetrazol-5-yl)-amino]-2-ethyl-6-trifluoromethyl-3,4-dihydro-2H-quinolin-1-ylmethyl}-cyclohexyl)-ethanol. MS: 693 [M+H]⁺ found. ¹H-NMR (CDCl₃) δ: 7.75 (s, 1H), 7.70 (s, 2H), 7.29 (d, 1H), 6.96 (s, 1H), 6.67 (d, 1H), 4.76 (d, 2H), 4.45 (m, 1H), 4.20 (s, 3H), 3.68 (t, 2H), 3.55 (m, 1H), 3.40 (dd, 1H), 2.95 (dd, 1H), 0.75 (t, 3H.

Trans-2R,4S)-(4-{4-[(3,5-Bis-trifluoromethyl-benzyl)-(2-methyl-2H-tetrazol-5-yl)-amino]-2-ethyl-6-trifluoromethyl-3,4-dihydro-2H-quinolin-1-ylmethyl}-cyclohexyl)-acetic acid ethyl ester. MS: 735 [M+H]⁺ found. ¹H-NMR (CDCl₃) δ: 7.74 (s, 1H), 7.70 (s, 2H), 7.29 (d, 1H), 6.96 (s, 1H), 6.67 (d, 1H), 4.76 (d, 2H), 4.45 (m, 1H), 4.19 (s, 3H), 4.11 (q, 2H), 3.55 (m, 1H), 3.41 (dd, 1H), 2.96 (dd, 1H), 2.16 (d, 2H), 1.24 (t, 3H), 0.75 (t, 3H).

### Example 18

### Trans-(2R,4S)-(4-{4-[(3,5-Bis-trifluoromethyl-benzyl)-(2-methyl-2H-tetrazol-5-yl)-amino]-2-ethyl-6-trifluoromethyl-3,4-dihydro-2H-quinolin-1-ylmethyl}-cyclohexyl)-acetic acid

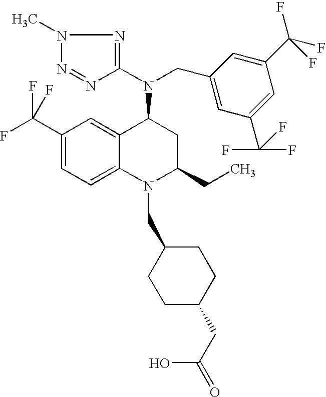

Trans-(2R,4S)-(4-{4-[(3,5-Bis-trifluoromethyl-benzyl)-(2-methyl-2H-tetrazol-5-yl)-amino]-2-ethyl-6-trifluoromethyl-3,4-dihydro-2H-quinolin-1-ylmethyl}-cyclohexyl)-acetic acid ethyl ester (150 mg) was dissolved in 5 mL of ethyl alcohol and reacted with 2 equivalents of sodium hydroxide as a 4N aqueous solution. After stirring at 60° C. for 2 hours, the reaction mixture was concentrated under reduced pressure, diluted with 10 mL of water, made acidic with a 1M citric acid solution, extracted with ethyl acetate, the combined organic layers dried over magnesium sulfate, filtered and condensed under reduced pressure to afford the title compound. MS: 707 [M+H]⁺ found. ¹H-NMR (CDCl₃) δ: 7.75 (s, 1H), 7.70 (s, 2H), 7.30 (d, 1H), 6.96 (s, 1H), 6.67 (d, 1H), 4.76 (d, 2H), 4.45 (m, 1H), 4.19 (s, 3H), 4.11 (q, 2H), 3.55 (m, 1H), 3.42 (dd, 1H), 2.96 (dd, 1H), 2.23, (d, 2H), 0.75 (t, 3H).

### Example 19

### Trans-(2R,4S)-2-(4-{4-[(3,5-Bis-trifluoromethyl-benzyl)-(2-methyl-2H-tetrazol-5-yl)-amino]-2-ethyl-6-trifluoromethyl-3,4-dihydro-2H-quinolin-1 ylmethyl}-cyclohexyl)-acetamide

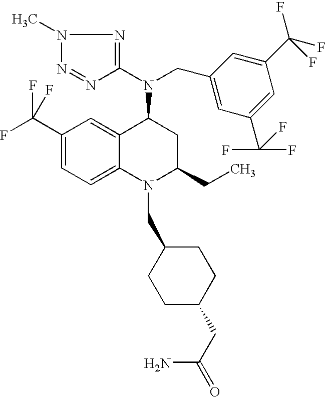

Trans-(2R,4S)-(4-{4-[(3,5-Bis-trifluoromethyl-benzyl)-(2-methyl-2H-tetrazol-5-yl)-amino]-2-ethyl-6-trifluoromethyl-3,4-dihydro-2H-quinolin-1-ylmethyl}-cyclohexyl)-acetic acid is reacted to provide the title compound using standard methods for converting a carboxylic acid to a primary amide.

### Example 20

### Trans-(2R,4S)-4-{4-[(3,5-Bis-trifluoromethyl-benzyl)-(2-methyl-2H-tetrazol-5-yl)-amino]-2-ethyl-6-trifluoromethyl-3,4-dihydro-2H-quinoline-1-carbonyl}-cyclohexanecarboxylic acid amide

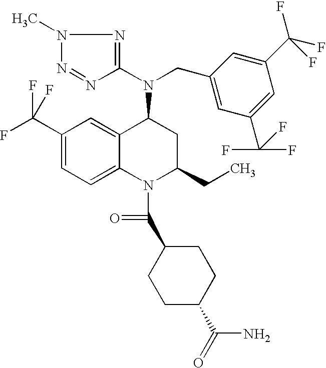

Trans-(2R,4S)-4-{4-[(3,5-Bis-trifluoromethyl-benzyl)-(2-methyl-2H-tetrazol-5-yl)-amino]-2-ethyl-6-trifluoromethyl-3,4-dihydro-2H-quinoline-1 carbonyl}-cyclohexanecarboxylic acid was reacted as for Example 19 to provide the title compound. MS: 706 [M+H]⁺ found. ¹H-NMR (CDCl₃) δ: 7.79 (s, 1H), 7.77 (s, 2H), 7.57 (d, 1H) 7.27 (br d, 1H), 7.18 (s, 1H), 5.47 (br s, 1H), 5.35 (br s, 1H), 5.12 (br d, 1H), 4.75 (m, 1H), 4.65 (m, 1H), 4.17 (s, 3H), 2.64 (m, 1H), 2.43 (m, 1H) 2.20 (m, 1H), 0.78 (t, 3H).

### Example 21

### Trans-(2R,4S)-{4-[(3,5-Bis-trifluoromethyl-benzyl)-(2-methyl-2H-tetrazol-5-yl -amino]-2-ethyl-6-trifluoromethyl-3,4-dihydro-2H-quinolin-1-yl}-[4-(1-hydroxy-1-methyl-ethyl)-cyclohexyl-methanone

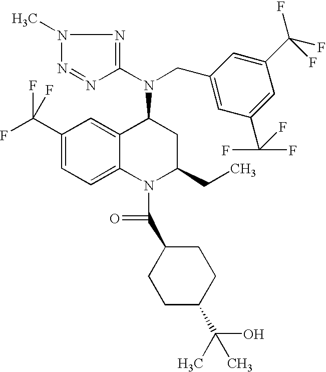

Trans-(2R,4S)-4-{4-[(3,5-Bis-trifluoromethyl-benzyl)-(2-methyl-2H-tetrazol-5-yl)-amino]-2-ethyl-6-trifluoromethyl-3,4-dihydro-2H-quinoline-1-carbonyl}-cyclohexanecarboxylic acid methyl ester Example 9 (500 mg) in 5 ml of anhydrous tetrahydrofuran was treated with methylmagnesium bromide (1.0 ml of a 1.4M solution) at room temperature. After 18 hours, the reaction mixture was treated with a saturated aqueous ammonium hydrochloride solution and extracted with ethyl acetate. The combined organic layers were dried over sodium sulfate, filtered and concentrated under reduced pressure. The crude product was purified by chromatography on silica gel eluting with 20 to 30% ethyl acetate in hexanes to afford 450 mg of the title compound. MS: 721 [M+H]⁺ found. ¹H-NMR (CDCl₃) δ: 7.79 (s, 1H), 7.78 (s, 2H), 7.58 (d, 1H), 7.27 (br d, 1H), 7.18 (s, 1H), 5.13 (br d, 1H), 4.76 (m, 1H), 4.65 (m, 1H), 4.17 (s, 3H), 2.54 (m, 1H), 2.44 (m, 1H), 1.13 (2, 6H) 0.78 (t, 3H).

Examples 22 and 23 were prepared from a procedure analogous to Example 21 using the appropriate starting materials.

### Example 22

### Trans-(2R,4S)-{4-[(3,5-Bis-trifluoromethyl-benzyl)-(2-methyl-2H-tetrazol-5-yl)-amino]-2-ethyl-6-trifluoromethyl-3,4-dihydro-2H-quinolin-1-yl}-[4-(2-hydroxy-2-methyl-propyl)-cyclohexyl]-methanone

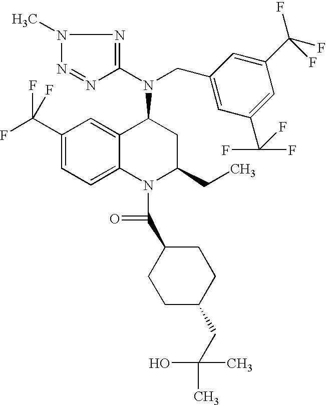

MS: 735 [M+H]⁺ found. ¹H-NMR (CDCl₃) δ: 7.78 (s, 1H), 7.76 (s, 2H), 7.55 (d, 1H), 7.25 (br d, 1H), 7.16 (s, 1H), 5.10 (br d, 1H), 4.76 (m, 1H), 4.65 (m, 1H), 4.16 (s, 3H), 2.54 (m, 1H), 2.44 (m, 1H), 1.20 (2, 6H) 0.76 (t, 3H).

### Example 23

### Trans-2R,4S)-(4-{4-[(3,5-Bis-trifluoromethyl-benzyl)-(2-methyl-2H-tetrazol-5-yl)-amino]-2-ethyl-6-trifluoromethyl-3,4-dihydro-2H-quinolin-1-ylmethyl}-cyclohexyl)-methanol

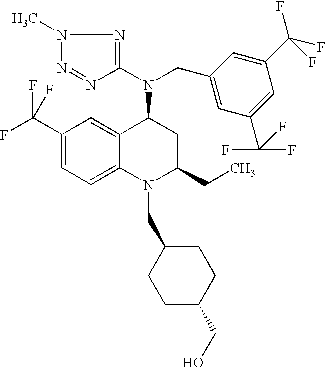

MS: 679 [M+H]⁺ found. ¹H-NMR (CDCl₃) δ: 7.75 (s, 1H), 7.70 (s, 2H), 7.30 (d, 1H), 6.98 (s 1H), 6.71 (br d, 1H), 4.76 (d, 1H), 4.45 (m, 1H), 4.19 (s, 3H), 3.66 (m, 1H), 3.44 (m, 3H), 2.98 (dd, 1H), 0.76 (t, 3H).

### Preparation 4

### Synthesis of trans-4-(Carbethoxymethyl)cyclohexanecarboxylic acid (Intermediate F) According to Scheme 5

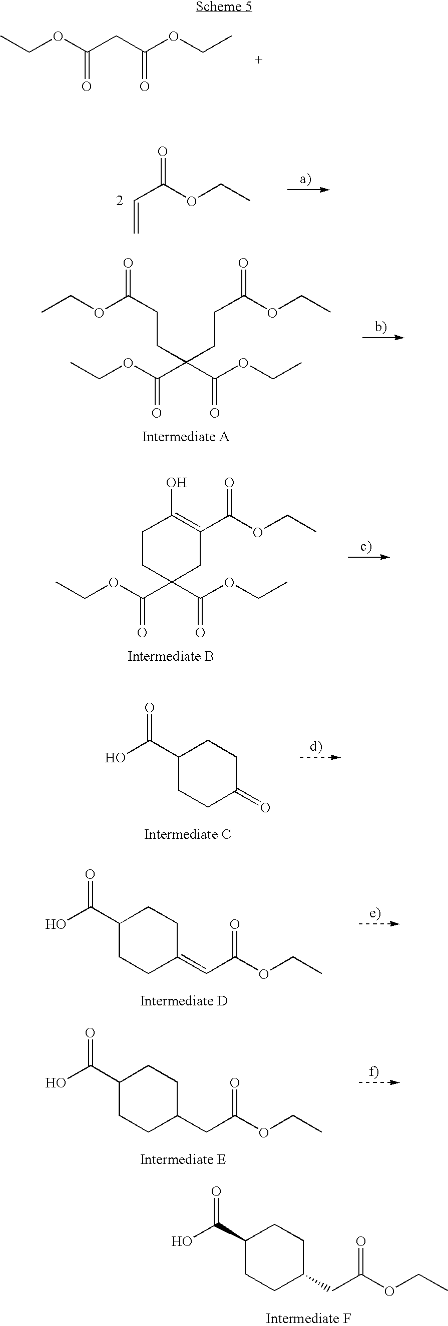

### Steps a and b) Synthesis of 4-Hydroxy-cyclohex-3-ene-1,1,3-tricarboxylic acid, triethyl ester (Intermediate B)

Sodium ethoxide (303 g, 4.45 mol, 2.25 eq) was dissolved to anhydrous ethanol (3200 ml) under nitrogen. While cooled in an ice bath, diethyl malonate (300 ml, 317 g, 1.98 mol, 1 eq) was added, followed by ethyl acrylate (428 ml, 396 g, 3.95 mol, 2 eq) at a rate in which the reaction temperature remained between 22-34° C. After the addition, the ice bath was removed and the reaction mixture was stirred overnight. The next morning, the reaction mixture was warmed up and after 30 minutes reflux, the heating mantel was removed and allowed to cool down to 35° C. The reaction mixture was cooled to 5° C. in an ice bath and 350 ml concentrated hydrochloric acid solution was added dropwise. The formed solids were removed by filtration and after the filtrate was concentrated under reduced pressure, 4-hydroxy-cyclohex-3-ene-1,1,3-tricarboxylic acid, triethyl ester (634 g) was obtained as orange oil. This was carried forward without further purification.

¹H NMR (400 MHz, DMSO-d₆) δ 12.08 (s, 1H), 4.17 (q, 2H), 4.10 (q, 4H), 2.27 (m, 2H), 2.19 (m, 1H), 2.07 (m, 2H), 2.01 (m, 1H), 1.22 (t, 3H), 1.13 (t, 6H). ¹³C NMR (100 MHz, DMSO-d₆) δ: 172.0, 171.0, 170.8, 170.7, 95.4, 62.0, 61.9, 616.2, 52.9, 28.1, 26.7, 26.4, 14.8, 14.7, 14.5.

### Step c) Synthesis of 4-Oxo-cyclohexanecarboxylic acid (Intermediate C)

4-Hydroxy-cyclohex-3-ene-1,1,3-tricarboxylic acid, triethyl ester (634 g, 2.02 mol) was refluxed 19 hours in a mixture of concentrated hydrochloric acid (600 ml) and water (2900 ml). A 150 ml traction of solvent was distilled out under atmospheric pressure and the residue was filtered through a Celite® bed. The cooled filtrate was saturated with sodium chloride and extracted twice with ethyl acetate (1000 ml). The combined extracts were washed with brine (1000 ml), dried with magnesium sulfate, filtered through a Celite® bed and after solvent was evaporated, the crude product (239 g) was obtained as yellow oil. This was further purified by vacuum distillation, the fraction boiling between 120-245° C./1 mmHg was collected leading to 142 g of colorless liquid, which solidified when it cooled to room temperature. Finally, 132 g of the distilled material was crystallized from 65 ml boiling toluene leading to 4-oxo-cyclohexanecarboxylic acid (63.4 g) as white _{solids.} ¹H NMR (400 MHz, DMSO-d₆) δ: 12.32 (s, 1H), 2.68 (m, 1H), 2.36 (m, 2H), 2.22 (m, 2H), 2.05 (m, 2H), 1.76 (m, 2H), ¹³C NMR (100 MHz, DMSO-d₆) δ 210.5, 176.3, 40.5, 40.0, 28.8.

### Step d) Synthesis of 4-(Carboethoxymelthylene)cyclohexanecarboxylic acid (Intermediate D)

Working under nitrogen pressure, 4-oxo-cyclohexanecarboxylic acid (53.5 g, 376 mmol, 1 eq) was dissolved to 535 ml anhydrous ethanol and 21 wt. % sodium ethoxide in ethanol (146 ml, 30.7 g, 452 mmol, 1.2 eq) was added followed by triethyl phosphonoacetate (82 ml, 92.8 g, 414 mmol, 1.1 eq). The reaction mixture was cooled in an ice bath to 4° C. and 21 wt % sodium ethoxide in ethanol (134 ml, 28.2 g, 414 mmol, 1.1 eq) was added at such a rate the temperature remained between 4-5° C. After the addition, the ice bath was removed, and the reaction was stirred 1 h. The reaction pH was adjusted to pH-5 with glacial acetic acid (50 ml, 52.9 g, 866 mmol, 2.3 eq), solvents were removed by evaporation and the remaining oil was partitioned between isopropyl ether (900 ml) and 1 M hydrochloric acid (900 ml). The organic phase was separated, washed with water (900 ml), brine (900 ml), dried with magnesium sulfate and simultaneously treated 30 min with 5.40 g of activated carbon (Darco® KBB, BNL Fine Chemicals and Reagents). Solids were removed by filtration through a Celite® bed and after solvent evaporation, the crude product (80.6 g) was obtained as yellowish solids. These were crystallized from 355 ml boiling heptanes returning 4-(carbethoxymethylene)cyclohexanecarboxylic acid (62.6 g) as white solids. ¹H NMR (400 MHz, DMSO-d₆) δ 12.17 (s, 1H), 5.62 (s, 1H), 4.02 (q, 2H), 3.43 (m, 1H), 2.47 (m, 1H), 2.25 (m, 1H), 2.16 (m, 2H), 1.93 (m, 2H), 1.46 (m, 2H), 1.15 (t, 3H). ¹³C NMR (100 MHz. DMSO-d₆) δ 176.5, 166.3, 162.2, 114.0, 59.6, 41.9, 35.8, 30.6, 29.9, 28.0, 14.8.

### Step e) Synthesis of 4-(Carbethoxymethyl)cyclohexanecarboxylic acid (Intermediate B)

4-(Carbethoxymethylene)cyclohexanecarboxylic acid (34.6 g, 163 mmol) was dissolved to anhydrous ethanol (350 ml), Palladium 110 wt. % on activated carbon (Aldrich #20,569-9) (3.50 g) was added and heated in an oil bath. When reaction temperature reached 30° C., ammonium formate (25.6 g) was added and heated to 50° C. After 45 minutes, the reaction was allowed to cool down and the catalyst was removed by filtering through a Celite® bed. Solvent was removed by evaporation and the oily residue was partitioned between isopropyl ether (350 ml) and 1 M hydrochloric acid (350 ml). The organic phase was separated, washed with water (350 ml) and brine (350 ml), dried with magnesium sulfate, filtered through a Celite® bed and after solvent evaporation crude 4-(Carbethoxymethyl)cyclohexanecarboxylic acid (33.6 g) was obtained as an oil. A GC-analysis indicated that this material was a 28:72 mixture of cis- and trans-isomers.

### Step f) Synthesis of trans-4-(Carbethoxymethyl)cyclohexanecarboxylic acid (Intermediate F)

A 28:72 mixture of cis and trans-isomers of 4-(carbethoxymethyl)cyclohexanecarboxylic acid (33.6 g) was heated to reflux in 151 ml of hexanes, the heating mantel was removed and stirred 6 hours. The formed solids were collected by filtration and dried 16 hours in a dry6er (55° C.) under reduced pressure returning trans-4-(carbethoxymethyl)cyclohexanecarboxylic acid (17.6 g) as white solids. ¹H NMR (400 MHz, CDCl₃) δ 11.60 (brs, 1H), 4.11 (q, 2H), 2.24(m, 1H), 2.17(d, J=7.05 Hz, 2H), 2.00 (dd, 2H), 1.83(dd, 2H), 1.76 (m, 1H), 1.44 (m, 2H), 1.24 (t, 3H), 1.02 (m, 2H). ¹³C NMR (100 MHz, CDCl₃) δ 182.2, 173.0, 60.5, 42.9, 42.0, 34.3, 32.0, 28.6, 14.5.

In an alternative route to Intermediate F, Intermediate C was prepared by reading ethyl 4-oxocyclohexanecarboxylate (1 equiv), ethanol (10 volumes) and KOH solution (2 equivs. dissolved in 1 volume water) while maintaining temperature below 30° C. Upon reaction completion (about 15 minutes), concentrated HCl (1 volume) was charged with cooling to keep pot temperature below 20° C. The solvent was evaporated and the remainder was diluted with ethyl acetate (10 volumes), 1 N HCl (10 volumes), and brine (10 volumes), stirred, allowed to settle, and the organic layer separated. The aqueous layer was washed layer with ethyl acetate (10 volumes) and the combined organic layers were washed with brine (10 volumes). The resulting material was dried over sodium sulfate and the solids were filtered off. The organic layers, which include Intermediate C, were concentrated to low volume and displace into ethanol (5 volumes) for next step. (80% yield).

4-oxocyclohexanecarboxylic acid, from previous step, (1 equiv) in 5 volumes ethanol, ethanol (5 volumes), 21% NaOEt in ethanol (1.2 equivs) were mixed while maintaining temperature below 25° C. and then stirred about 15 minutes while cooling to 15° C. Triethyl phosphonoacetate (1.1 equiv.) was charged and the reaction cooled to 5° C. 21% NaOEt in ethanol (1.1 equivs.) was charged while maintaining temperature below 10° C. The reaction was warmed to 20° C. and stirred for 30-45 minutes. Upon reaction completion, the reaction was quenched with HOAc (2.3 equivs.) while maintaining temperature below 25° C. The mixture was concentrated to low volume to remove ethanol and diluted with isopropyl ether (15 volumes), 1 N HCl (15 volumes). The mixture was stirred, allowed to settle, and the organic layer was separated. The organic layer was washed with brine (15 volumes) and treated with Darco and sodium sulfate simultaneously. The solids were filtered off. The organic layers, which include Intermediate D, were concentrated to low volume and displace into ethanol (5 volumes) (80% yield).

4-((ethoxycarbonyl)methylene cyclohexanecarboxylic acid, from previous step, (1 equiv) in ethanol (5 volumes), ethanol (5 volumes) and 10% Pd/C (10% by wt) were mixed and heated to 30° C. To the mixture, ammonium formate (2.5 equivs.) was added while continuing to heat to 50° C. The mixture was stirred at 50° C. for 45 minutes, cooled to 20°-30° C. and filtered over Celite. The resultant material was concentrated to low volume to remove ethanol, and diluted with isopropylether (10 volumes) and 1 N HCl (10 volumes). The mixture was stirred, allowed to settle, and the organic layer was separated. The organic layer was washed with water (5 volumes) and brine (10 volumes) and dried over sodium sulfate. The solids were filtered off. The organic layers were concentrated to low volume and displaced into hexanes (5 volumes). The resulting material was heated to reflux to achieve solution and cooled to 15° C. slowly, then granulated for 1 hour at 10° C.-15° C. intermediate F was filtered and dried at 20° C. under reduced pressure. (Overall process yield-25%).

### Preparation 5

### Synthesis of Trans-(4-Chlorocarbonyl-cyclohexyl)-acetic acid ethyl ester

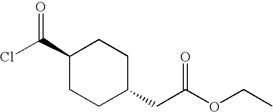

Trans-4-ethoxycarbonylmethyl-cyclohexanecarboxylic acid (Intermediate F) (0.82 g) vas dissolved in THF and stirred at room temperature as thionyl chloride (0.43 mL) was added. After 3 hours, the reaction mixture was concentrated under reduced pressure to afford the title compound ¹H-NMR (CDCl₃) δ: 4.12 (q, 2H) 2.65 (tt, 1H), 2.20 (d, 2H), 2.19 (m, 2H), 1.87 (br d, 2H), 1.78 (m, 1H), 1.53 (br q, 2H), 1.25 (t, 3H), 1.04 (br q,2H).

### Example 24

### (2R,4S)-(3,5-bis-trifluoromethyl-benzyl)-(2-ethyl-6-trifluoromethoxy-1,2,3,4-tetrahydro-quinolin-4-yl)-(2-methyl-2H-tetrazol-5-yl)-amine

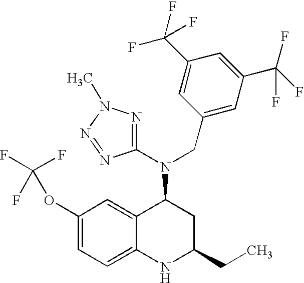

(2R,4S)-1-{4-[(3,5-Bis-trifluoromethyl-benzyl)-(2-methyl-2H-tetrazol-5-yl)-amino]-2-ethyl-6-trifluoromethoxy -3,4-dihydro-2H-quinolin-1-yl}-2,2,2-trifluoro-ethanone (13.3 g) was dissolved in anhydrous tetrahydrofuran (30 ml) and stirred at room temperature as lithium hydroxide monohydrate (3.8 g), 10 ml of water and 10 ml of methanol were added. After the reaction was judged to be complete by thin layer chromatography, the volatiles were removed under reduced pressure and the resulting mixture combined with ethyl acetate and water. The organic layer was separated, dried over sodium sulfate, filtered and concentrated under reduced pressure to afford the crude product, which was purified by silica gel chromatography eluting with 10% ethyl acetate in hexanes to afford the title compound (7.94 g).

MS: 569 [M+H]⁺ found. ¹H-NMR (CDCl₃) δ: 7.72 (bs, 1H), 7.68 (s, 2H), 6.87 (br d, 1H), 6.71 (s, 1H), 6.50 (br d, 1H), 5.80 (br m, 1H), 4.60 (br d, 1H), 4.38 (br d, 1H), 4.17 (s, 3H), 1.37 (m, 1H), 2.516 (br s, 1H), 0.94 (t, 3H).

### Example 25

### Trans-2R,4S)-(4-{4-[(3,5-Bis-trifluoromethyl-benzyl)-(2-methyl-2H-tetrazol-5-yl)-amino]-2-ethyl-6-trifluoromethoxy-3,4-dihydro-2H-quinoline-1-carbonyl}-cyclohexyl)-acetic acid ethyl ester

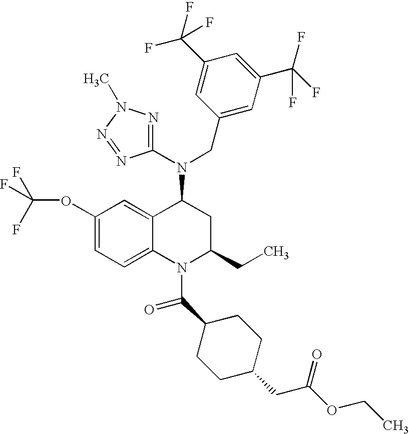

Trans-(4-Chlorocarbonyl-cyclohexyl)-acetic acid ethyl ester obtained from the described procedure was dissolved in 1 mL of dichloromethane and added to a solution of (2R,4S)-(3,5-bis-trifluoromethyl-benzyl)-(2-ethyl-6-trifluoromethoxy-1,2,3,4-tetrahydro-quinolin-4-yl)-(2-methyl-2H-tetrazol-5-yl)-amine (1.0 g) and 0.5 ml of pyridine in 1.0 mL of dichloromethane. After stirring overnight, the reaction mixture was quenched with 2.0 ml of a 2M aqueous sodium hydroxide solution. The mixture was extracted with dichloromethane, the combined organic layers washed sequentially with 1N HCl, saturated aqueous sodium bicarbonate solution, and brine. The organic phase was dried over sodium sulfate, filtered and concentrated under reduced pressure to yield the crude product, which was purified by chromatography on silica gel eluting with 10% ethyl acetate in hexanes to afford 0.8 g of the title compound.

MS: 765 [M+H]⁺ found. ¹H-NMR (CDCl₃) δ: 7.79 (bs, 1H), 7.77 (s, 2H), 7.16 (br s; 2H), 6.79 (s, 1H), 5.10 (br d, 1H), 4.80 (br s, 1H), 4.63 (br s, 1H), 4.16 (s, 3H), 4.10 (q, 2H), 2.53 (br s, 1H), 2.40 (br s, 1H), 2.13 (d, 2H) 1.23 (t, 3H), 0.78 (t, 3H).

### Example 26

### Trans-(2R,4S)-4-{4-[(3,5-Bis-trifluoromethyl-benzyl)-(2-methyl-2H-tetrazol-5-yl)-amino]-2-ethyl-6-trifluoromethoxy-3,4-dihydro-2H-quinoline-1-carbonyl}-cyclohexyl)-acetic acid

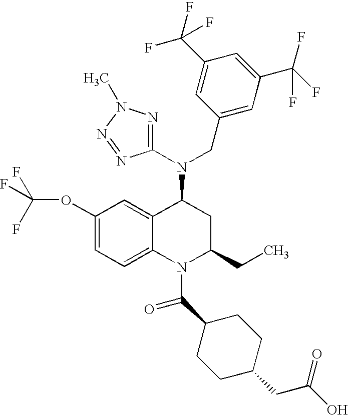

Trans-(2R,4S)-(4-{4-[(3,5-Bis-trifluoromethyl-benzyl)-(2-methyl-2H-tetrazol-5-yl)-amino]-2-ethyl-6-trifluoromethoxy-3,4-dihydro-2H-quinoline-1-carbonyl}-cyclohexyl)-acetic acid ethyl ester (0.70 g) was dissolved in 3 mL of ethyl alcohol and treated with 4N sodium hydroxide (0.15 ml) and heated in a 60° C. oil bath. After 2 hours, the reaction mixture was cooled to room temperature, concentrated under reduced pressure, combined with a 1N aqueous citric acid solution (3.0 ml), and extracted with ethyl acetate. The combined organic layers were dried over sodium sulfate, filtered, and concentrated under reduced pressure to afford 0.60 g of the title compound. MS: 737 [M+H]⁺ found ¹H-NMR (CDCl₃) δ: 7.79 (s, 1H), 7.76 (s, 2H), 7.16 (br s, 2H), 6.79 (s, 1H), 5.10 (br d, 1H), 4.77 (br s, 1H), 4.60 (br s, 1H), 4.16 (s, 3H), 2.53 (m, 1H), 2.41 (m, 1H), 2.18 (d, 2H), 0.78 (t, 3H).

Examples 27-77 were prepared using the analogous methods described above with the appropriate starting acid chlorides.

| | | | Exact/ |
|---|---|---|---|
| | | | Observed |
| Example | IUPAC Name | Structure | Mass (M + 1) |
| 27 | (2R,4S)-(3,5-Bis-trifluoromethyl- benzyl)-(2-ethyl-6- trifluoromethoxy-1,2,3,4- tetrahydro-quinolin-4-yl)-(2- methyl-2H-tetrazol-5-yl)-amine | 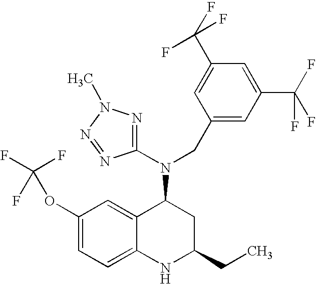 | 568.0/ 569.0 |
| 28 | (2R,4S)-{4-[(3,5-Bis- trifluoromethyl-benzyl)-(2- methyl-2H-tetrazol-5-yl)-amino]- 2-ethyl-6-trifluoromethoxy-3,4- dihydro-2H-quinolin-1-yl}-oxo- acetic acid ethyl ester | 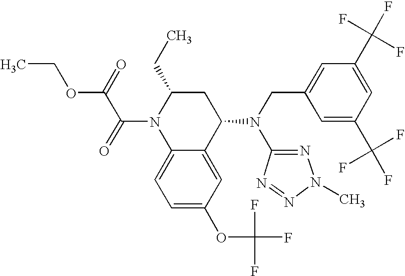 | 668.2/ 669.5 |
| 29 | (2R,4S)-4-{4-[(3,5-Bis- trifluoromethyl-benzyl)-(2- methyl-2H-tetrazol-5-yl)-amino]- 2-ethyl-6-trifluoromethoxy-3,4- dihydro-2H-quinolin-1-yl}-4-oxo- butyric acid methyl ester | 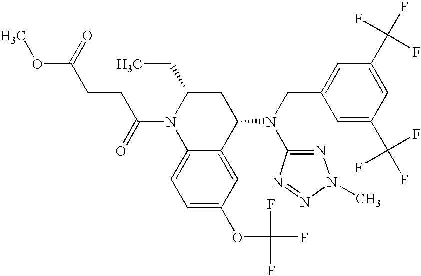 | 682.2/ 683.5 |
| 30 | (2R,4S)-{4-[(3,5-Bis- trifluoromethyl-benzyl)-(2- methyl-2H-tetrazol-5-yl)-amino]- 2-ethyl-6-trifluoromethoxy-3,4- dihydro-2H-quinolin-1-yl}- cyclopentyl-methanone | 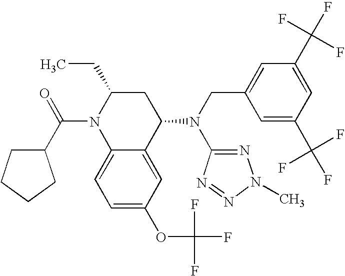 | 664.2/ 665.6 |
| 31 | (2R,4S)-7-{4-[(3,5-Bis- trifluoromethyl-benzyl)-(2- methyl-2H-tetrazol-5-yl)-amino]- 2-ethyl-6-trifluoromethoxy-3,4- dihydro-2H-quinolin-1-yl}-7-oxo- heptanoic acid ethyl ester | 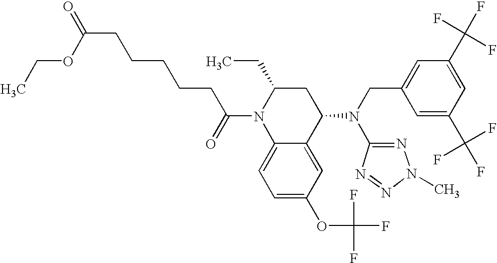 | 738.3/ 739.7 |
| 32 | (2R,4S)-3-{4-[(3,5-Bis- trifluoromethyl-benzyl)-(2- methyl-2H-tetrazol-5-yl)-amino]- 2-ethyl-6-trifluoromethoxy-3,4- dihydro-2H-quinolin-1-yl}-3-oxo- propionic acid ethyl ester | 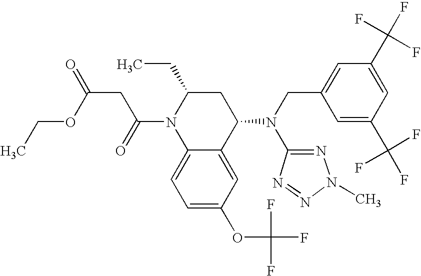 | 682.2/ 683.5 |
| 33 | (2R,4S)-5-{4-[(3,5-Bis- trifluoromethyl-benzyl)-(2- methyl-2H-tetrazol-5-yl)-amino]- 2-ethyl-6-trifluoromethoxy-3,4- dihydro-2H-quinolin-1-yl}-5-oxo- pentanoic acid methyl ester | 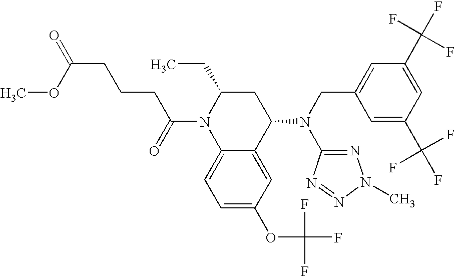 | 696.2/ 697.6 |
| 34 | (2R,4S)-8-{4-[(3,5-Bis- trifluoromethyl-benzyl)-(2- methyl-2H-tetrazol-5-yl)-amino]- 2-ethyl-6-trifluoromethoxy-3,4- dihydro-2H-quinolin-1-yl}-8-oxo- octanoic acid methyl ester | 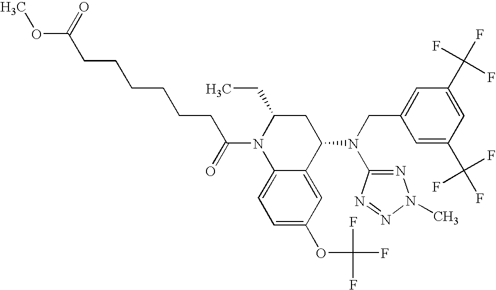 | 738.3/ 739.7 |
| 35 | (2R,4S)-{4-[(3,5-Bis- trifluoromerthyl-benzyl)-(2- methyl-2H-tetrazol-5-yl)-amino]- 2-ethyl-6-trifluoromethoxy-3,4- dihydro-2H-quinolin-1-yl}- cyclohexyl-methanone | 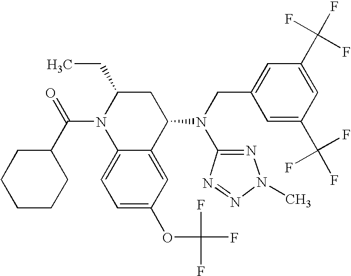 | 696.2/ 697.6 |
| 36 | (2R,4S)-4-{4-[(3,5-Bis- trifluoromethyl-benzyl)-(2- methyl-2H-tetrazol-5-yl)-amino]- 2-ethyl-6-trifluoromethoxy-3,4- dihydro-2H-quinoline-1- carbonyl}-benzoic acid methyl ester | 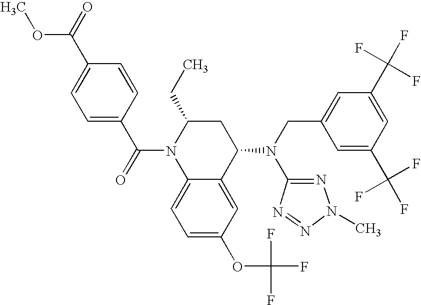 | 730.2/ 731.6 |
| 37 | (2R,4S)-6-{4-[(3,5-Bis- trifluoromethyl-benzyl)-(2- methyl-2H-tetrazol-5-yl)-amino]- 2-ethyl-6-trifluoromethoxy-3,4- dihydro-2H-quinolin-1-yl}-6-oxo- hexanoic acid methyl ester | 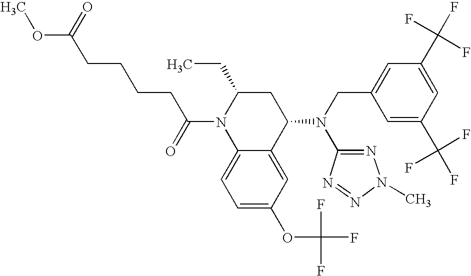 | 710.2/ 711.6 |
| 38 | (2R,4S)-10-{4-[(3,5-Bis- trifluoromethyl-benzyl)-(2- methyl-2H-tetrazol-5-yl)-amino]- 2-ethyl-6-trifluoromethoxy-3,4- dihydro-2H-quinolin-1-yl}-10- oxo-decanoic acid mthyl ester | 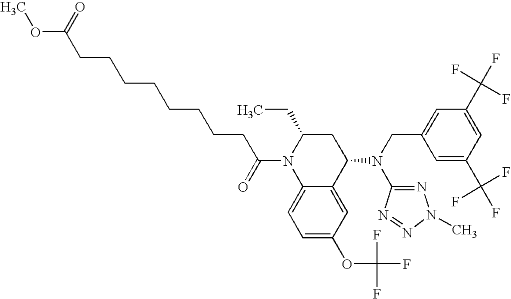 | 766.3/ 767.7 |
| 39 | (2R,4S)-5-{4-[(3,5-Bis- trifluoromethyl-benzyl)-(2- methyl-2H-tetrazol-5-yl)-amino]- 2-ethyl-6-trifluoromethoxy-3,4- dihydro-2H-quinolin-1-yl}- 2,2,3,3,4,4-hexafluoro-5-oxo- pentanoic acid ethyl ester | 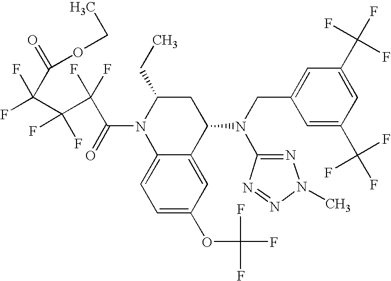 | 818.2/ 819.5 |
| 40 | (2R,4S)-1-{4-[(3,5-Bis- trifluoromethyl-benzyl)-(2- methyl-2H-tetrazol-5-yl)-amino]- 2-ethyl-6-trifluoromethoxy-3,4- dihydro-2H-quinolin-1-yl}-2- cyclopentyl-ethanone | 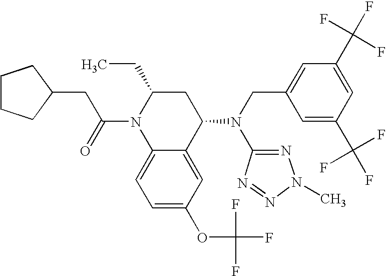 | 678.2/ 679.6 |
| 41 | (2R,4S)-1-{4-[(3,5-Bis- trifluoromethyl-benzyl)-(2- methyl-2H-tetrazol-5-yl)-amino]- 2-ethyl-6-trifluoromethoxy-3,4- dihydro-2H-quinolin-1-yl}-2-ethyl- butan-1-one | 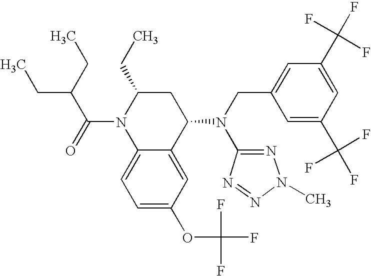 | 666.2/ 667.6 |
| 42 | (2R,4S)-1-{4-[(3,5-Bis- trifluoromethyl-benzyl)-(2- methyl-2H-tetrazol-5-yl)-amino]- 2-ethyl-6-trifluoromethoxy-3,4- dihydro-2H-quinolin-1-yl]-2,2,2- trichloro-ethanone | 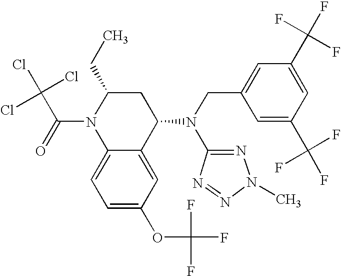 | 714.1/ 714.8 |
| 43 | (2R,4S)-1-{4-[(3,5-Bis- trifluoromethyl-benzyl)-(2- methyl-2H-tetrazol-5-yl)-amino]- 2-ethyl-6-trifluoromethoxy-3,4- dihydro-2H-quinolin-1-yl}-nonan- 1-one | 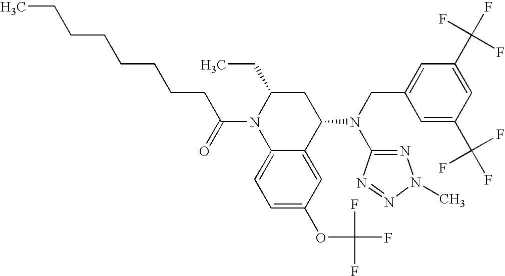 | 708.3/ 709.7 |
| 44 | (2R,4S)-1-{4-[(3,5-Bis- trifluoromethyl-benzyl)-(2- methyl-2H-tetrazol-5-yl)-amino]- 2-ethyl-6-trifluoromethoxy-3,4- dihydro-2H-quinolin-1-yl}-2- phenoxy-ethanone | 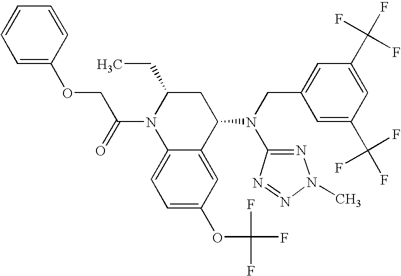 | 702.2/ 703.6 |
| 45 | (2R,4S)-1-{4-[(3,5-Bis- trifluoromethyl-benzyl)-(2- methyl-2H-tetrazol-5-yl)-amino]- 2-ethyl-6-trifluoromethoxy-3,4- dihydro-2H-quinolin-1-yl}-2- methoxy-ethanone | 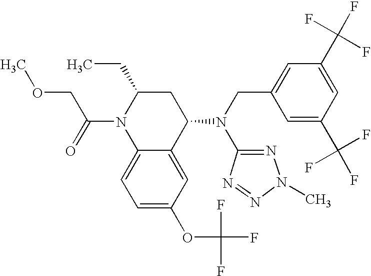 | 640.2/ 641.5 |
| 46 | (2R,4S)-1-{4-[(3,5-Bis- trifluoromethyl-benzyl)-(2- methyl-2H-tetrazol-5-yl)-amino]- 2-ethyl-6-trifluoromethoxy-3,4- dihydro-2H-quinolin-1-yl}-3,3- dimethyl-butan-1-one | 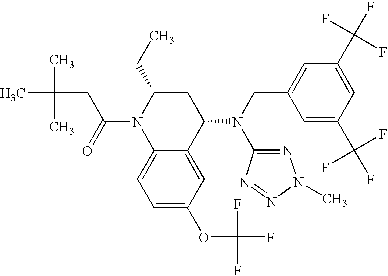 | 666.2/ 667.6 |
| 47 | (2R,4S)-{4-[(3,5-Bis- trifluoromethyl-benzyl)-(2- methyl-2H-tetrazol-5-yl)-amino]- 2-ethyl-6-trifluoromethoxy-3,4- dihydro-2H-quinolin-1-yl}- cyclopropyl-methanone | 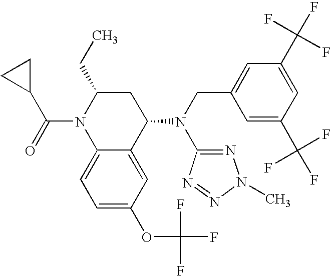 | 636.2/ 637.5 |
| 48 | (2R,4S)-1-{4-[(3,5-Bis- trifluoromethyl-benzyl)-(2- methyl-2H-tetrazol-5-yl)-amino]- 2-ethyl-6-trifluoromethoxy-3,4- dihydro-2H-quinolin-1-yl}- heptan-1-one | 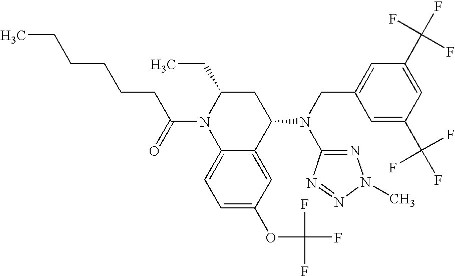 | 680.3/ 681.6 |
| 49 | (2R,4S)-1-{4-[(3,5-Bis- trifluoromethyl-benzyl)-(2- methyl-2H-tetrazol-5-yl)-amino]- 2-ethyl-6-trifluoromethoxy-3,4- dihydro-2H-qui9nolin-1-yl}-2- methyl-propan-1-one | 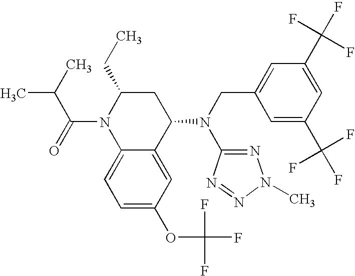 | 638.2/ 639.5 |
| 50 | (2R,4S)-Adamantan-1-yl-{4- [(3,5-bis-trifluoromethyl-benzyl)- (2-methyl-2H-tetrazol-5-yl)- amino]-2-ethyl-6- trifluoromethoxy-3,4-dihydro-2H- quinolin-1-yl}-methanone | 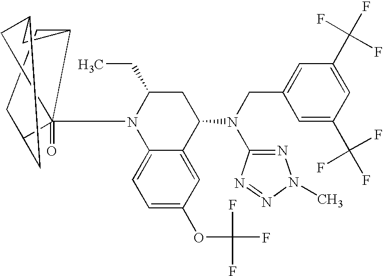 | 730.3/ 731.7 |
| 51 | (2R,4S)-1-{4-[(3,5-Bis- trifluoromethyl-benzyl)-(2- methyl-2H-tetrazol-5-yl)-amino]- 2-ethyl-6-trifluoromethoxy-3,4- dihydro-2H-quinolin-1-yl}-octan- 1-one | 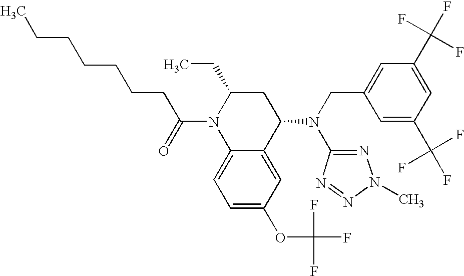 | 694.3/ 695.6 |
| 52 | (2R,4S)-1-{4-[(3,5-Bis- trifluoromethyl-benzyl)-(2- methyl-2H-tetrazol-5-yl)-amino]- 2-ethyl-6-trifluoromethoxy-3,4- dihydro-2H-quinolin-1-yl}-2,2- dimethyl-propan-1-one | 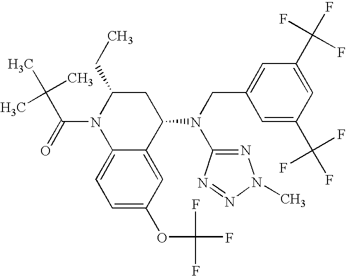 | 652.2/ 653.6 |
| 53 | (2R,4S)-1-{4-[(3,5-Bis- trifluoromthyl-benzyl)-(2- methyl-2H-tetrazol-5-yl)-amino]- 2-ethyl-6-trifluoromethoxy-3,4- dihydro-2H-quinolin-1-yl}-butan- 1-one | 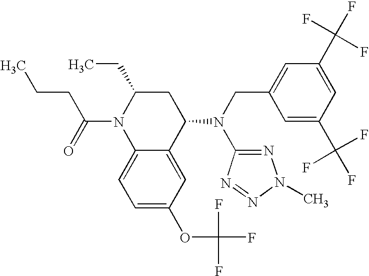 | 638.2/ 639.5 |
| 54 | (2R,4S)-{4-[(3,5-Biss- trifluoromethyl-benzyl)-(2- methyl-2H-tetrazol-5-yl)-amino]- 2-ethyl-6-trifluoromethoxy-3,4- dihydro-2H-quinolin-1-yl}-furan- 2-yl-methanone | 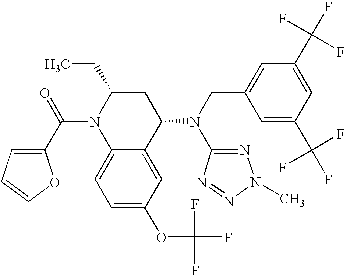 | 662.2/ 663.5 |
| 55 | (2R,4S)-Bicyclo[2.2.1]hept-5-en- 2-yl-{4-[(3,5-bis-trifluoromethyl- benzyl)-(2-methyl-2H-tetrazol-5- yl)-amino]-2-ethyl-6- trifluoromethoxy-3,4-dihydro-2H- quinolin-1-yl}-methanone | 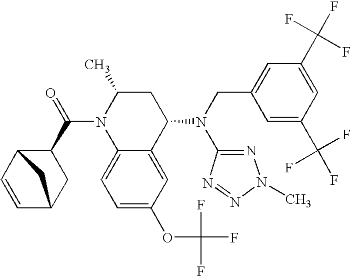 | 688.2/ 689.6 |
| 56 | (2R,4S)-{4-[(3,5-Bis- trifluoromethyl-benzyl)-(2- methyl-2H-tetrazol-5-yl)-amino]- 2-ethyl-6-trifluoromethoxy-3,4- dihydro-2H-quinolin-1-yl}- cyclobutyl-methanone | 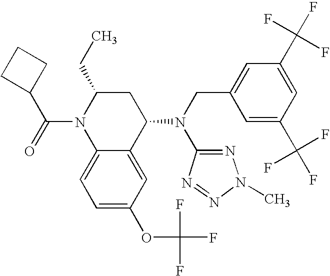 | 650.2/ 651.5 |
| 57 | (2R,4S)-1-{4-[(3,5-Bis- trifluoromethyl-benzyl)-(2- methyl-2H-tetrazol-5-yl)-amino]- 2-ethyl-6-trifluoromethoxy-3,4- dihydro-2H-quinolin-1-yl]-3- methyl-butan-1-one | 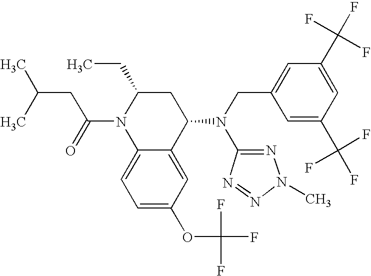 | 652.2/ 653.6 |
| 58 | (2R,4S)-1-{4-[(3,5-Bis- trifluoromethyl-benzyl)-(2- methyl-2H-tetrazol-5-yl)-amino]- 2-ethyl-6-trifluoromethoxy-3,4- dihydro-2H-quinolin-1-yl}-3- cyclopentyl-propan-1-one | 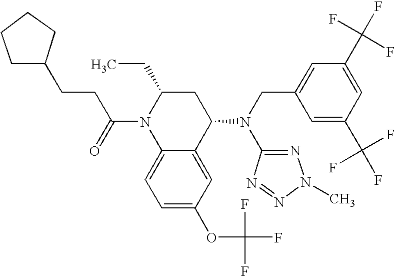 | 692.3/ 693.6 |
| 59 | (2R,4S)-1-{4-[(3,5-Bis- trifluoromethyl-benzyl)-(2- methyl-2H-tetrazol-5-yl)-amino]- 2-ethyl-6-trifluoromethoxy-3,4- dihydro-2H-quinolin-1-yl}- pentan-1-one | 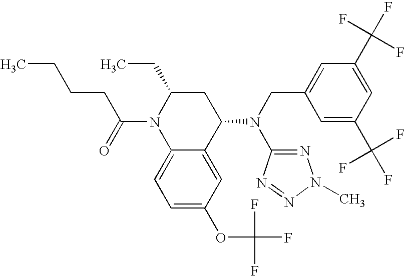 | 692.3/ 693.6 |
| 60 | (2R,4S)-1-{4-[(3,5-Bis- trifluoromethyl-benzyl)-(2- methyl-2H-tetrazol-5-yl)-amino]- 2-ethyl-6-trifluoromethoxy-3,4- dihydro-2H-quinolin-1-yl}-decan- 1-one | 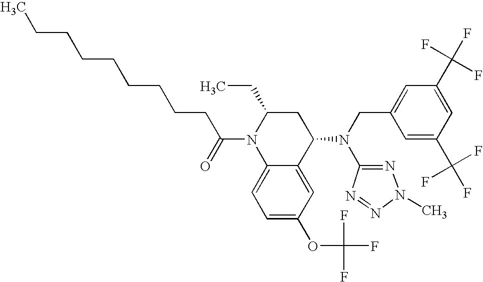 | 722.3/ 723.7 |
| 61 | (2R,4S)-1-{4-[(3,5-Bis- trifluoromethyl-benzyl)-(2- methyl-2H-tetrazol-5-yl)-amino]- 2-ethyl-6-trifluoromethoxy-3,4- dihydro-2H-quinolin-1-yl}-2-ethyl- hexan-1-one | 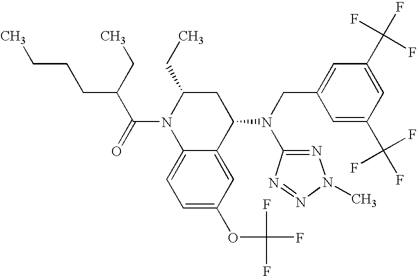 | 694.3/ 695.6 |
| 62 | (2R,4S)-1-{4-[(3,5-Bis- trifluoromethyl-benzyl)-(2- methyl-2H-tetrazol-5-yl)-amino]- 2-ethyl-6-trifluoromethoxy-3,4- dihydro-2H-quinolin-1-yl}- 2,2,3,3,4,4,4-heptafluoro-butan- 1-one | 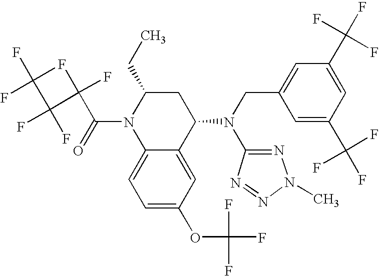 | 764.1/ 765.5 |
| 63 | (2R,4S)-1-{4-[(3,5-Bis- trifluoromethyl-benzyl)-(2- methyl-2H-tetrazol-5-yl)-amino]- 2-ethyl-6-trifluoromethoxy-3,4- dihydro-2H-quinolin-1-yl}-3- methylsulfanyl-propan-1-one | 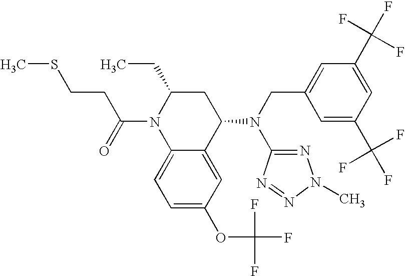 | 670.2/ 671.6 |
| 64 | (2R,4S)-{4-[(3,5-Bis- trifluoromethyl-benzyl)-(2- methyl-2H-tetrazol-5-yl)-amino]- 2-ethyl-6-trifluoromethoxy-3,4- dihydro-2H-quinolin-1-yl}-phenyl- methanone | 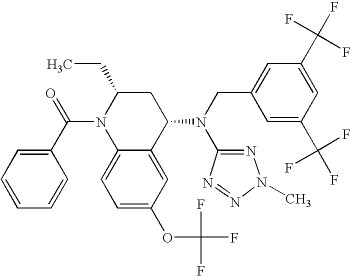 | 672.2/ 673.6 |
| 65 | (2R,4S)-1-{4-[(3,5-Bis- trifluoromethyl-benzyl)-(2- methyl-2H-tetrazol-5-yl)-amino]- 2-ethyl-6-trifluoromethoxy-3,4- dihydro-2H-quinolin-1-yl}-hexan- 1-one | 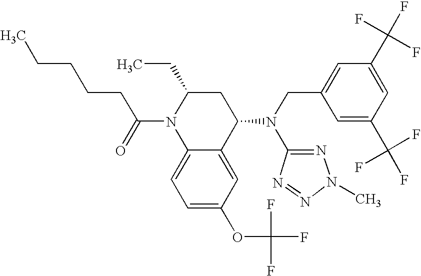 | 670.2/ 671.6 |
| 66 | (2R,4S)-{4-[(3,5-Bis- trifluoromethyl-benzyl)-(2- methyl-2H-tetrazol-5-yl)-amino]- 2-ethyl-6-trifluoromethoxy-3,4- dihydro-2H-quinolin-1-yl}-pyridin- 3-yl-methanone | 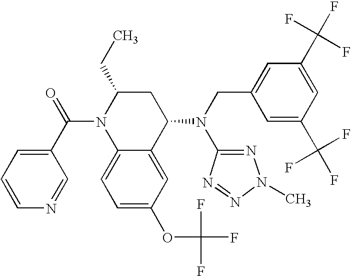 | 673.2/ 674.5 |
| 67 | (2R,4S)-1-{4-[(3,5-Bis- trifluoromethyl-benzyl)-(2- methyl-2H-tetrazol-5-yl)-amino]- 2-ethyl-6-trifluoromethoxy-3,4- dihydro-2H-quinolin-1-yl}-2- dimethylamino-ethanone | 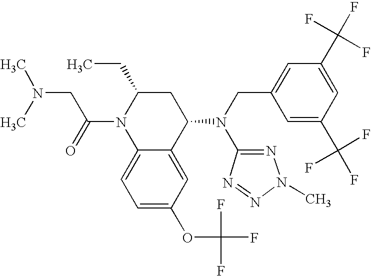 | 653.2/ 654.6 |
| 68 | Trans-(2R,4S)-{4-[(3,5-Bis- trifluoromethyl-benzyl)-(2- methyl-2H-tetrazol-5-yl)-amino]- 2-ethyl-6-trifluoromethoxy-3,4- dihydro-2H-quinolin-1-yl}-(4- propyl-cyclohexyl)-methanone | 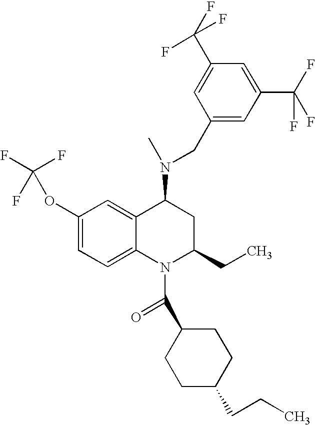 | 720.0/ 721.7 |
| 69 | (2R,4S)-3-{4-[(3,5-Bis- trifluoromethyl-benzyl)-(2- methyl-2H-tetrazol-5-yl)-amino]- 2-ethyl-6-trifluoromethoxy-3,4- dihydro-2H-quinolin-1-yl}-3-oxo- propionic acid | 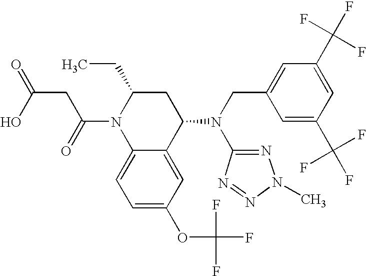 | 654.2/653 (M − 1) |
| 70 | (2R,4S)-4-{4-[(3,5-Bis- trifluoromethyl-benzyl)-(2- methyl-2H-tetrazol-5-yl)-amino]- 2-ethyl-6-trifluoromethoxy-3,4- dihydro-2H-quinolin-1-yl}-4-oxo- butyric acid | 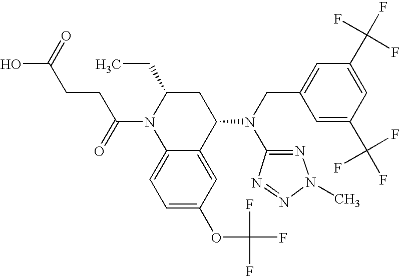 | 668.2/667 (M − 1) |
| 71 | (2R,4S)-5-{4-[(3,5-Bis- trifluoromethyl-benzyl)-(2- methyl-2H-tetrazol-5-yl)-amino]- 2-ethyl-6-trifluoromethoxy-3,4- dihydro-2H-quinolin-1-yl}-5-oxo- pentanoic acid | 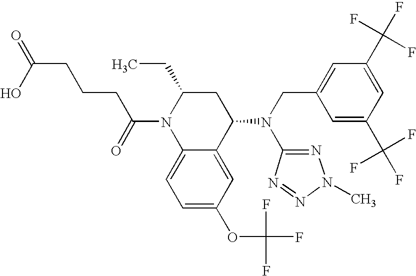 | 682.2/ 681.3 (M − 1) |
| 72 | (2R,4S)-6-{4-[(3,5-Bis- trifluoromethyl-benzyl)-(2- methyl-2H-tetrazol-5-yl)-amino]- 2-ethyl-6-trifluoromethoxy-3,4- dihydro-2H-quinolin-1-yl}-6-oxo- hexanoic acid | 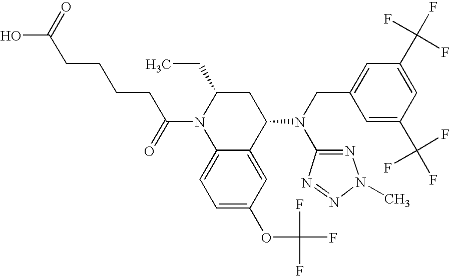 | 696.2/695 (M − 1) |
| 73 | (2R,4S)-7-{4-[(3,5-Bis- trifluoromethyl-benzyl)-(2- methyl-2H-tetrazol-5-yl)-amino]- 2-ethyl-6-trifluoromethoxy-3,4- dihydro-2H-quinolin-1-yl}-7-oxo- heptanoic acid | 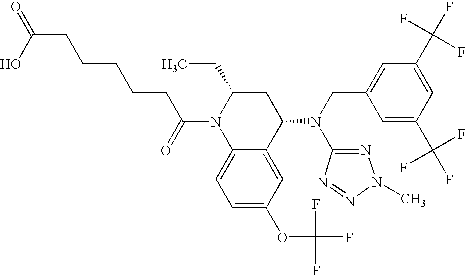 | 710.2/ 709.36 (M − 1) |
| 74 | (2R,4S)-8-{4-[(3,5-Bis- trifluoromethyl-benzyl)-(2- methyl-2H-tetrazol-5-yl)-amino]- 2-ethyl-6-trifluoromethoxy-3,4- dihydro-2H-quinolin-1-yl}-8-oxo- octanoic acid | 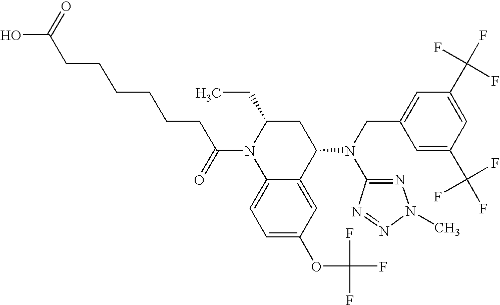 | 724.2/ 723.4 (M − 1) |
| 75 | (2R,4S)-10-{4-[(3,5-Bis- trifluoromethyl-benzyl)-(2- methyl-2H-tetrazol-5-yl)-amino]- 2-ethyl-6-trifluoromethoxy-3,4- dihydro-2H-quinolin-1-yl}-10- oxo-decanoic acid | 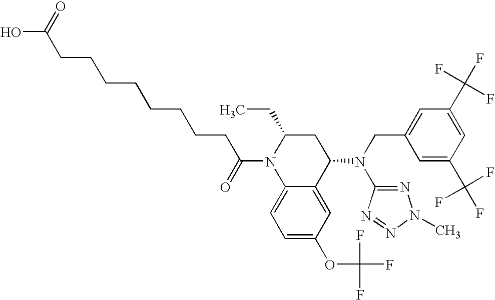 | 752.3/ 751.41 (M − 1) |
| 76 | (2R,4S)-5-{4-[(3,5-Bis- trifluoromethyl-benzyl)-(2- methyl-2H-tetrazol-5-yl)-amino]- 2-ethyl-6-trifluoromethoxy-3,4- dihydro-2H-quinolin-1-yl}- 2,2,3,3,4,4-hexafluoro-5-oxo- pentanoic acid | 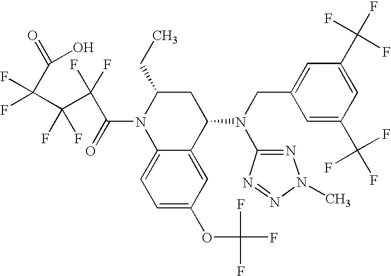 | 790.1/789 (M − 1) |
| 77 | (2R,4S)-5-{4-[(3,5-Bis- trifluoromethyl-benzyl)-(2- methyl-2H-tetrazol-5-yl)-amino]- 2-ethyl-6-trifluoromethoxy-3,4- dihydroi-2H-quinolin-1-yl}-2,2,2- trifluoro-1-oxo-ethane | 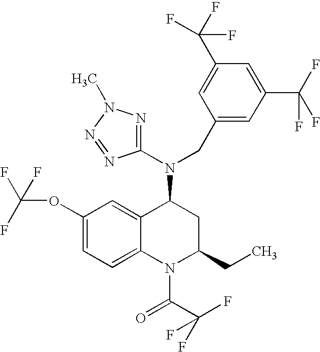 | 664.0/ 665.4 |

### Example 78

### Trans-(2R,4S)-2-(4-{4-[(3,5-Bis-trifluoromethyl-benzyl)-(2-methyl-2H-tetrazol-5-yl)-amino]-2-ethyl-6-trifluoromethoxy-3,4-dihydro-2H-quinoline-1-carbonyl}-cyclohexyl)-acetamide

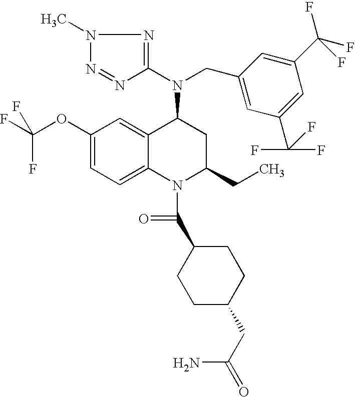

Trans-(2R,4S)-(4-{4-[(3,5-Bis-trifluoromethyl-benzyl)-(2-methyl-2H-tetrazol-5-yl)-amino]-2-ethyl-6-trifluoromethoxy-3,4-dihydro-2H-quinoline-1-carbonyl}-cyclohexyl)-acetic acid ethyl ester (50 mg) in 1.5 mL of anhydrous tetrahydrofuran was treated with thionylchloride (0.5 mL) at room temperature. After 3 hours, the mixture was concentrated under reduced pressure and the residue dissolved in tetrahydrofuran. The resulting solution was cooled in a dry ice/acetone bath as gasous ammonia was condensed into the reaction vessel. After warming to room temperature, the reaction mixture was treated with an aqueous 1N HCl solution and then extracted with ethyl acetate. The combined organic layers were dried over magnesium sulfate, filtered and concentrated under reduced pressure to afford the crude product, which was purified by column chromatography on silica gel eluting with ethyl acetate to afford the title compound (42 mg). MS: 736 [M+H]⁺ found. ¹H-NMR (CDCl₃) δ: 7.79 (s, 1H), 7.76 (s, 2H), 7.16 (br s, 2H), 6.79 (s, 1H), 5.38 (br s, 2H), 5.10 (br d, 1H), 4.76 (m, 1H), 4.60 (m, 1H), 4.16 (s, 3H), 2.53 (m 1H), 2.41 (m, 1H), 2.05 (d, 2H), 0.78 (t, 3H).

Examples 79-87 were prepared using an analogous procedure to those described above using the appropriate starting materials.

### Example 79

### Trans-(2R,4S)-(4-{4-[(3,5-Bis-trifluoromethyl-benzyl)-(2-methyl-2H-tetrazol-5-yl)-amino]-2-methyl-6-trifluoromethyl-3,4-dihydro-2H-quinoline-1-carbonyl}-cyclohexyl)-acetic acid ethyl ester

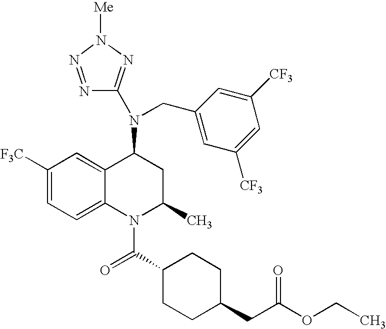

MS: 735 [M+H]⁺ found. ¹H NMR (CDCl₃): δ 0.80 (m, 1H), 0.95 (m, 1H) 1.1 (d, 3H, CH₃), 1.22 (t, 3H, CH₃), 1.4-2.0 (mm, 9H), 2.13 (d, 2H, CH₂), 2.45 (m, 1H, CH), 2.56 (m, 1H, CH), 4.15 (q, 2H, CH₂), 4.18 (s, 3H,NCH₃), 4.6 (bm, 1H, CH), 4.8 (m, 1H, CH), 5.13 (d 1H, CH), 7.1 (s, 1H, CH), 7.26 m, 1H, CH), 7.55 (d, 1H, CH), 7.76 (s, 2H), 7.83 (s, 1H, CH)

### Example 80

### Trans-(2R,2S)-(4-{4-[(3-Chloro-5-trifluoromethyl-benzyl)-(2-methyl-2H-tetrazol-5-yl)-amino]-2-methyl-6-trifluoromethyl-3,4-dihydro-2H-quinoline-1-carbonyl}-cyclohexyl)-acetic acid ethyl ester

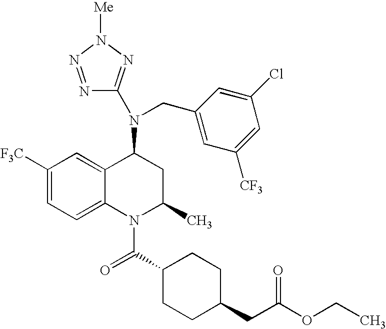

MS: 701 [M+H]⁺ found. ¹HNMR (CDCl₃) δ 0.80 (m, 1H, CH), 0.95 (m, 1H, CH), 1.14 (d, 3H, CH₃), 1.25 (t, 3H, CH₃), 1.25-1.95 (mm, 9H), 2.13 (d, 2H, CH₂), 2.45 (m, 1H, CH), 2.56 (m, 1H CH), 4.16 (q, 2H, CH₂), 4.18 (s, 3H, NCH₃), 4.81 (m, 1H, CH), 5.05 (d, 1H, CH), 7.16 (s, 1H, CH), 7.42-7.57 (m, 4H)

### Example 81

### Trans-(2R,4S)-(4-{4-[(3,5-Dichloro-benzyl)-(2-methyl-2H-tetrazol-5-yl)-amino]-2-methyl-6-trifluoromethyl-3,4-dihydro-2H-quinoline-1-carbonyl}-cyclohexyl)-acetic acid ethyl ester

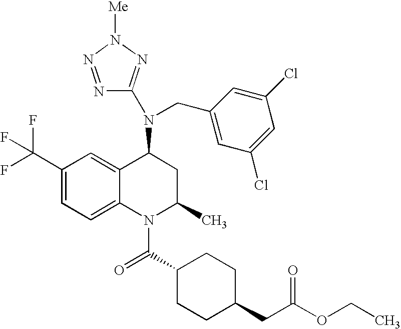

MS: 667 [M+H]⁺ found. ¹HNMR (CDCl₃): δ 0.80 (m, 1H, CH), 0.95 (m, 1H, CH), 1.17 (d, 3H CH₃), 1.22 (t, 3H, CH₃), 1.3-1.93 (mm, 9H) 2.14 (d, 2H, CH₂), 2.47 (m, 1H, CH), 2.57 (m, 1H, CH), 4.15 (q, 2H, CH₂), 4.18 (s, 3H, NCH₃), 4.80 (m, 1H, CH), 5.0 (m, 1H CH), 7.18 (s, 2H), 7.57 (d, 1H, CH)

### Example 82

### Trans-(₂R,4S)-(4-{4-[(3,5-Bis-trifluoromethyl-benzyl)-(2-methyl-2H-tetrazol-5-yl)-amino]-2-methyl-6-trifluorormethyl-3,4-dihydro-2H-quinoline-1-carbonyl}-cyclohexyl)-acetic acid

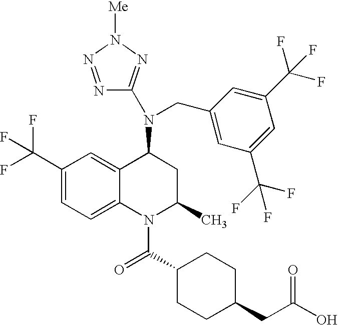

MS: 705 [M−H]− found. ¹HNMR (CDCl₃) δ 0.80 (m, 1H,CH), 1.0 (m, 1H, CH), 1.15 (d, 3H, CH₃), 1.55 (m, 1H, CH), 1.8 (m, 2H, CH₂), 1.95 (m, 2H, CH₂), 2.20 (d, 2H, CH₂), 2.45 (m, 1H, CH), 2.60 (m, 1H, CH), 4.19 (s, 3H, NCH₃), 4.6 (m, 1H, CH), 4.81 (m, 1H, CH), 5.15 (d, 1H, CH), 7.18 (s, 1H, CH), 7.58 (d, 1H, CH), 7.75 (s, 2H), 7.81 (s, 1H, CH)

### Example 83

### Trans-(2R,4S)-(4-{4-[(3,5-Dichloro-benzyl)-(2-methyl-2H-tetrazol-5-yl)-amino]-2-methyl-6-trifluoromethyl-3,4-dihydro-2H-quinoline-1-carbonyl}-cyclohexyl)-acetic acid

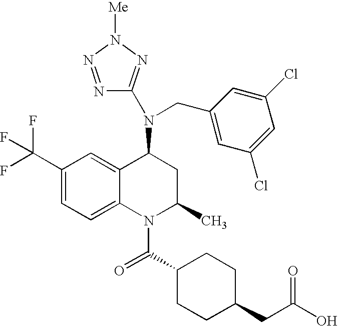

MS: 637 [M−H]− found. ¹HNMR (CDCl₃) δ 0.80 (m, 1H, CH), 0.95 (m, 1H, CH), 1.17 (d, 2H, CH₂), 1.54 (bm, 2H), 1.80 (m, 2H), 1.95 (m, 2H), 2.20 (d, 2H, CH₂), 2.45 (m, 1H, CH), 2.60 (m, 1H, CH), 4.20 (s, 3H, NCH₃), 4.80 (m, 1H, CH), 5.0 (m, 1H, CH), 7.18 (s, 2H), 7.59 (d, 1H, CH)

### Example 84

### Trans-(2R,4S)-(4-{4-[(3-Chloro-5-trifluoromethyl-benzyl)-(2-methyl-2H-tetrazol-5-yl)-amino]-2-methyl-6-trifluoromethyl-3,4-dihydro-2H-quinoline-1-carbonyl}-acetic acid

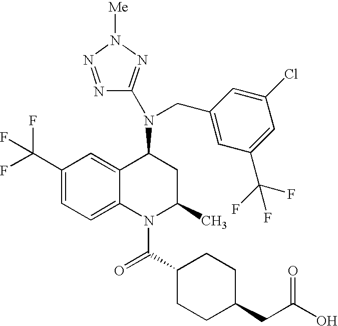

MS: 671 [M−H]− found. ¹HNMR (CD₃OD) δ 0.79 (m, 1H, CH), 1.0 (m, 1H, CH), 1.18 (d, 2H, CH₂), 1.45 (bm, 1H, CH), 1.7 (m 2H), 1.85 (d, 1H, CH), 2.00 (m, 1H, CH), 2.15 (d, 2H, CH₂), 2.5 (m, 1H, CH), 2.65 (m, 1H, CH), 4.19 (s, 3H, NCH₃), 4.75 (m, 2H), 5.05 (d, 1H, CH), 7.10 (s, 1H, CH), 7.42 (d, 1H, CH), 7.5-7.7 (m, 3H)

### Example 85

### Trans-(2R,4S)-2-(4-{4-[(3,5-Bis-trifluoromethyl-benzyl)-(2-methyl-2H-tetrazol-5-yl)-amino]-2-methyl-6-trifluoromethyl-3,4-dihydro-2H-quinoline-1-carbonyl}-cyclohexyl)-acetamide

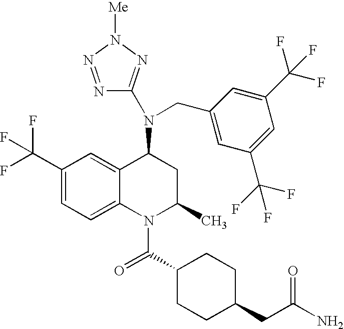

MS: 706 [M+H]⁺ found. ¹HNMR (CDCl³)δ 0.80 (m, 1H, CH), 1.0 (m, 1H, CH), 1.15 (d, 3H, CH₃), 1.51 (bm, 2H), 1.78 (m, 2H, CH₂), 1.96 (m, 3H, CH₂, CH), 2.13 (d, 2H, CH₂), 2.45 (bm, 1H,CH), 2.57 (bm, 1H, CH), 4.17 (s, 3H, NCH₃), 4.62 (bm, 1H, CH), 4.81 (bm, 1H, CH), 5.13 (d, 1H, CH), 6.53 (bs, 2H, CONH₂), 7.15 (s, 1H, CH), 7.24 (m, 1H, CH), 7.55 (d, 1H, CH), 7.75 (s, 2H, CH,CH), 7.78 (s, 1H, CH)

### Example 86

### Trans-(2R,4S)-2-(4-{4-[(3,5-Dichloro-benzyl-(2-methyl-2H-tetrazol-5-yl)-amino]-2-methyl-6-trifluoromethyl-3,4-dihydro-2H-quinoline-1-carbonyl}-cyclohexyl)-acetamide

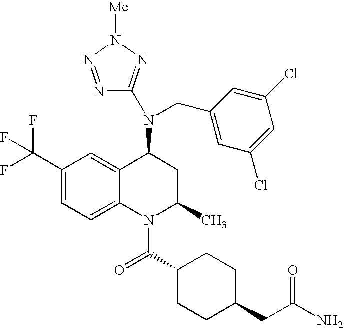

MS: 638 [M+H]⁺ found. ¹HNMR CD₃OD) δ 0.80 (m, 1H, CH), 1.13 (m, 1H, CH), 1.15 (d, 3H, CH₃), 1.4-1.9 (mm, 7H), 2.0 (d,m, 3H, CH₂, CH), 2.50 (m, 1H, CH), 2.65 (m, 1H, CH), 4.18 (s, 3H, NCH₃), 4.67 (d, 1H, CH), 4.79 (m, 1H, CH), 5.0 (d, 1H, CH), 7.10 (s, 1H, CH), 7.34 (d,s, 3H, CH, CH, CH), 7.44 (d, 1H, CH), 7.62 (d, 1H, CH),

### Example 87

### Trans-(2R,4S)-2-(4-{4-(3-Chloro-5-trifluoromethyl-benzyl)-(2-methyl-2H-tetrazol-5-yl)-amino]-2-methyl-6-trifluoromethyl-3,4-dihydro-2H-quinoline-1-carbonyl}-cyclohexyl)-acetamide

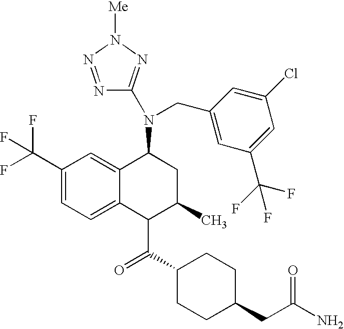

MS: 670 [M−H]− found. ¹HNMR (CD₃OD) δ 0.79 (m, 1H, CH), 1.00 (m, 1H, CH), 1.15 (d, 3H, CH₃), 1.38-1.90 (mm, 7H), 2.10 (d,m, 3H, CH₂, CH), 2.50 (m, 1H, CH), 2.70 (m, 1H, CH), 4.18 (s, 3H, NCH₃), 4.75 (m, 2H, CH, CH), 5.15 (d, 1H, CH), 7.10 (s, 1H, CH), 7.41 (d, 1H, CH), 7.6-7.75 (m, 4H,CH,CH,CH,CH)

### Example 88

### Form A of Trans-(2R,4S)-2-(4-{4-[(3,5-Bis-trifluoromethyl-benzyl)-(2-methyl-2H-tetrazol-5-yl)-amino]-2-ethyl-6-trifluoromethyl-3,4-dihydro-2H-quinoline-1-carbonyl}-cyclohexyl)-acetamide

Trans-(2R,4S)-2-(4-{4-[(3,5-Bis-trifluoromethyl-benzyl)-(2-methyl-2H-tetrazol-5-yl)-amino]-2-ethyl-6-trifluoromethyl-3,4-dihydro-2H-quinoline-1-carbonyl}-cyclohexyl)-acetamide (1.0 gram) was dissolved in 5 ml of ethanol before adding 10 ml of water slowly to afford a cloudy solution. After stirring 4 hours, the resulting suspended solid was collected by vacuum filtration, allowing the sample to dry under a stream of air overnight, to afford the title product as a crystalline solid, Form A (0.6 grams). A sample of Form A was added to silicon oil and observed under cross-polarized light in which it was determined that the sample consisted of material with moderate birefringence and a needle morphology. Using elemental analysis, the following results were obtained: C, 53.30; H, 4.70; N 13.43; (theoretical: C, 53.41; H, 4.73; N, 13.63).

Unless otherwise noted, numerical values described and claimed herein are approximate. Variation within the values may be attributed to equipment calibration, equipment errors, purity of the materials, crystal size, and sample size, among other factors. Additionally, variation may be possible, while still obtaining the same result. For example, X-ray diffraction values are generally accurate to within ±0.2 degrees 2-theta, preferably to within ±0.2 degrees 2-theta. Similarly, DSC results are typically accurate to within about 2° C., preferably to within 1.5° C.

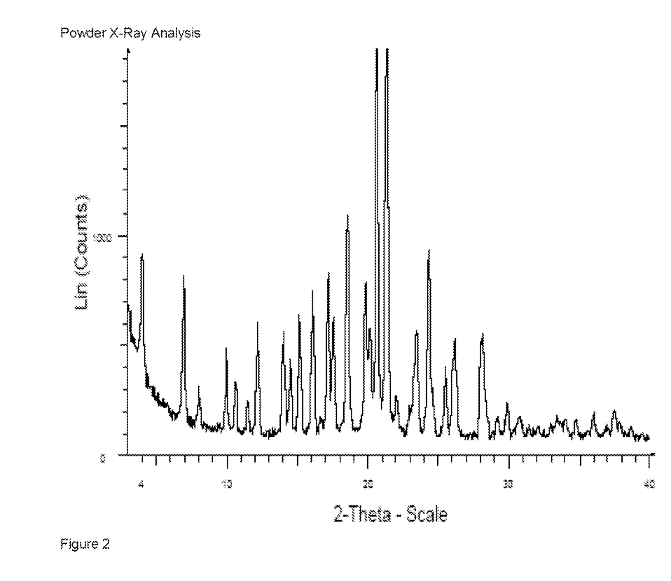

To describe the crystal form, Form A has been examined by powder X-ray diffraction and differential scanning calorimetry (DSC). A discussion of the theory of X-ray power diffraction patterns can be found in Stout & Jensen, X-Ray Structure Determination; A Practical Guide, MacMillan Co. New York, N.Y. (1968), which is incorporated by reference in its entirety for all purposes. Crystallographic data on a collection of powder crystals provides powder X-ray diffraction. Form A has a distinctive powder X-ray diffraction pattern, depicted in FIG. 2 as carried out on a Bruker D5000 diffractometer using copper radiation (wavelength 1.54056A). The tube voltage and amperage were set to 40 kV and 50 mA, respectively. The divergence and scattering slits were set at 1 mm, and the receiving slit was set at 0.6 mm. Diffracted radiation was detected by a Kevex PSI detector. A theta-two theta continuous scan at 2.4°/min (1 sec/0.04° step) from 3.0 to 40° 2θ was used. An alumina standard was analyzed to check the instrument alignment. Data were collected and analyzed using Bruker axis software Version 7.0. Samples were prepared by placing them in a quartz holder. It should be noted that Bruker Instruments purchased Siemans, thus, Bruker D5000 instrument is essentially the same as a Siemans D5000.

In one aspect, the invention is directed to crystalline Form A characterized by the x-ray powder diffraction pattern of FIG. 2 expressed in terms of the degree 2θ, d-spacings, and relative intensities with a relative intensity of ≧5.0% measured on a Bruker D5000 diffractometer with CuKα radiation in Table 1.

TABLE 1

| | | Relative* |
|---|---|---|
| Angle | d | Intensity |
| (Degree 2θ) | (Å) | (≧5.0%) |
| 4.0 | 22.1 | 38.4 |
| 7.0 | 12.7 | 34.3 |
| 8.0 | 11.0 | 12.9 |
| 10.0 | 8.8 | 20.2 |
| 10.6 | 8.3 | 13.9 |
| 11.5 | 7.7 | 10.2 |
| 12.2 | 7.3 | 25.3 |
| 14.0 | 6.3 | 23.3 |
| 14.5 | 6.1 | 18.1 |
| 15.1 | 5.8 | 26.7 |
| 16.1 | 5.5 | 31.3 |
| 16.7 | 5.3 | 7.2 |
| 17.2 | 5.2 | 34.5 |
| 17.6 | 5.0 | 26.4 |
| 18.5 | 4.8 | 45.7 |
| 19.8 | 4.5 | 32.8 |
| 20.2 | 4.4 | 24.0 |
| 20.7 | 4.3 | 84.3 |
| 21.3 | 4.2 | 100.0 |
| 22.0 | 4.0 | 11.3 |
| 23.0 | 3.9 | 9.6 |
| 23.3 | 3.8 | 17.3 |
| 23.5 | 3.8 | 23.8 |
| 24.3 | 3.7 | 38.8 |
| 24.6 | 3.6 | 13.1 |
| 25.5 | 3.5 | 16.7 |
| 26.2 | 3.4 | 22.1 |
| 28.1 | 3.2 | 22.9 |
| 28.4 | 3.1 | 10.3 |
| 29.2 | 3.1 | 7.2 |
| 29.7 | 3.0 | 6.8 |
| 29.9 | 3.0 | 10.0 |
| 30.3 | 2.9 | 5.0 |
| 30.7 | 2.9 | 7.4 |
| 31.4 | 2.8 | 5.6 |
| 31.8 | 2.8 | 5.2 |
| 32.1 | 2.8 | 5.5 |
| 32.5 | 2.8 | 5.0 |
| 33.0 | 2.7 | 5.9 |
| 33.5 | 2.7 | 7.5 |
| 34.1 | 2.6 | 6.9 |
| 34.8 | 2.6 | 6.7 |
| 36.0 | 2.5 | 8.2 |
| 37.0 | 2.4 | 5.7 |
| 37.5 | 2.4 | 8.4 |
| 37.9 | 2.4 | 6.3 |
| 38.7 | 2.3 | 5.2 |

*The relative intensities may change depending on the crystal size and morphology.

The powder X-ray diffraction patterns display high intensity peaks, which are useful in identifying a specific crystal form. However, the relative intensities are dependent upon several factors, including, hut not limited to, crystal size and morphology. As such, the relative intensity values may very from sample to sample. The powder X-ray diffraction values are generally accurate to within ±0.2 degrees 2-theta, due to slight variations of instrument and test conditions. The powder X-ray diffraction pattern or a collective of the diffraction peaks provides a qualitative test for comparison against uncharacterized crystals.

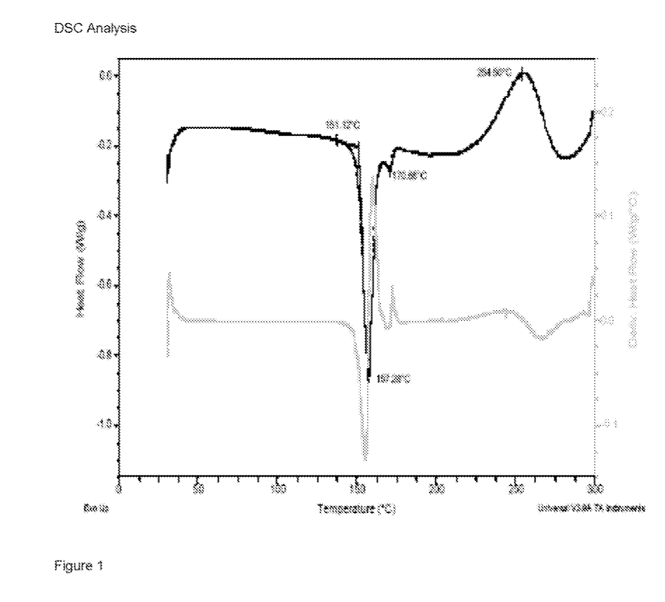

Differential Scanning Calorimetry (DSC) analysis was carried out on either TA Instruments DSC2920 or a Mettler DSC 821, calibrated with indium. DSC sample was prepared by weighing 2-4 mg of material in an aluminum pan with a pinhole. The sample was heated under nitrogen, at a rate of 5° C. per minute from about 30° C. to about 300° C. The onset temperature of the melting endotherm was reported as the melting temperature. The differential scanning calorimetry (DSC) thermogram for Form A is shown in FIG. 1. The onset temperature of the melting endotherm is dependent on the rate of heating, the purity of the sample, crystal size and sample size, among other factors. Typically, the DSC results are accurate to within about ±2° C., preferably to within ±1.5° C. Form A exhibits one major endotherm with an onset temperature of about 151.1° C.

### Example 89

### Solid Amorphous Dispersion Containing Trans-(2R,4S)-2-(4-{4-[(3,5-Bis-trifluoromethyl-benzyl)-(2-methyl-2H-tetrazol-5-yl)-amino]-2-ethyl-6-trifluoromethyl-3,4-dihydro-2H-quinoline-1-carbonyl}-(cyclohexyl)-acetamide “Compound A”

Example 89 contained 25 wt % Trans-(2R,4S)-2-(4-{4-[(3,5-Bis-trifluoromethyl-benzyl)-(2-methyl-2H-tetrazol-5-yl)-amino]-2-ethyl-6-trifluoromethyl-3,4-dihydro-2H-quinoline-1-carbonyl}-cyclohexyl)-acetamide “Compound A” and 75 wt % hydroxypropyl methyl cellulose acetate succinate (HPMCAS; AQOAT “MG” grade, available from Shin Etsu, Tokyo, Japan) in a solid amorphous disperion. Example 89 was prepared by forming a spray solution containing 13.89 g (Compound A, 41.67 g HPMCAS, and 2721 g acetone. The spray solution was primped to a pressure-swirl atomizer (Schlick #2 pressure nozzle) located in a spray-drying chamber. The spray drying chamber consisted of three sections: a top section, a straight-side section, and a cone section. The top section had a diameter of 10.875 inches (27.6 cm), and was equipped with a drying-gas inlet and a spray-solution inlet. The top section also contained an upper perforated plate and a lower perforated plate for dispersing the drying gas within the spray-drying chamber. The upper perforated plate extended across the diameter of the top section and formed an upper chamber in the top section of the spray-drying chamber. The upper perforated plate contained 0.0625-inch (0.16 cm) diameter holes at a uniform spacing of 0.5 inches (1.27-cm). The lower perforated plate contained 0.0625-inch (0.16 cm) diameter holes at a uniform spacing of 0.25 inches (0.64-cm). The drying gas entered the upper chamber in the top section through the drying-gas inlet, at a temperature of about 110° C.

The pressure-swirl atomizer was mounted flush with the bottom of the lower perforated plate. The spray solution was pressurized at a pressure of about 100 psig, with a flow rate of about 26 g/min. The spray solution was then sprayed into the straight-side section of the spray-drying chamber. The straight-side section had a diameter of 10.5 inches (26.7 cm) and a length of 31.75 inches (80.6 cm). The flow rate of drying gas and spray solution were selected such that the atomized spray solution was sufficiently dry by the time it reached the walls of the straight-side section that it did not stick to the walls. The evaporated solvent and drying gas exited the spray drier at a temperature of 45° C.

The solid particles were collected in the cone section of the spray-drying chamber. The cone section had an angle of 58 degrees. The diameter of the cone section at the top was 10.5 inches (26.7 cm), and the distance from the top of the cone section to the bottom was 8.625 inches (21.9 cm). The spray-dried particles, evaporated solvent, and drying gas were removed from the spray-drying chamber through the 1-inch (2.54-cm) diameter outlet port and sent to a cyclone separator where the spray-dried particles were collected. The evaporated solvent and drying gas were then sent to a filter for removal of any remaining particles before discharge.

The solid amorphous dispersion formed using the above procedure was post-dried using a Gruenberg single-pass convection tray drier operating at 40° C. for about 16 hours.

### Concentration Enhancement

In Vitro Microcentrifuge Dissolution Tests

An in vitro dissolution test was used to determine the dissolution performance of the solid amorphous dispersion of Example 89. For this test, a sufficient amount of material was added to a microcentrifuge test tube so that the concentration of Compound A would have been 200 μgA/mL, if all of the compound had dissolved. The test was run in duplicate. The tubes were placed in a 37° C. temperature-controlled chamber, and 1.8 mL PBS at pH 6.5 and 290 mOsm/kg, containing 7.3 mM sodium taurocholic acid and 1.4 mM of 1-palmitoyl-2-oleyl-sn-glycero-3-phosphocholine, was added to each respective tube. The samples were quickly mixed using a vortex mixer for about 60 seconds. The samples were centrifuged at 13,000 G at 37° C. for 1 minute. The resulting supernatant solution was then sampled and diluted 1:5 (by volume) with methanol and analyzed by high-performance liquid chromatography (HPLC). HPLC analysis was performed using a Zorbax RX-C₁₈ column. The mobile phase consisted of 30/70 0.15% trifluoroacetic acid/acetonitrile, with a flow rate of 1.0 mL/min. UV absorbance was measured at 254 nm. The contents of each tube were mixed on the vortex mixer and allowed to stand undisturbed at 37° C. until the next sample was taken. Samples were collected at 4, 10, 20, 40, 90, and 1200 minutes.

A similar test was performed with crystalline Compound A alone, and a sufficient amount of material was added so that the concentration of compound would have been 200 μgA/mL, if all of the compound had dissolved.

The concentrations of Compound A obtained in these samples were used to determine the maximum dissolved concentration of Compound A (“MDC₉₀”) and the area under the concentration-versus-time curve (“AUC₉₀”) during the initial ninety minutes. The results are shown in Table 2.

TABLE 2

| | MDC₉₀ | AUC₉₀ |
|---|---|---|
| Sample | (μgA/mL) | (min * μgA/mL) |
| Example 89 | 148 | 12,800 |
| (25% Compound A: HPMCAS) | | |
| Crystalline Compound A | 13 | 800 |

The dispersion provided an MDC₉₀ that was 11.4-fold that provided by crystalline drug alone, and an AUC₉₀ that was 16.0-fold that provided by crystalline drug alone.

Chemical Stability

The dispersion of Example 89 was stored for 12 weeks at 5° C. closed, 30° C./60% RH open, 40/C25% RH open, or 40° C./75% RH open. “Closed” refers to containers fitted with a threaded cap (limiting exposure to storage conditions). “Open” refers to containers covered loosely with perforated aluminum foil (allowing exposure to storage conditions). Samples were analyzed for Compound A degradation products after 12 weeks, using HPLC to determine the amount of degradant present in the sample. To analyze the samples by HPLC, a sample of the dispersion was dissolved a solvent containing 35/65 0.2% H₃PO₄/acetonitrile. The sample amount was adjusted so that the concentration of active drug in the solution vas about 0.5 mgA/mL. The HPLC method utilized two mobile phases: mobile phase A consisting of 0.2% H₃PO₄, and mobile phase B consisting of acetonitrile. The samples were analyzed using a Waters Symmetry C₈ column, with a solvent flow rate of 1.0 mL/min. Table 3 shows the solvent gradient used.

TABLE 3

| Time | % A | % B |
|---|---|---|
| 0 | 55 | 45 |
| 25 | 35 | 65 |
| 30 | 10 | 90 |
| 35 | 10 | 90 |
| 40 | 55 | 45 |
| 60 | 55 | 45 |

The UV absorbance of Compound A and Compound A impurities were measured at a wavelength of 210 nm. The amide hydrolysis impurity was chosen as the basis for comparison. All impurity peak areas were added and the amide hydrolysis impurity as percent of total peak area was calculated to give the degree of degradation. The results are shown below in Table 4.

TABLE 4

| | Degradant |
|---|---|
| Storage Condition | (%) |
| initial | <LOQ* |
| 5° C., closed | <LOQ |
| 30° C./60% RH | 0.14 |
| 40° C./25% RH | 0.20 |
| 40° C./75% RH | 0.66 |

*<LOQ = less than limit of quantitation

Degradation due to amide hydrolysis was less than 1% after 12 weeks at 40° C./75% RH

In Vivo Tests—Dogs

Samples were dosed orally as suspensions to 3 male beagle dogs in the fasted state. Oral powders for constitution (OPC), were prepared by adding 150 mg of crystalline Compound A to 50 mL water containing 0.5 wt % Methylcellulose A, or 600 mg of the dispersion of Example 1 to 50 mL water containing 0.5 wt % Methylcellulose A and 0.1 wt % Tween 80. Dogs were fasted overnight, and allowed ad libidum access to water. On the morning of the study, approximately 10 mL of OPC solution (3 mgA/kg) was administered via oral gavage with 10 mL normal saline flush.

Whole-blood samples (3-mL red-top Vacutainer tubes without serum separators) were taken from the jugular vein before dosing and at 0.25, 0.5, 1, 2, 4, 6, 8, and 24 hours after dosing. Serum was harvested into cryovials after centrifugation at 3000 rpm for 10 minutes. The samples were frozen and then kept at −20° C. until they were analyzed by liquid chromatography with tandem mass spectrometry (LC/MS/MS). The results are shown in Table 5.

TABLE 5

| | Example 89 | Crystalline |
|---|---|---|
| Parameter | (25% Compound A: HPMCAS) | Compound A |
| Cₘₐₓ (ng/mL) | 1925 | 186 |
| AUC_{0-inf} (ng/mL-hr) | 42,800 | 2693 |

The relative bioavailability (AUC of the test composition divided by AUG of the crystalline drug) for the solid amorphous dispersion of Example 1 was 15.9-fold that of crystalline Compound A alone.

### Example 90

### Solid Amorphous Dispersion of Compound A

Example 90 contained 25 wt % Compound A and 75 wt % hydroxypropyl methyl cellulose (HPMC E3 Prem LV, available from Dow Chemical Co., Midland, Mich.) in a solid amorphous dispersion. Example 90 was prepared by forming a spray solution containing 25.0 mg Compound A, 75.0 mg HPMC, 9.0 g acetone and 1.0 g water. The solution was pumped into a “mini”, spray-drying apparatus via a Cole Parmer 74900 series rate-controlling syringe pump at a rate of 0.65 ml/min. The drug/polymer solution was atomized through a Spraying Systems Co two-fluid nozzle, Model No. SU1A using a heated stream of nitrogen at a flow rate of 0.55 SCFM. The spray solution was sprayed into an 11-cm diameter stainless steel chamber. The heated gas entered the chamber at an inlet temperature of 75° C. and exited at an outlet temperature of 22° C. The resulting solid amorphous dispersion was collected on filter paper, dried under vacuum, and stored in a dessicator. The yield, was about 61%.

### Example 91

### Solid Amorphous Dispersion of Compound A

Example 91 contained 25 wt % Compound A, 60 wt % fumed silica (CAB-O-SIL, available from Cabot Corporation, Tuscola, Ill.), and 15 wt % polyvinyl pyrrolidone (PVP, Plasdone K-15, available from ISP Technologies Inc., Wayne. NJ) in a solid amorphous dispersion, Example 91 was prepared using the mini spray-drier as described above, with the following exceptions. The spray solution contained 25.0 mg Compound A, 60.0 mg CAB-O-SIL, 15.0 mg PVP, and 9.9 g water, the inlet temperature was 70° C., and the yield was about 69%.

### Concentration Enhancement

In Vitro Microcentrifuge Dissolution Tests

An in vitro dissolution test was used to determine the dissolution performance of the formulations of Examples 90 and 91. The tests were performed as described above for Example 89. Results are shown below in Table 6. Crystalline Compound A (from Table 2 is shown again for comparison.

TABLE 6

| | MDC₉₀ | AUC₉₀ |
|---|---|---|
| Sample | (μgA/mL) | (min * μgA/mL) |
| Example 90 | 142 | 12,100 |
| (25% Compound A: HPMC) | | |
| Example 91 | 144 | 12,400 |
| (25% Compound A: CAB-O-SIL PVP) | | |
| Crystalline Compound A | 13 | 800 |

The dispersion of Example 90 provided an MDC₉₀ that was 10.9-fold that provided by crystalline drug alone, and an AUC₉₀ that was 15.1-fold that provided by crystalline drug alone. The drug/substrate adsorbate of Example 91 provided an MDC₉₀ that was 11.1-fold that provided by crystalline drug alone, and an AUC₉₀ that was 15.5-fold that provided by crystalline drug alone.

### Example 92 and 93

### Solid Amorphous Dispersions of Compound A

The solid amorphous dispersions of Examples 92 and 93 were prepared using the mini spray-drier as described above, with the following exceptions. The spray solution for Example 92 contained 23.0 mg Compound A, 23.0 mg HPMCAS (AQOAT “MG” grade, available from Shin Etsu), and 6.1 g acetone, the inlet temperature was 70° C., and the yield was about 62%. The spray solution for Example 93 contained 23.0 mg Compound A, 23.0 mg HPMCAS (AQOAT “HG” grade, available from Shin Etsu) and 6.1 g acetone, the inlet temperature was 70° C., and the yield was about 67%. The grade of HPMCAS used for the dispersion of Example 92 (AQOAT “MG”) contained more acidic groups per mole than the grade of HPMCAS used for the dispersion of Example 93 (AQOAT “HG”).

Chemical Stability

Examples 89 through 93 were stored for 6 weeks at 40° C./75% RH. Samples were analyzed for Compound A degradation products after 6 weeks, using a second HPLC method to determine the amount of degradant present in the sample. To analyze the samples by HPLC, a sample of the dispersion was dissolved a solvent containing 70/30 acetonitrile/water. The sample amount was adjusted so that the concentration of active drug in the solution was about 0.25 mgA/mL. The HPLC method utilized two mobile phases; mobile phase A consisting of 0.1% methanesulfonic acid, and mobile phase B consisting of acetonitrile. The samples were analyzed using an Ace C₈ column, with a solvent flow rate of 0.64 mL/min. Table 7 shows the solvent gradient used.

TABLE 7

| Time | % A | % B |
|---|---|---|
| 0 | 70 | 30 |
| 15 | 15 | 85 |
| 16 | 70 | 30 |
| 20 | 70 | 30 |

The UV absorbance of Compound A and Compound A impurities were measured at a wavelength of 210 nm. A impurity peak areas were added and the amide hydrolysis impurity as percent of total peak area was calculated to give the degree of degradation. The results are shown below in Table 8.

TABLE 8

| | Degradant |
|---|---|
| sample | (%) |
| Example 89 | 0.36 |
| (25% Compound A: HPMCAS) | |
| Example 90 | <LOQ |
| (25% Compound A: HPMC) | |
| Example 91 | <LOQ |
| (25% Compound A: CAB-O-SIL PVP) | |
| Example 92 | 0.22 |
| (50% Compound A: HPMCAS) | |
| Example 93 | 0.17 |
| (50% Compound A: HPMCAS) | |

*<LOQ = less than limit of quantitation

Milliequivalents of acid groups (based on polymer analysis and drug loading in the formulation) increases in the following order: Examples 90 and 91>Example 93>Example 92>Example 89. This corresponds to the amount of degradants observed.

Throughout this application, various publications are referenced. The disclosures of these publications in their entireties are hereby incorporated by reference into this application for all purposes.

It will be apparent to those skilled in the art that various modifications and variations may be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1-22. (canceled)

23. A method for treating atherosclerosis, coronary artery disease, coronary heart disease, coronary vascular disease, peripheral vascular disease, dyslipidemia hyperbetalipoproteinemia, hypoalphalipoproteinemia, hypercholesterolemia, hypertriglyceridemia, familial-hypercholesterolemia or myocardial infarction in a mammal by administering to a mammal in need of such treatment an atherosclerosis, coronary artery disease, coronary heart disease, coronary vascular disease, peripheral vascular disease, dyslipidemia, hyperbetalipoproteinemia, hypoalphalipoproteinemia, hypercholesterolemia, hypertriglyceridemia, familial-hypercholesterolemia or myocardial infarction treating amount of a compound of claim 65 or a pharmaceutically acceptable salt of said compound.

24. A method according to claim 23 wherein atherosclerosis is treated.

25. A method according to claim 23 wherein peripheral vascular disease is treated.

26. A method according to claim 23 wherein dyslipidemia is treated.

27. A method according to claim 23 wherein hyperbetalipoproteinemia is treated.

28. A method according to claim 23 wherein hypoalphalipoproteinemia is treated.

29. A method according to claim 23 wherein familial-hypercholesterolemia is treated.

30. A method according to claim 23 wherein coronary artery disease is treated.

31. A method according to claim 23 wherein myocardial infarction is treated.

32. A pharmaceutical composition which comprises a therapeutically effective amount of a compound of claim 65 or a pharmaceutically acceptable salt of said compound and a pharmaceutically acceptable vehicle, diluent or carrier.

33. A pharmaceutical composition for the treatment of atherosclerosis, coronary artery disease, coronary heart disease, coronary vascular disease, peripheral vascular disease, dyslipidemia, hyperbetalipoproteinemia, hypoalphalipoproteinemia, hypercholesterolemia, hypertriglyceridemia, familial-hypercholesterolemia or myocardial infarction in a mammal which comprises a therapeutically effective amount of a compound of claim 65 or a pharmaceutically acceptable salt of said compound and a pharmaceutically acceptable vehicle, diluent or carrier.

34. A pharmaceutical composition for the treatment of atherosclerosis in a mammal which comprises an atherosclerosis treating amount of a compound of claim 65 or a pharmaceutically acceptable salt of said compound and a pharmaceutically acceptable vehicle, diluent or carrier.

35. A pharmaceutical combination composition comprising: a therapeutically effective amount of a composition comprising
a first compound, said first compound being a compound of claim 65 or a pharmaceutically acceptable salt of said compound;
at least one second compound, said second compound being an HMG CoA reductase inhibitor, an MTP/Apo B secretion inhibitor, a PPAR modulator, an antihypertensive, a bile acid reuptake inhibitor, a cholesterol absorption inhibitor, a cholesterol synthesis inhibitor, a fibrate, niacin, slow-release niacin, a combination of niacin and lovastatin a combination of niacin and simvastatin a combination of niacin and atorvastatin, a combination of amlodipine and atorvastatin, an ion-exchange resin, an antioxidant, an ACAT inhibitor or a bile acid sequestrant, or a pharmaceutically acceptable salt of said second compound; and
a pharmaceutical vehicle, diluent or carrier.

36. A pharmaceutical combination composition according to claim 35 wherein the second compound is an HMG-CoA reductase inhibitor, a PPAR modulator, or niacin.

37. A pharmaceutical combination composition according to claim 36 wherein the second compound is niacin, fenofibrate, lovastatin, simvastatin, pravastatin, fluvastatin, atorvastatin, rivastatin, rosuvastatin or pitavastatin.

38. A pharmaceutical combination composition according to claim 37 further comprising a cholesterol absorption inhibitor.

39. A pharmaceutical combination composition according to claim 35 wherein the cholesterol absorption inhibitor is ezetimibe.

40. A method for treating atherosclerosis in a mammal comprising administering to a mammal in need of treatment thereof;
a first compound, said first compound being a compound of claim 65 a pharmaceutically acceptable salt of said compound; and
at least one second compound, said second compound being an HMG CoA reductase inhibitor, an MTP/Apo B secretion inhibitor, a PPAR modulator, an antihypertensive, a bile acid reuptake inhibitor, a cholesterol absorption inhibitor, a cholesterol synthesis inhibitor, a fibrate, niacin, slow-release niacin, a combination of niacin and lovastatin, a combination of niacin and simvastatin, a combination of niacin and atorvastatin, a combination of amlodipine and atorvastatin, an ion-exchange resin, an antioxidant, an ACAT inhibitor or a bile acid sequestrant, or a pharmaceutically acceptable salt of said second compound;
wherein the amounts of first and second compounds result in a therapeutic effect.

41. A method for treating atherosclerosis according to claim 40 wherein the second compound is an HMG-CoA reductase inhibitor, PPAR modulator, or niacin.

42. A method for treating atherosclerosis according to claim 41 wherein the second compound is niacin, fenofibrate, lovastatin, simvastatin, pravastatin, fluvastatin, atorvastatin, rivastatin, rosuvastatin or pitavastatin.

43. A method for treating atherosclerosis according to claim 42 further comprising administering a cholesterol absorption inhibitor.

44. A method for treating atherosclerosis according to claim 40 wherein the cholesterol absorption inhibitor is ezetimibe.

45. A kit for achieving a therapeutic effect in a mammal comprising packaged in association a first therapeutic agent comprising a therapeutically effective amount of a compound of claim 65 or a pharmaceutically acceptable salt of said compound and a pharmaceutically acceptable carrier, at least one second therapeutic agent, said second therapeutic agent being an HMG CoA reductase inhibitor, an MTP/Apo B secretion inhibitor, a PPAR modulator, an antihypertensive, a bile acid reuptake inhibitor, a cholesterol absorption inhibitor, a cholesterol synthesis inhibitor, a fibrate, niacin, slow-release niacin, a combination of niacin and lovastatin, a combination of niacin and simvastatin, a combination of niacin and atorvastatin, a combination of amlodipine and atorvastatin, an ion-exchange resin, an antioxidant, an ACAT inhibitor or a bile acid sequestrant, or a pharmaceutically acceptable salt of said second therapeutic agent; and a pharmaceutically acceptable carrier and directions for administration of said first and second agents to achieve the therapeutic effect.

46. A kit according to claim 45 wherein said second therapeutic agent comprises an HMG-CoA reductase inhibitor, PPAR modulator, or niacin.

47. A kit according to claim 46 wherein said second therapeutic agent comprises niacin, fenofibrate, lovastatin, simvastatin, pravastatin, fluvastatin, atorvastatin, rivastatin, rosuvastatin or pitavastatin.

48. A kit according to claim 47 further comprising a cholesterol absorption inhibitor.

49. A kit according to claim 45 wherein the cholesterol absorption inhibitor is ezetimibe.

50. A pharmaceutical composition according to claim 34, wherein at least a major portion of the compound of claim 65 is amorphous, and the pharmaceutically acceptable vehicle, diluent or carrier comprises at least one of a polymer and a substrate having a surface area of at least 20 m²/g.

51. A pharmaceutical combination composition according to claim 39, wherein at least a major portion of the compound of claim 65 is amorphous, and the pharmaceutically acceptable vehicle, diluent or carrier comprises at least one of a polymer and a substrate having a surface area of at least 20 m²/g.

52. A pharmaceutical composition according to claim 50, wherein the compound and the polymer are in the form of a solid amorphous dispersion, or the compound is adsorbed onto said substrate.

53. A pharmaceutical combination composition according to claim 51, wherein the compound and the polymer are in the form of a solid amorphous dispersion, or the compound is adsorbed onto said substrate.

54. A pharmaceutical composition according to claim 52, wherein the polymer comprises hydroxypropyl methylcellulose acetate succinate, hydroxypropyl methylcellulose, or polyvinylpyrrolidone.

55. A pharmaceutical composition according to claim 53, wherein the polymer comprises hydroxypropyl methylcellulose acetate succinate, hydroxypropyl methylcellulose, or polyvinylpyrrolidone.

56-64. (canceled)

65. A compound of the Formula I 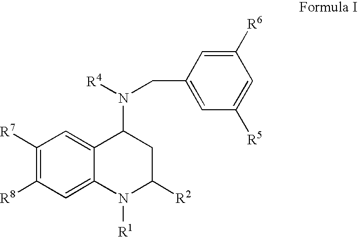
or a pharmaceutically acceptable salt of said compound wherein;
R¹ is Y, W—O—Y or W—Y; wherein W is a carbonyl, Y for each occurrence is independently Z or (C₁-C₁₀)alkyl wherein one of the carbons may be replaced with S, O or N, and when Y is (C₁-C₁₀)alkyl then Y is optionally substituted with one to nine substitutents independently selected from: halo, hydroxy, oxo, amino, amido, carboxy, and Z; wherein Z is a partially saturated, fully saturated or fully unsaturated three to eight membered ring or bicyclic ring system optionally having one to four heteroatoms selected from O, S and N wherein Z is optionally substituted with one, two or three substitutents independently selected from halo, (C₁-C₆)alkyl, hydroxy, (C₁-C₆)alkoxy, amino, amido, cyano, oxo, carboxy, (C₁-C₆)alkyloxycarbonyl, mono-N- and di-N,N—(C₁-C₆)alkylamino wherein said (C₁-C₆)alkyl substituent is optionally substituted with one, two or three substituents independently selected from halo, hydroxy, (C₁-C₆)alkoxy, cyano, oxo, amino, amido, carboxy, mono-N- and di-N,N—C₁-C₆)alkylamino, and (C₁-C₆)alkyloxycarbonyl, said (C₁-C₆)alkyl or (C₁-C₆)alkoxy substituent is also optionally substituted with from one to nine fluorines;
R² is (C₁-C₄)alkyl or (C₁-C₆)cycloalkyl;
R⁴ is V⁰, —COO(C₁-C₄)alkyl, cyano, —CHO, —CONH₂, or —CO(C₁-C₄)alkyl; wherein V⁰ is tetrazolyl, triazolyl, imidazolyl, pyrazolyl, oxadiazolyl, isoxazolyl, furanyl, thiadiazolyl, isothiazolyl, thiophenyl, pyrimidinyl, or pyridinyl; wherein V⁰ is optionally substituted with (R⁰)ₙ wherein n is 1, 2, 3 or 4 and each R⁰ is independently halo, (C₁-C₆)alkyl, hydroxyl, (C₁-C₆)alkoxy, amino, amido, cyano, oxo, carboxamoyl, carboxy, or (C₁-C₆)alkyloxycarbonyl, wherein said (C₁-C₆)alkyl or (C₁-C₆)alkoxy is optionally independently substituted with one or two oxo, one or two hydroxy, or one to nine halo; and
R⁵, R⁶, R⁷, and R⁸ are independently hydrogen, cyano, halo, (C₁-C₄)alkoxy or (C₁-C₄)alkyl wherein said (C₁-C₄)alkyl and (C₁-C₄)alkoxy are optionally substituted independently with from one to seven halo;
with the proviso that when R⁴ is other than V⁰ then R¹ is not (C₁-C₆)alkyl and R¹ has an amido substituent or carboxy substituent.

66. A compound according to claim 65, wherein
R² is methyl, ethyl, 2-propyl, cyclopropyl, ten-butyl, or cyclobutyl;
R⁴ is V⁰ optionally substituted with (R⁰)ₙ; and
R⁵, R⁶, R⁷, and R⁸ are each independently hydrogen, halogen, methyl, cyano, OCF₃ or CF₃.

67. A compound according to claim 66, wherein
R⁴ is tetrazolyl or oxadiazolyl each optionally substituted with (C₁-C₄)alkyl wherein the (C₁-C₄)alkyl is optionally substituted with one to six fluorines.

68. A compound according to claim 67, wherein
R² is ethyl or methyl; and
R⁴ is 2-methyl-tetrazol-5-yl.

69. A compound according to claim 68, wherein
R¹ is W—O—Y; and
Y is methyl, ethyl, 1-propyl, 2-propyl or tert-butyl.

70. A compound according to claim 68, wherein
R¹ is W—Y;
Y is Z or (C₁-C₁₀)alkyl wherein said (C₁-C₁₀)alkyl substituent is optionally substituted with one, two or three substituents independently selected from halo, oxo, amino, amido, (C₁-C₆)alkoxy, carboxy, hydroxy and (C₁-C₆)alkyloxycarbonyl; and
Z is (C₃-C₆)cycloalkyl optionally substituted independently with one or two oxo, amino, amido, carboxy, (C₁-C₆)alkoxy, or (C₁-C₆)alkyl, wherein said (C₁-C₆)alkyl substituent is optionally substituted with one two or three substituents independently selected from halo, oxo, amino, amido, (C₁-C₆)alkoxy, carboxy, hydroxy and (C₁-C₆)alkyloxycarbonyl.

71. A compound according to claim 68, wherein
R¹ is Y;
Y is (C₁-C₆)alkyl substituted with Z; and
Z is (C₃-C₆)cycloalkyl optionally substituted independently with one or two oxo, amino, amido, carboxy, (C₁-C₆)alkoxy, or (C₁-C₅)alkyl, wherein said (C₁-C₆)alkyl substituent is optionally substituted with one, two or three substituents independently selected from halo, oxo, amino, amido, (C₁-C₆)alkoxy, carboxy, hydroxy and (C₁-C₆)alkyloxycarbonyl.

72. A compound according to claim 71, wherein
Z is cyclohexyl optionally substituted with one or two amido, carboxy, (C₁-C₆)alkoxy, or (C₁-C₆)alkyl, wherein said (C₁-C₆)alkyl substituent is optionally substituted with one, two or three substituents selected from halo, oxo, amino, amido, (C₁-C₆)alkoxy, carboxy, hydroxy and (C₁-C₆)alkyloxycarbonyl.

73. A compound according to claim 65, wherein
R² is methyl, ethyl, 2-propyl, cyclopropyl, tert-butyl, or cyclobutyl;
R⁴ is —COO(C₁-C₄)alkyl, cyano, —CHO, —CONH₂, or —CO(C₁-C₄)alkyl; and
R⁵, R⁶, R⁷, and R⁸ are each independently hydrogen, halogen, methyl, cyano, OCF₃ or CF₃.

74. A compound according to claim 73, wherein
R¹ is Y; and
Z is present and Z is (C₃-C₆)cycloalkyl optionally substituted independently with one, two or three halo, hydroxy, amido, carboxy, (C₁-C₆)alkoxy, (C₁-C₆)alkyl, or (C₁-C₆)alkyloxycarbonyl, wherein said (C₁-C₆)alkyl substituent is optionally substituted with one, two or three substituents independently selected from halo, oxo, hydroxyl, amino, amido, (C₁-C₆)alkoxy, carboxy, and (C₁-C₆)alkyloxycarbonyl.

75. A compound according to claim 74 wherein
Y is methyl, ethyl, 1-propyl, 2-propyl or tert-butyl, and Y is substituted with Z; and
Z is cyclobutyl, cyclopentyl, or cyclohexyl, and Z is optionally substituted independently with one or two oxo, amino, amido, carboxy, (C₁-C₆)alkoxy, or (C₁-C₆)alkyl, wherein said (C₁-C₆)alkyl substituent is optionally substituted with one, two or three substituents independently selected from halo, oxo amino, amido, (C₁-C₆)alkoxy, carboxy, hydroxy and (C₁-C₆)alkyloxycarbonyl.

76. A compound according to claim 75, wherein
R² is ethyl or methyl; and
R⁴ is —COOCH₃, cyano, —CHO, —CONH₂, or COCH₃.

77. A compound according to claim 73, wherein
R¹ is W—Y; and
Z is present and Z is (C₃-C₆)cycloalkyl optionally substituted independently with one, two or three halo, hydroxy, amido, carboxy, (C₁-C₆)alkoxy, (C₁-C₆)alkyl, or (C₁-C₆)alkyloxycarbonyl, wherein said (C₁-C₆)alkyl substituent is optionally substituted with one, two or three substituents independently selected from halo, oxo, amino, amido, (C₁-C₆)alkoxy, carboxy, hydroxy and (C₁-C₆)alkyloxycarbonyl.

78. A compound according to claim 77, wherein
R² is ethyl or methyl; and
Z is cyclohexyl optionally substituted with one or two amido, carboxy, (C₁-C₆)alkoxy, or (C₁-C₆)alkyl, wherein said (C₁-C₆)alkyl substituent is optionally substituted with one, two or three substituents selected from halo, oxo, amino, amido, (C₁-C₆)alkoxy, carboxy, hydroxy and (C₁-C₆)alkyloxycarbonyl.

79. A compound according to claim 78, wherein
Z is cyclohexyl substituted with amido, carboxy or (C₁-C₆)alkyl, wherein said (C₁-C₆)alkyl substituent is optionally substituted with halo, oxo, amino, amido, carboxy, hydroxy, or (C₁-C₆)alkyloxycarbonyl.

80. A compound according to claim 65, wherein V⁰ is 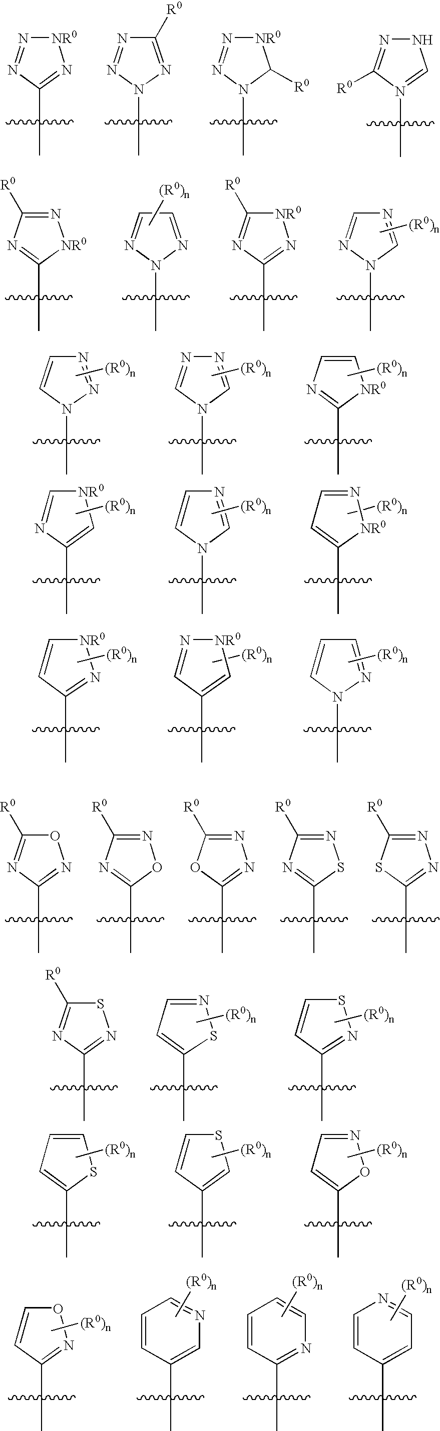 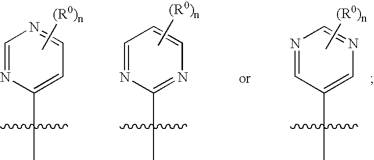
wherein each R⁰ is independently hydrogen, (C₁-C₃)alkoxy, hydroxy, or halo, wherein said (C₁-C₃)alkyl, (C₁-C₃)alkoxy is optionally independently substituted with one to nine halo or one hydroxy.

81. A compound according to claim 80, wherein V⁰ is

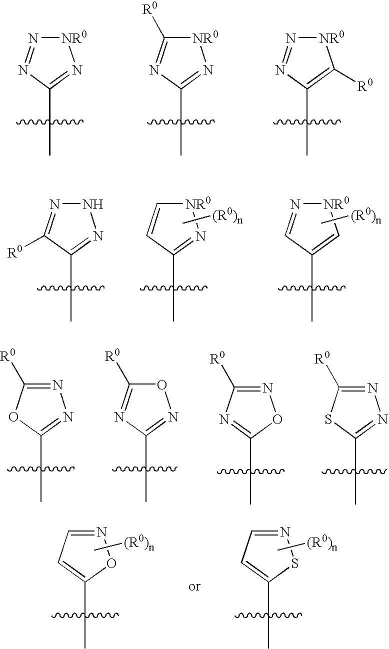

82. A compound according to claim 81, wherein V⁰ is

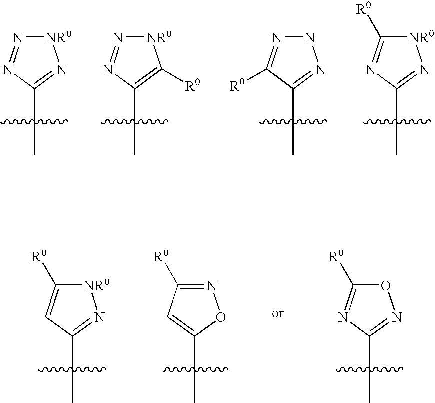

83. A compound according to claim 65, wherein V⁰ is 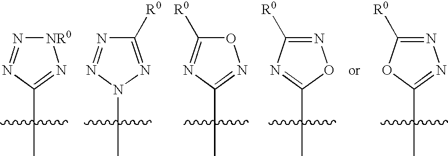
wherein each R⁰ is independently hydrogen, (C₁-C₃)alkyl, (C₁-C₃)alkoxy, hydroxy, or halo, wherein said (C₁-C₃)alkyl or (C₁-C₃)alkoxy is optionally independently substituted with 1 to nine halo or one hydroxy.

84. A compound selected from the group consisting of:
(4-{4-[(3,5-Bis-trifluoromethyl-benzyl)-(2-methyl-2H-tetrazol-5-yl)-amino]-2-ethyl-6-trifluoromethyl-3,4-dihydro-2H-quinoline-1-carbonyl}-cyclohexyl)-acetic acid ethyl ester;
(4-{4-[(3,5-Bis-trifluoromethyl-benzyl)-(2-methyl-2H-tetrazol-5-yl)-amino]-2-ethyl-6-trifluoromethyl-3,4-dihydro-2H-quinoline-1-carbonyl}-cyclohexyl)-acetic acid;
(4-{4-[(3,5-Bis-trifluoromethyl-benzyl)-(2-methyl-2H-tetrazol-5-yl)-amino]-2-methyl-6-trifluoromethyl-3,4-dihydro-2H-quinoline-1-carbonyl}-cyclohexyl)-acetic acid;
[4-(4-(3,5-Bis-trifluoromethyl-benzylcyanamide)-2-ethyl-6-trifluoromethyl-3,4-dihydro-2H-quinoline-1-carbonyl)-cyclohexyl]-acetic acid ethyl ester;
{4-[(3,5-Bis-trifluoromethyl-benzyl)-(2-methyl-2H-tetrazol-5-yl)-amino]-2-ethyl-6-trifluoromethyl-3,4-dihydro-2H-quinolin-1-yl}-[4-(2-hydroxy-ethyl)-cyclohexyl]-methanone:
2-(4-{4-[(3,5-Bis-trifluoromethyl-benzyl)-(2-methyl-2H-tetrazol-5-yl)-amino]-2-ethyl-6-trifluoromethyl-3,4-dihydro-2H-quinolin-1-ylmethyl}-cyclohexyl)-ethanol;
2-(4-{4-[(3,5-Bis-trifluoromethyl-benzyl)-(2-methyl-2H-tetrazol-5-yl)-amino]-2-methyl-6-trifluoromethyl-3,4-dihydro-2H-quinoline-1-carbonyl}-cyclohexyl)-acetamide;
4-[(3,5-Bis-trifluoromethyl-benzyl)-(2-methyl-2H-tetrazol-5-yl)-amino]-2-ethyl-6-trifluoromethyl-3,4-dihydro-2H-quinoline-1-carboxylic acid ethyl ester;
4-{4-[(3,5-Bis-trifluoromethyl-benzyl)-(2-methyl-2H-tetrazol-5-yl)-amino]-2-ethyl-6-trifluoromethyl-3,4-dihydro-2H-quinoline-1-carbonyl}-cyclohexanecarboxylic acid methyl ester;
4-{4-[(3,5-Bis-trifluoromethyl-benzyl)-(2-methyl-2H-tetrazol-5-yl)-amino]-2-ethyl-6-trifluoromethyl-3,4-dihydro-2H-quinoline-1-carbonyl}-cyclohexanecarboxylic acid; and
4-{4-[(3,5-Bis-trifluoromethyl-benzyl)-methoxycarbonyl-amino]-2-ethyl-6-trifluoromethyl-3,4-dihydro-2H-quinoline-1-carbonyl}-cyclohexyl)-acetic acid;
or a pharmaceutically acceptable salt of said compound.

85. A compound selected from the group consisting of:
(2R,4S)-(4-{4-[(3,5-Bis-trifluoromethyl-benzyl)-(2-methyl-2H-tetrazol-5-yl)-amino]-2-methyl-6-trifluoromethyl-3,4-dihydro-2H-quinoline-1-carbonyl}-cyclohexyl)-acetic acid;
(2R,4S)-4-{4-[(3,5-Bis-trifluoromethyl-benzyl)-methoxycarbonyl-amino]-2-ethyl-6-trifluoromethyl-3,4-dihydro-2H-quinoline-1-carbonyl}-cyclohexyl)-acetic acid;
(2R,4S)-(4-{4-[(3,5-Bis-trifluoromethyl-benzyl)-(2-methyl-2H-tetrazol-5-yl)-amino]-2-ethyl-6-trifluoromethyl-3,4-dihydro-2H-quinoline-1-carbonyl}-cyclohexyl)-acetic acid ethyl ester;
(2R,4S)-(4-{4-[(3,5-Bis-trifluoromethyl-benzyl)-(2-methyl-2H-tetrazol-5-yl)-amino]-2-ethyl-6-trifluoromethyl-3,4-dihydro-2H-quinoline-1-carbonyl}-cyclohexyl)-acetic acid;
(2R,4S)-[4-(4-(3,5-Bis-trifluoromethyl-benzylcyanamide)-2-ethyl-6-trifluoromethyl-3,4-dihydro-2H-quinoline-1-carbonyl)-cyclohexyl]-acetic acid ethyl ester;
(2R,4S)-{2-(4-{4-[(3,5-Bis-trifluoromethyl-benzyl)-(2-methyl-2H-tetrazol-5-yl)-amino]-2-methyl-6-trifluoromethyl-3,4-dihydro-2H-quinoline-1-carbonyl}-cyclohexyl)-acetamide;
(2R,4S)-{4-[(3,5-Bis-trifluoromethyl-benzyl)-(2-methyl-2H-tetrazol-5-yl)-amino]-2-ethyl-6-trifluoromethyl-3,4-dihydro-2H-quinolin-1-yl}-[4-(2-hydroxy-ethyl)-cyclohexyl]-methanone;
(2R,4S)-2-(4-{4-[(3,5-Bis-trifluoromethyl-benzyl)-(2-methyl-2H-tetrazol-5-yl)-amino]-2-ethyl-6-trifluoromethyl-3,4-dihydro-2H-quinolin-1-ylmethyl}-cyclohexyl)-ethanol;
(2R,4S)-4-[(3,5-Bis-trifluoromethyl-benzyl)-(2-methyl-2H-tetrazol-5-yl)-amino]-2-ethyl-6-trifluoromethyl-3,4-dihydro-2H-quinoline-1-carboxylic acid ethyl ester;
(2R,4S)-4-{4-(3,5-Bis-trifluoromethyl-benzyl)-(2-methyl-2H-tetrazol-5-yl)-amino]-2-ethyl-6-trifluoromethyl-3,4-dihydro-2H-quinoline-1-carbonyl}-cyclohexanecarboxylic acid methyl ester; and
(2R,4S)-4-{4-[(3,5-Bis-trifluoromethyl-benzyl)-(2-methyl-2H-tetrazol-5-yl)-amino]-2-ethyl-6-trifluoromethyl-3,4-dihydro-2H-quinoline-1-carbonyl}-cyclohexanecarboxylic acid;
or a pharmaceutically acceptable salt of said compound.

86. A compound selected from the group consisting of:
Cis-(2R,4S)-(4-{4-[(3,5-Bis-trifluoromethyl-benzyl)-(2-methyl-2H-tetrazol-5-yl)-amino]-2-methyl-6-trifluoromethyl-3,4-dihydro-2H-quinoline-1-carbonyl}-cyclohexyl)-acetic acid.
Cis-(2R,4S)-2-(4-{4-[(3,5-Bis-trifluoromethyl-benzyl)-(2-methyl-2H-tetrazol-5-yl)-amino]-2-ethyl-6-trifluoromethyl-3,4-dihydro-2H-quinolin-1-ylmethyl}-cyclohexyl)-ethanol;
Cis-(2R,4S)-4-{4-[(3,5-Bis-trifluoromethyl-benzyl)-methoxycarbonyl-amino]-2-ethyl-6-trifluoromethyl-3,4-dihydro-2H-quinoline-1-carbonyl}-cyclohexyl)-acetic acid;
Cis-(2R,4S)-(4-{4-[(3,5-Bis-trifluoromethyl-benzyl)-(2-methyl-2H-tetrazol-5-yl)-amino]-2-ethyl-6-trifluoromethyl-3,4-dihydro-2H-quinoline-1-carbonyl}-cyclohexyl)-acetic acid ethyl ester;
Cis-(2R,4S)-(4-{4-[(3,5-Bis-trifluoromethyl-benzyl)-(2-methyl-2H-tetrazol-5-yl)-amino]-2-ethyl-6-trifluoromethyl-3,4-dihydro-2H-quinoline-1-carbonyl}-cyclohexyl)-acetic acid;
Cis-(2R,4S)-[4-(4-(3,5-Bis-trifluoromethyl-benzylcyanamide)-2-ethyl-6-trifluoromethyl-3,4-dihydro-2H-quinoline-1-carbonyl)-cyclohexyl]-acetic acid ethyl ester;
Cis-(2R,4S)-{4-[(3,5-Bis-trifluoromethyl-benzyl)-(2-methyl-2H-tetrazol-5-yl)-amino]-2-ethyl-6-trifluoromethyl-3,4-dihydro-2H-quinolin-1-yl}-[4-(2-hydroxy-ethyl)-cyclohexyl]-methanone;
Cis-(2R,4S)-2-(4-{4-[(3,5-Bis-trifluoromethyl-benzyl)-2-methyl-2H-tetrazol-5-yl)-amino]-2-methyl-6-trifluoromethyl-3,4-dihydro-2H-quinoline-1-carbonyl}-cyclohexyl)-acetamide;
Cis-(2R,4S)-4-{4-[(3,5-Bis-trifluoromethyl-benzyl)-2-methyl-2H-tetrazol-5-yl)-amino]-2-ethyl-6-trifluoromethyl-3,4-dihydro-2H-quinoline-1-carbonyl}-cyclohexanecarboxylic acid methyl ester;
Cis-(2R,4S)-4-{4-[(3,5-Bis-trifluoromethyl-benzyl)-(2-methyl-2H-tetrazol-5-yl)-amino]-2-ethyl-6-trifluoromethyl-3,4-dihydro-2H-quinoline-1-carbonyl}-cyclohexanecarboxylic acid;
Trans-(2R,4S)-(4-{4-[(3,5-Bis-trifluoromethyl-benzyl)-(2-methyl-2H-tetrazol-5-yl)-amino]-2-methyl-6-trifluoromethyl-3,4-dihydro-2H-quinoline-1-carbonyl}-cyclohexyl)-acetic acid;
Trans-(2R,4S)-4-{4-[(3,5-Bis-trifluoromethyl-benzyl)-methoxycarbonyl-amino]-2-ethyl-6-trifluoromethyl-3,4-dihydro-2H-quinoline-1-carbonyl}-cyclohexyl)-acetic acid;
Trans-(2R,4S)-(4-{4-[(3,5-Bis-trifluoromethyl-benzyl)-(2-methyl-2H-tetrazol-5-yl)-amino]-2-ethyl-6-trifluoromethyl-3,4-dihydro-2H-quinoline-1-carbonyl}-cyclohexyl)-acetic acid ethyl ester;
Trans-(2R,4S)-(4-{4-[(3,5-Bis-trifluoromethyl-benzyl)-(2-methyl-2H-tetrazol-5-yl)-amino]-2-ethyl-6-trifluoromethyl-3,4-dihydro-2H-quinoline-1-carbonyl}-cyclohexyl)-acetic acid;
Trans-(2R,4S)-[4-(4-(3,5-Bis-trifluoromethyl-benzylcyanamide)-2-ethyl-6-trifluoromethyl-3,4-dihydro-2H-quinoline-1-carbonyl)-cyclohexyl]-acetic acid ethyl ester;
Trans-(2R,4S)-{2-(4-{4-[(3,5-Bis-trifluoromethyl-benzyl)-(2-methyl-2H-tetrazol-5-yl)-amino]-2-methyl-6-trifluoromethyl-3,4-dihydro-2H-quinoline-1-carbonyl}-cyclohexyl)-acetamide;
Trans-(2R,4S)-{4-[(3,5-Bis-trifluoromethyl-benzyl)-(2-methyl-2H-tetrazol-5-yl)-amino]-2-ethyl-6-trifluoromethyl-3,4-dihydro-2H-quinolin-1-yl}-[4-(2-hydroxy-ethyl)-cyclohexyl]-methanone;
Trans-(2R,4S)-2-(4-{4-[(3,5-Bis-trifluoromethyl-benzyl)-(2-methyl-2H-tetrazol-5-yl)-amino]-2-ethyl-6-trifluoromethyl-3,4-dihydro-2H-quinolin-1-ylmethyl}-cyclohexyl)-ethanol;
Trans-(2R,4S)-4-{4-[(3,5-Bis-trifluoromethyl-benzyl)-(2-methyl-2H-tetrazol-5-yl)-amino]-2-ethyl-6-trifluoromethyl-3,4-dihydro-2H-quinoline-1-carbonyl}-cyclohexanecarboxylic acid methyl ester; and
Trans-(2R,4S)-4-{4-[(3,5-Bis-trifluoromethyl-benzyl)-(2-methyl-2H-tetrazol-5-yl)-amino]-2-ethyl-6-trifluoromethyl-3,4-dihydro-2H-quinoline-1-carbonyl}-cyclohexanecarboxylic acid;
or a pharmaceutically acceptable salt of said compound.

87. A compound of the Formula II 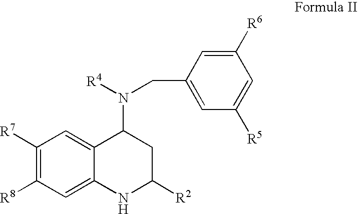
or a pharmaceutically acceptable salt of said compound, wherein
R² is (C₁-C₄)alkyl or (C₁-C₆)cycloalkyl;
R⁴ is tetrazolyl optionally substituted with (R⁰)ₙ wherein n is 1, 2, 3 or 4 and each R⁰ is independently halo, (C₁-C₆)alkyl, hydroxy, (C₁-C₆)alkoxy, amino, amido, cyano, oxo, carboxamoyl, carboxy, or (C₁-C₆)alkyloxycarbonyl, wherein said (C₁-C₆)alkyl or (C₁-C₆)alkoxy is optionally independently substituted with one or two oxo, one or two hydroxy, or one to nine halo; and
R⁵, R⁶, R⁷, and R⁸ are independently hydrogen, cyano, halo, (C₁-C₄)alkoxy or (C₁-C₄)alkyl wherein said (C₁-C₄)alkyl and (C₁-C₄)alkoxy are optionally substituted independently with from one to seven halo.
